(19) United States
(12) Patent Application Publication
(10) Pub. No.: US 2003/0054408 A1
(43) Pub. Date: Mar. 20, 2003

(54) Methods and systems for identifying proteins

(76) Inventors: Ramamoorthi Ravi, Pittsburgh, PA (US); Bjarni Halldorsson, Gaithersburg, MD (US); Alan Koretsky, Bethesda, MD (US); Jonathan Minden, Pittsburgh, PA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/131,266

(22) Filed: Apr. 22, 2002

Related U.S. Application Data
(60) Provisional application No. 60/285,219, filed on Apr. 20, 2001.

(51) Int. Cl. G01N 33/50; C12Q 1/26; G01N 33/53; G06F 19/00; G01N 33/48

(52) U.S. Cl. 435/7.1; 435/25; 702/19

(57) ABSTRACT

A method for identifying a protein including cleaving the protein with a proteolytic agent to produce peptide fragments, providing an array comprising a solution set of binding reagents, contacting the peptide fragments with the array to promote specific interactions between the fragments and the array, detecting the binding pattern of the peptide fragments on the array, and comparing the binding pattern of the peptide fragments to a reference set. A solution set refers to a set of binding reagents, or epitopes associated with such binding reagents, that can identify members of a given protein mixture or protein catalog using a minimal number of binding reagents (or epitopes corresponding to the binding reagents) based on certain constraints. The solution set can be determined using a randomized greedy algorithm. The solution set can be refined using a local search algorithm.

### CLAIM PRIORITY

This application claims priority to U.S. S. No. 60/285,219, entitled “Protein Assignment by Combinatorial Epitope Recognition (PACER)”, and filed on Apr. 20, 2001, naming Jonathan Minden, Ramamoorthi Ravi, Alan Koretsky, and Bjarni Halldorsson as inventors, the contents of which are herein incorporated by reference in their entirety.

### BACKGROUND

1. Field

The methods and systems relate to methods and systems for identifying proteins.

2. Description of Related Art

Proteomics, a major object of post-genome biology, is the analysis of cellular proteins, collectively, the proteome, typically in a high-throughput manner. Elements of proteome analysis include protein separation, protein identification and analysis of protein function. One method for identifying isolated proteins includes mass spectrometry.

One disadvantage of mass spectrometry is the expensive and technologically demanding instrumentation. In mass spectrometry, a protein can be isolated and digested with an amino acid sequence-specific protease. For example, the protease trypsin cleaves proteins at most lysines and arginines (Lill, U., Schreil, A., Henschen, A., Eggerer, H. (1984), “Hysteretic Behavior of Citrate Synthase. Site-Directed Limited Proteolysis,” Eur. J. Biochem. 143, 205-12). The mass of the resulting peptide fragments can be determined by mass spectrometry. The protein can be identified by comparing the spectrum of observed peptide masses to a genomic database of theoretically cleaved proteins. This process is called mass spectrometer fingerprinting and typically employs commercially available software to identify proteins from the profile generated by mass spectrometry. Mass spectrometry is not only expensive and cumbersome, but requires highly trained technicians and has limited sensitivity when a protein is post-transcriptionally modified.

### SUMMARY

The disclosed methods and system include a method for identifying a protein, the method including cleaving the protein with a proteolytic agent to produce peptide fragments, providing an array comprising a solution set of binding reagents, contacting the peptide fragments with the array to promote specific interactions between the fragments and the array, detecting the binding pattern of the peptide fragments on the array; and comparing the binding pattern of the peptide fragments to a reference set.

In an embodiment, disclosed is a method for forming a solution set of at least one epitope, the solution set to identify at least two proteins, the method including forming at least one protein group by associating each of the at least two proteins based on whether the proteins are undistinguished by the solution set, and, updating the solution set with a maximum epitope that divides a maximum number of protein groups.

In an embodiment, disclosed is a method for identifying a solution set of epitopes to identify at least two proteins, the method including determining the epitopes in the at least two proteins based on one or more proteolytic agents, and, applying a randomized greedy algorithm to the determined epitopes to distinguish the solution set of epitopes. The method includes applying a local search algorithm to the solution set, and associating at least one of the epitopes in the solution set with a binding reagent. A binary representation can be generated for the at least two proteins based on the solution set. The randomized greedy algorithm can include forming at least one protein group by associating the at least two proteins based on whether the at least two proteins are distinguished by the solution set. The randomized greedy algorithm can also include identifying a maximum epitope from the determined epitopes where the maximum epitope distinguishes at least as many pairs of the at least two proteins as at least one of the other determined epitopes, associating the maximum epitope with a solution set, removing the maximum epitope from the set of determined epitopes, and, repeating the identifying, associating, and removing until every pair of the at least two proteins are distinguished by the epitopes associated with the solution set.

Also disclosed is a method for identifying at least one protein in a protein catalog, the method including determining epitopes in the protein catalog based on cleaving the protein catalog proteins with at least one proteolytic agent, using a randomized greedy algorithm to identify a solution set of the determined epitopes that can distinguish the protein catalog proteins, forming a chip based on binding reagents associated with the solution set of the determined epitopes, obtaining a signature from the chip based on at least one protein in the protein catalog, and, associating the signature with the at least one protein. In some embodiments, the method further includes identifying a signature for the at least one protein in the protein catalog, the signature based on the solution set of the determined epitopes.

The methods and systems also include a method for generating an identifier for at least one protein in a protein catalog, the method include determining epitopes in the protein catalog based on cleaving the protein catalog proteins with at least one proteolytic agent, identifying a solution set that includes a solution set of determined epitopes that distinguish the proteins, and, associating an identifier with the at least one protein based on whether the at least one protein includes the epitopes in the solution set.

In one embodiment, the methods and systems can provide a processor-readable medium for storing data regarding a protein catalog, the medium including at least one protein name associated with at least one protein catalog protein, and, for each of the at least one protein name, a protein identifier based on a solution set of epitopes for distinguishing the at least one protein catalog protein from other protein catalog proteins, wherein the at least one protein name and the protein identifier are associated. The at least one protein name can be alphanumeric. The protein identifier can be binary and/or alphanumeric. The medium can also include an association with at least one epitope included in the at least one protein catalog protein associated with the at least one protein name. The medium can include an association between the at least one protein name and a protein signature, wherein the protein signature is based upon a chip that includes binding reagents, wherein the binding reagents correspond to the solution set of epitopes. The at least one protein name and the protein identifier can be associated by at least one of at least one database, at least one queue, at least one linked list, at least one hash table, and at least one tree.

In an embodiment, the methods and systems disclose a chip for identifying at least one protein in a protein catalog, where the chip includes binding reagents that are associated with a solution set of epitopes, wherein the solution set of epitopes are determined by a method that includes determining epitopes in the protein catalog based on cleaving the protein catalog proteins with at least one proteolytic agent, initializing the solution set of epitopes to be the empty set, associating protein catalog proteins based on whether the protein catalog proteins are undistinguished by the solution set, updating the solution set with a maximum epitope that divides a maximum number of the associations, and, repeating the associating and updating until the protein catalog proteins are unassociated with any other protein catalog protein.

In some embodiments, disclose is a method for evaluating a set of epitopes for identifying a protein in a protein catalog, the method comprising, providing a chip including binding reagents associated with the set of epitopes, selecting at least two proteins from the protein catalog, determining a signature of the at least two proteins based on the chip, adding errors to the signature to form an augmented signature, and, computing a significance score for unidentified protein in the protein catalog, the significance score based on binding sites in unidentified protein catalog proteins and the augmented signature, identifying a protein from the unidentified protein catalog proteins based on the largest significance score, determining a signature of the identified protein, removing the signature of the identified protein from the augmented signature, repeating computing a significance score for each unidentified protein and identifying, until a number of proteins equal to the at least two selected proteins are identified, and, comparing the identified proteins to the at least two selected proteins.

The methods and systems also include a computer product disposed on a computer readable medium, the computer product for forming a solution set of at least one epitope, the solution set to identify at least two proteins, the computer product including instructions for causing a processor to form at least one protein group by associating each of the at least two proteins based on whether the proteins are undistinguished by the solution set, and, update the solution set with a maximum epitope that divides a maximum number of the protein groups.

Other objects and advantages will become apparent hereinafter in view of the specification and drawings.

### BRIEF DESCRIPTION OF THE DRAWINGS

FIG. 1 is a schematic of a method for identifying a protein.

FIG. 2 provides a method and system that utilize a randomized greedy algorithm to identify a partial solution set of epitopes;

FIG. 3 provides another method and system that utilize a randomized greedy algorithm to identify a partial solution set of epitopes;

FIG. 4 includes an illustrative example of identifying a partial solution set of epitopes;

FIG. 5 provides one illustration for representing proteins in a protein catalog based on a partial solution set of epitopes;

FIG. 6 illustrates a method and system that employs a randomized greedy algorithm with local search;

FIG. 7 is a plot of binding reagent set size versus catalog size.

FIG. 8 provides plots of binding reagent set size versus catalog size for assumptions based on distinguishing protein pairs with minimum size distances of 1, 5, 20, 50, and infinite amino acids.

FIG. 9 is a plot of binding reagent set size versus catalog size for four models of building antibodies.

FIG. 10 includes a method and system for assessing a partial solution set of epitopes for distinguishing proteins; and,

FIG. 11 includes an additional method and system for assessing a partial solution set of epitopes for distinguishing proteins.

### DESCRIPTION

To provide an overall understanding, certain illustrative embodiments will now be described; however, it will be understood by one of ordinary skill in the art that the systems and methods described herein can be adapted and modified to provide systems and methods for other suitable applications and that other additions and modifications can be made without departing from the scope of the systems and methods described herein.

Unless otherwise specified, the illustrated embodiments can be understood as providing exemplary features of varying detail of certain embodiments, and therefore unless otherwise specified, features, components, modules, and/or aspects of the illustrations can be combined, separated, interchanged, and/or rearranged without departing from the disclosed systems or methods.

As used herein, the term “array” refers to a set of binding reagents immobilized onto one or more substrates so that each binding reagent is at a known location. In an exemplary embodiment, a set of binding reagents is immobilized onto a surface in a spatially addressable manner so that each individual binding reagent is located at different and identifiable location on the substrate.

As used herein, the term “binding reagent” refers to a molecule capable of interacting with an epitope as defined herein. Binding reagents having limited cross-reactivity are generally preferred. In certain embodiments, suitable binding reagents include, for example, antibodies, monoclonal antibodies, or derivatives or analogs thereof, including without limitation: Fv fragments, single chain Fv (scFv) fragments, Fab′ fragments, F(ab′)2 fragments, single domain antibodies, camelized antibodies and antibody fragments, and multivalent versions of the foregoing; multivalent binding reagents including without limitation: monospecific or bispecific antibodies, such as disulfide stabilized Fv fragments, scFv tandems ((scFv)₂ fragments), diabodies, tribodies or tetrabodies, which typically are covalently linked or otherwise stabilized (i.e., leucine zipper or helix stabilized) scFv fragments; and other binding reagents including, for example, aptamers and template imprinted materials, such as those of U.S. Pat. No. 6,131,580. In exemplary embodiments, a binding reagent specifically interacts with a single epitope. In other embodiments, a binding reagent may interact with several structurally related epitopes.

As used herein, the term “solution set” refers to an optimum set of binding reagents, or epitopes associated with such binding reagents, as provided by the methods and systems disclosed herein. In some embodiments, for a given protein mixture or protein catalog, multiple solution sets may exist where the multiple solution sets can be of varying size based on varying constraints. Accordingly, the solution sets may include one or more minimum sized solution sets or “minimum sets.” One of ordinary skill in the art can recognize that because the solution sets can be determined using different constraints (e.g., randomly selecting an epitope that performs equally as well as another epitope) for a particular embodiment, a larger size solution set may practically outperform (e.g., better distinguish or identify proteins) a smaller (or minimum size) solution set when considering factors including, for example, protein mixture, false negatives, false positives, and binding reagents that cannot be produced with a desired specificity and/or binding affinity. For example, Table 2, discussed further herein, includes multiple solutions sets of various sizes, including one or more minimum or minimal solution sets, for an exemplary protein catalog. In certain embodiments, proteins in a given protein mixture may be identified based solely on the binding pattern to the solution set. In other embodiments, proteins may be identified based on the binding pattern and optionally other information about the protein, such as, for example, molecular weight or the isoelectric point (pI).

As used herein, the term “reference set” refers to a data set containing information that allows identification of a protein based on its binding pattern to a given set of binding reagents. In exemplary embodiments, a reference set contains information about the binding pattern for members of a protein mixture when contacted with a given solution set of binding reagents. In certain embodiments, the information in the reference set is based on the predicted binding patterns of the proteins in a given protein mixture using predicted digests to determine the epitopes for a protein and correlating these epitopes with the binding reagents in a solution set. Programs for predicting the cleavage pattern of a given protein with a given proteolytic agent may be found, for example, at http://us.expasy.org/tools/peptidecutter/. In other embodiments, the information in the reference set is based on experimental determinations of the binding patterns of the proteins in a given protein mixture to a solution set of binding reagents. In still other embodiments, the information in the reference set is based on a mixture of predicted and experimental data.

As used herein, the term “epitope” refers to a physical structure on a molecule that interacts with a binding reagent. In exemplary embodiments, epitope refers to a desired region on a polypeptide that specifically interacts with a binding reagent. In various specific embodiments, an epitope comprises a label and several adjacent amino acids (e.g., 1, 2, 3, 4, 5, 6, 7, 8, 9 or 10 amino acids adjacent to the label) wherein the label is covalently attached to either the N-terminus or the C-terminus of a polypeptide. In embodiments wherein the label is attached to the N-terminus or the C-terminus of the polypeptide via a linker, the epitope may comprise a label, the linker and several adjacent amino acids (e.g., 1, 2, 3, 4, 5, 6, 7, 8, 9 or 10 amino acids adjacent to the label).

The term “immunogen” traditionally refers to compounds that are used to elicit an immune response in an animal, and is used as such herein. However, many techniques used to produce a desired binding reagent, such as the phage display and aptamer methods described below, do not rely wholly, or even in part, on animal immunizations. Nevertheless, these methods use compounds containing an “epitope,” as defined above, to select for and clonally expand a population of binding reagents specific to the “epitope.” These in vitro methods mimic the selection and clonal expansion of immune cells in vivo, and, therefore, the compounds containing the “epitope” that is used to clonally expand a desired population of phage, aptamers and the like in vitro are embraced within the definition of “immunogens.”

Similarly, the terms “hapten” and “carrier” have specific meaning in relation to the immunization of animals, that is, a “hapten” is a small molecule that contains an epitope, but is incapable as serving as an immunogen, alone. Therefore, to elicit an immune response to the hapten, the hapten is conjugated with a larger carrier, such as bovine serum albumin or keyhole limpet hemocyanin, to produce an immunogen. A preferred immune response would recognize the epitope on the hapten, but not on the carrier. As used herein in connection with the immunization of animals, the terms “hapten” and “carrier” take on their classical definition. However, in the in vitro methods described herein for preparing the desired binding reagents, traditional “haptens” and “carriers” typically have their counterpart in epitope-containing compounds affixed to suitable substrates or surfaces, such as beads and tissue culture plates.

As used herein, the term “protein mixture” refers to a composition of proteins wherein the identity of all, or substantially all, of the proteins in the composition are known. In certain embodiments, the protein mixture may comprise all of the proteins in a given organism, proteome, organ, tissue, cell, organelle, or sub-cellular localization. In other embodiments, the protein mixture may comprise a portion of the proteins from a given organism, proteome, organ, tissue, cell, organelle, or sub-cellular localization, for example, by fractionating the protein mixture based on solubility, charge, hydrophobicity/hydrophilicity, size, isoelectric point (pI), and/or affinity interactions, etc. In other embodiments, the protein mixture may be derived from a transgenic or knockout organism, an organism with a particular disease state, or an organism that has been exposed to a particular environmental condition. In still other embodiments, the protein mixture may comprise two or more purified, or substantially purified, proteins which have been pooled together to form a protein mixture.

As used herein, the terms “label” or “tag” refer to a molecule suitable for detection, such as, for example, spectroscopic detection. In exemplary embodiments, the label is a fluorescent molecule that may be covalently attached to a polypeptide. In exemplary embodiments, suitable fluorescent labels include FAM, JOE, TET, HEX, CY3, CY5, TAMRA, Texas Red™, coumarin, fluorescein, rhodamine, PE, etc. Other suitable fluorescent labels are commercially available from Molecular Probes, Inc. (www.probes.com), such as, for example Alexa™ or BODIPY™. In certain embodiments, the label is directly attached to either the N-terminus or C-terminus of a polypeptide. In other embodiments, the label may be attached to the polypeptide via a linker.

As used herein, the term “proteolytic agent” refers to an agent capable of cleaving a polypeptide into two or more peptide fragments. In certain embodiments, the proteolytic agent may be either a chemical or an enzyme that cleaves a polypeptide in a sequence specific manner. Suitable proteolytic agents include, for example, Arg-C proteinase, Asp-N endopeptidase, BNPS-skatole, caspase 1, caspase 2, caspase 3, caspase 4, caspase 5, caspase 6, caspase 7, caspase 8, caspase 9, caspase 10, chymotrypsin, clostripain (clostridiopeptidase B), CNBr, factor Xa, formic acid, glutamyl endopeptidase, granzyme B, hydroxylamine (NH₂OH), iodosobenzoic acid, lys-C proteinase, NTCB+Ni (2-nitro-5-thiocyanobenzoic acid), pepsin, proline-endopeptidase, proteinase K, staphylococcal peptidase I, thermolysin, thrombin, and trypsin. The cleavage rules for these proteolytic agents are summarized in Table 1.

Table 1. Cleavage rules for exemplary proteolytic agents which may be used in accordance with the methods of the invention. In the table, cleavage occurs between P1 and P1′, P4-P1 are amino acids N-terminal to the cleavage site, and P1′-P2′ are amino acids C-terminal to the cleavage site. Further information about these proteolytic agents may be found at http://us.expasy.org/tools/peptidecutter/.

TABLE 1

| Proteolytic Agent | P4 | P3 | P2 | P1 | P1′ | P2′ |
|---|---|---|---|---|---|---|
| Arg-C proteinase | — | — | — | R | — | — |
| Asp-N endopeptidase | — | — | — | D | — | — |
| BNPS-Skatole | — | — | — | W | — | — |
| Caspase 1 | W or H | E | H | D | — | — |
| Caspase 2 | D | V | A | D | — | — |
| Caspase 3 | D | M | Q | D | — | — |
| Caspase 4 | L | E | V | D | — | — |
| Caspase 5 | L or W | E | H | D | — | — |
| Caspase 6 | V | E | or I | D | — | — |
| Caspase 7 | D | E | V | D | — | — |
| Caspase 8 | I or L | E | T | D | — | — |
| Caspase 9 | L | E | H | D | — | — |
| Caspase 10 | I | E | A | D | — | — |
| Chymotrypsin-high | — | — | — | F or Y | not P | — |
| specificity (C-term to | | | | w | not M | |
| [FYW], not before P) | | | | | or P | |
| Chymotrypsin-low | — | — | — | F,L or Y | not P | — |
| specificity (C-term to | — | — | — | W | not M | — |
| [FYWML], not | | | | | or P | |
| before P) | — | — | — | M | not P or | — |
| | — | — | — | H | not D, M, | — |
| | | | | | P or T | |
| Clostripain | — | — | — | R | — | — |
| (Clostridiopeptidase | | | | | | |
| B) | | | | | | |
| CNBr | — | — | — | M | — | — |
| Factor Xa | A, F, G, I, L, T | D or E | G | R | — | — |
| | V or M | | | | | |
| Formic acid | — | — | — | D | P | — |
| Glutamyl | — | — | — | E | — | — |
| endopeptidase | | | | | | |
| Granzyme B | I | E | P | D | — | — |
| Hydroxylamine | — | — | — | N | G | — |
| Iodosobenzoic acid | — | — | — | W | — | — |
| Lys-C proteinase | — | — | — | not E | K | not DE |
| NTCB (2-nitro-5- | — | — | — | — | C | — |
| thiocyanobenzoic | | | | | | |
| acid) | | | | | | |
| Pepsin (pH1.3) | — | — | — | — | — | — |
| Pepsin (pH>2) | — | — | — | — | — | — |
| Proline-endopeptidase | — | — | H, K or R | P | not P | — |
| Proteinase K | — | — | — | A, E, F, I | — | — |
| | | | | L, T, V, W | | |
| | | | | or Y | | |
| Staphylococcal | — | — | not E | E | — | — |
| peptidase I | | | | | | |
| Thermolysin | — | — | — | not D or E | A, F, I, | |
| | | | | | L, M, or V | |
| Thrombin | — | — | — | R | G | — |
| | A, F, G, I, L, T, V | A, F, G, I, L, | P | R | not D or E | not DE |
| | or M | T, V, W, or A | | | | |
| Trypsin | — | — | — | K or R | not P | — |
| | — | — | W | K | P | — |
| | — | — | NM | R | P | — |

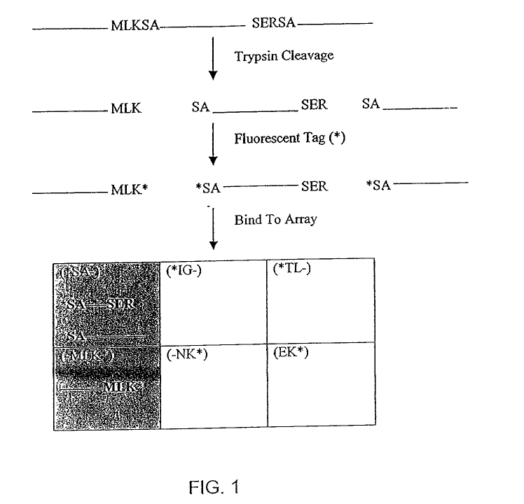

In one embodiment, the invention provides a method for identifying a protein. The method includes the steps of: (a) cleaving the protein with a proteolytic agent, thereby producing two or more peptide fragments; (b) tagging at least one of the N- or C-terminal ends of each of the peptide fragments with a tag; (c) contacting the tagged peptide fragments with an array including a solution set of binding reagents; (d) detecting the binding pattern of the tagged peptide fragments on the array; and (e) comparing the binding pattern of the tagged protein fragments to a reference set. This process is illustrated schematically in FIG. 1, in which a protein is cleaved into peptide fragments and contacted with a solution set comprising six binding reagents.

In another embodiment (PACER I), the invention provides a method for identifying a solution set of binding reagents capable of identifying each protein in a given protein mixture. The choice of epitopes to which binding reagents are targeted may be optimized based on what is known of the protein mixture (e.g., the sequences of the proteins in the mixture) and the peptide fragments that are generated when the proteins are cleaved with a given proteolytic agent. A solution set of binding reagents may be designed to recognize a set of epitopes that are shared by many proteins in such a way that the set of epitopes covers and is capable of distinguishing proteins in the protein mixture. The set of binding reagents will be selected with the objective that each protein will be recognized by a unique set of binding reagents, i.e., each protein will have a unique signature of binding to the binding reagents. The choice of epitopes to which the binding reagents will be made may be optimized according to the set-covering algorithm described below in Example 1.

In various embodiments, a solution set of binding reagents may be a generic set, directed to permutations of 1 to n amino acids adjacent to a tag attached to the N-terminus or C-terminus of a peptide fragment. In this generic set, n may be about 1, 2, 3, 4, 5, 6, 7, 8, 9 or 10 amino acids, typically n is about 2-5 amino acids, more typically n is about 2 amino acids. The tag may be attached to the N-terminal or the C-terminal end of a peptide fragment so as to form an epitope comprising the tag and 1 to n amino acids adjacent to the tag. The generic set may include binding reagents capable of interacting with peptide fragments tagged at the C-terminus, the N-terminus, or a mixture thereof. The number of C-terminal or N-terminal amino acids, n, is selected to produce an optimal number of binding reagents capable of distinguishing most proteins in a protein mixture, and preferably permitting the deconvolution of a mixture of from 2 to about 10 or more proteins, typically from about 2 to about 5 proteins.

In certain embodiments, the binding reagents are directed to generic epitopes (e.g., not directed to ends produced with a specific proteolytic agent) comprising X₁-X₂-Tag and Tag-Y₁-Y₂, where X₁, X₂, Y₁ and Y₂ may be any amino acid. This set optimally includes (20×20)+(20×20)=800 binding reagents. The tag may be attached to the peptide fragments using chemistries appropriate for attachment to the N-terminal (e.g., the tag of Tag-Y₁-Y₂ will be attached via an amine group) and C-terminal (e.g., the tag of X₁-X₂-Tag would be attached via a carboxyl group) peptide ends. If one or more binding reagents cannot be made specific to any given epitope, additional binding reagents may be made. For instance, if Tag-Y₁-Y₂ cannot be made where Y₁ is a specific amino acid, then Tag-Y₁-Y₂-Y₃ may be selected, where Y₁ is the specific amino acid and Y₂ and Y₃ are any amino acids. This may accumulate to nineteen binding reagents to the solution set, but would permit complete set coverage under substantially all circumstances. This completely generic set would permit use of multiple proteases for the same protein sample, thereby increasing the resolution of the assay which may be useful for identifying proteins from a large protein mixture.

In other embodiments, the binding reagents may be designed so as to interact with epitopes generated using a particular sequence specific proteolytic agent. As shown above in Table 1, certain proteolytic agents cleave before or after a particular amino acid residue thereby consistently producing fragments with a fixed amino acid at the N-terminus or C-terminus of the resultant peptide fragments. For example, trypsin cleaves C-terminal to Arg or Lys except when followed by a proline. Thus, after cleavage with trypsin, the resulting peptide fragments will typically contain Arg or Lys at the C-terminus and will not contain Arg, Lys or Pro at the N-terminus. Therefore, if the protein to be identified is digested with trypsin, the binding reagents may be designed to recognize epitopes comprising the structures: X₁-X₂-Lys-Tag and Tag-Y₁-Y₂, where Y₁ is any amino acid except Arg, Lys or Pro, and X₁, X₂ and Y₂ are any amino acid except Arg or Lys. Therefore, the number of discrete binding reagents in this set is (18×18)+(17×18)=630. Optionally, if any combination of X₁ and X₂ or Y₁ and Y₂ is incapable of generating a binding reagent specific to that combination, additional, more complex immunogens might be used to produce binding reagents, typically by adding additional amino acids to the immunogen. Binding reagents sets may designed that are specific for peptides produced with any given proteolytic agents, for example, as shown in Table I above.

In yet another embodiment, the binding reagent solution set may include a mixture of generic (e.g., not tailored to peptides produced with a specific proteolytic enzyme) and specific binding reagents (e.g., tailored to peptides produced with a specific proteolytic agent). For example, the solution set may include binding reagents capable of binding peptides including the structures X₁-X₂-Lys-Tag and Tag-Y₁-Y₂, where X₁ and X₂ are any amino acid except Lys and Arg and Y₁ and Y₂ are any amino acid. This set would include (18×18)+(20×20)=724 binding reagents. In this set, the C-terminal tagged epitopes would be specific to trypsin digests, while the N-terminal tagged epitopes are generic. In this embodiment, the chemistries for addition of the tag to both the C-terminal (Lys) end and the N-terminal end of the peptide fragments would be the same. This set may be desirable if a trypsin digest cannot resolve certain proteins in a given protein mixture because a second proteolytic agent other than trypsin may be used to cleave the protein sample and produce a second binding pattern, specific to the second proteolytic agent, so as to aid in identifying the protein. Additional binding reagents may be prepared, as above, if one or more of the described binding reagents cannot be made. Similar composite binding reagent sets (e.g., comprising binding reagents for specific and generic epitopes) may be constructed for proteolytic agents other trypsin, including, for example, any of the proteolytic agents shown in Table 1.

In the various embodiments, whether the binding reagent set is generic or specifically tailored to a selected proteolytic agent and/or protein mixture, a goal in determining a solution set of binding reagents is not that the binding reagents each recognize distinct epitopes within the protein mixture (e.g., a one-to-one correlation of binding reagents to proteins in a given mixture), but that many or all of the binding reagents recognize epitopes common to peptide fragments produced from cleavage of a plurality of proteins within a given mixture. A concurrent goal is that the minimum set produces a unique binding pattern for the peptide fragments of each protein within the mixture. Thus, in one embodiment, a solution set of binding reagents provides for identification of a maximum number of proteins within a given protein mixture using the fewest number of binding reagents possible.

As discussed above, if any specific binding reagent in the above-described embodiments cannot be practically made, an alternative or supplemental binding reagent or binding reagent set may be constructed. However, since a given solution set may include a relatively large number of potential binding reagents, a cross-reactivity or lack of binding may be accounted for by the algorithm or computational method used to identify proteins based on their pattern of binding to the given solution set. Therefore, in many cases, additional binding reagents need not be made even if a particular binding reagent does not show the desired specificity for a given epitope. Accordingly, the methods of the invention will remain effective for identifying proteins even in the presence of inaccuracies such as lack of a certain binding reagent or cross-reactivity. In the presence of such inaccuracies, it may be useful to cleave aliquots of the protein sample with different proteolytic agents and determine the binding pattern of the peptide fragments to the solution set for the different digests.

In an exemplary embodiment, the protein to be identified may be isolated from the protein mixture prior to cleavage with the proteolytic agent (e.g., the protein sample substantially comprises only a single protein). In other embodiments, the protein to be identified is only partially purified from the protein mixture and may be part of a sample comprising two or more proteins. When using a solution set of binding reagents, it may be possible to identify the proteins in a mixture containing from about 2 to about 10 different proteins, more typically, from about 2 to about 5 different proteins, in a single assay. As the size of the set of binding reagents is increased, it may be possible to identify proteins within protein samples containing even larger numbers of proteins, for example from about 2 to about 20 proteins, from about 2 to about 50 proteins, or from about 2 to about 100 or more different proteins in a single assay.

In various embodiments, the proteins to be analyzed may be purified or partially purified using any method capable of fractionating proteins, including, for example, gel electrophoresis, column chromatography, affinity interactions, etc. In exemplary embodiments, the protein sample is extracted by conventional methods from a two-dimensional polyacrylamide gel electrophoresis (2D PAGE) gel comprising the complete protein mixture. 2D PAGE gels are described in detail in U.S. Pat. Nos. 6,043,025 and 6,127,134 and typically separate proteins in a first dimension by isoelectric focusing and in a second dimension by mass. In other embodiments, proteins may be purified or partially purified using chromatographic methods, such as column chromatography, HPLC, and the like. In certain embodiments, orthogonal information, such as mass and charge information, may be used to assist in the identification of the protein, as discussed below.

The isolated protein or partially purified protein mixture is then cleaved with a proteolytic agent. In exemplary embodiments, the protein sample is digested with a sequence-specific proteolytic agent, such as, for example, Arg-C proteinase, Asp-N endopeptidase, BNPS-skatole, caspase 1, caspase 2, caspase 3, caspase 4, caspase 5, caspase 6, caspase 7, caspase 8, caspase 9, caspase 10, chymotrypsin, clostripain (clostridiopeptidase B), CNBr, factor Xa, formic acid, glutamyl endopeptidase, granzyme B, hydroxylamine (NH₂OH), iodosobenzoic acid, lys-C proteinase, NTCB+Ni (2-nitro-5-thiocyanobenzoic acid), pepsin, proline-endopeptidase, proteinase K, staphylococcal peptidase I, thermolysin, thrombin, and trypsin. In certain embodiments, it may be desirable to obtain two or more binding reagent-binding profiles for aliquots of a protein sample which have been separately digested with two or more proteolytic agents. When using two or more proteolytic agents, it may be desirable to select binding reagents that recognize epitopes produced using each of the proteolytic agents. Alternatively, a generic binding reagent set (e.g., no amino acids are fixed in the epitope to accommodate preferred ends produced by a particular proteolytic agent) may be used which is compatible with cleavage using any proteolytic agent.

After cleavage with a proteolytic agent, the peptide fragments may be tagged at the N-terminus or C-terminus using a tag or label suitable for detecting binding of the peptide fragments to the binding reagents. In certain embodiments, the label is a fluorescent tag, such as, for example, FAM, JOE, TET, HEX, TAMRA, Texas Red™, coumarin, rhodamine, PE, etc Cy3™, Cy5™, fluorescein and derivatives thereof, or other cyanine dyes such as the BODIPY™ or Alexa™ dyes which are commercially available from Molecular Probes, Inc. (www.probes.com). In an exemplary embodiment, the tag is fluorescein which has been shown to be an effective hapten in a hapten/carrier system (Colbert, D. L. et al., “The Effect of Fluorescein Labels on the Affinity of Antisera to Small Haptens,” J. Immunol. Methods, 140 (1991) 227-233, incorporated herein by reference in its entirety). In other embodiments, tags may be used, such as, without limitation, biotin, poly His tags, caged radionuclide structures for NMR and enzymes, such as alkaline phosphatase and horseradish peroxidase.

In still other embodiments, the binding reagents may be designed to recognize 1−n amino acids at the N-terminus or C-terminus of an un-modified peptide fragment (e.g., no tag or label attached to the N-terminus or C-terminus of the peptide fragments). In this embodiment, methods such as surface plasmon resonance may be used to detect binding of the peptide fragments to the binding reagents (for example, as described in U.S. Pat. Nos. 6,139,797 and 5,955,729, both of which are incorporated herein by reference in their entirety) and such as those systems commercially available from Biacore AB of Uppsala, Sweden.

In the various embodiments, the peptide fragments that are optionally tagged at either the N-terminus or the C-terminus of the fragments may be contacted with the solution set of discretely located binding reagents (discussed below). The conditions under which the binding reagents are contacted with the peptide fragments will naturally vary, depending on the nature of the binding assay. Typical antibody binding reaction conditions are well known in the art. The surface bearing the binding reagents typically is blocked to prevent non-specific binding reactions. One common binding and blocking solution used for ELISA assays is a solution of nonfat dry milk in Phosphate Buffered Saline (PBS). Washes may be conducted in a solution of PBS and Tween-20. The binding and washing of the surface of the substrate may be conducted using a variety of solutions, blocking agents, buffers, surfactants, viscosity modifiers, fluorescence enhancers, secondary indicators or enzymes and, optionally, substrates therefor, co-factors and the like, depending upon the binding and detection methods employed to detect binding of tagged peptide fragments to the binding reagents.

After contacting the peptide fragments with the binding reagents, the pattern of binding of the peptides to the solution set is determined and optionally compared to a database comprising information about the binding patterns for known proteins. In an exemplary embodiment, the peptide binding pattern for a protein from a given protein mixture will be compared to database comprising information about the binding pattern for each protein within the protein mixture from which the protein was isolated. The binding pattern for the proteins in a given protein mixture may be predicted based on the sequences of the proteins and the cleavage specificity of the proteolytic agent using, for example, the algorithms described below. Alternatively, the binding patterns for various proteins may be determined experimentally. The database of binding patterns for peptide fragments from known proteins may be stored in a computer readable format or may be contained in a printed document. The data may be stored and/or presented numerically or graphically. In an exemplary embodiment, the database is linked with the detection device used to determine the binding pattern of the peptide fragments to the binding reagent set and automatically identifies the protein in the assay.

Generation of antibodies may be accomplished by any number of well-known methods for generating monoclonal antibodies. These methods typically include the step of immunization of animals, typically mice, with a desired immunogen, for instance X₁ . . . Xₙ-Lys-Tag or Tag-Y₁ . . . Yₙ. Once the mice have been immunized, and preferably boosted one or more times with the desired immunogen(s), monoclonal antibody-producing hybridomas may be prepared and screened according to well known methods—(see, for example, Kuby, Janis, IMMUNOLOGY, Third Edition, pp. 131-139, W. H. Freeman & Co. (1997), for a general overview of monoclonal antibody production, that portion of which is incorporated herein by reference).

Over the past several decades, antibody production has become extremely robust. In vitro methods that combine antibody recognition and phage display techniques allow one to amplify and select antibodies with very specific binding capabilities. See, for example, Holt, L. J. et al., “The Use of Recombinant Antibodies in Proteomics,” Current Opinion in Biotechnology 2000, 11:445-449, incorporated herein by reference. These methods typically are much less cumbersome than preparation of hybridomas by traditional monoclonal antibody preparation methods. Binding epitopes may range in size from small organic compounds such as bromo uridine and phosphotyrosine to oligopeptides on the order of 7-9 amino acids in length. Examples of antibodies specific for di- and tri-peptides are described, for example, in Horibe, et al. Biochem. Biophys. Res. Comm. 281: 1321-1324 (2001). Thus, a desired set of antibody binding reagents that recognize epitopes of some fixed length may be generated readily using the methods described herein.

In one embodiment, employing phage display technology to generate binding reagents, an immune response to a selected immunogen or immunogens is elicited in a mouse and the response is boosted to expand the immunogen-specific B-cell population. Messenger RNA is isolated from those B-cells, or optionally a monoclonal or polyclonal hybridoma population. Animals other than mice may be used for the purposes herein. The mRNA is reverse-transcribed by known methods using either a poly-A primer or murine immunoglobulin-specific primer(s), typically specific to sequences adjacent to the desired V_{H} and V_{L} chains, to yield cDNA. The desired V_{H} and V_{L} chains are amplified by polymerase chain reaction (PCR) typically using V_{H} and V_{L} specific primer sets, and are ligated together, separated by a linker. V_{H} and V_{L} specific primer sets are commercially available, for instance from Stratagene, Inc. of La Jolla, Calif. Assembled V_{H}-linker-V_{L} product (encoding an scFv fragment) is selected for and amplified by PCR. Restriction sites are introduced into the ends of the V_{H}-linker-V_{L} product by PCR with primers including restriction sites and the scFv fragment is inserted into a suitable expression vector (typically a plasmid) for phage display. Other fragments, such as an Fab′ fragment, may be cloned into phage display vectors for surface expression on phage particles. The phage may be any phage, such as lambda, but typically is a filamentous phage, such as fd and M13, typically M13.

In phage display vectors, the V_{H}-linker-V_{L} sequence is cloned into a phage surface protein (for M13, the surface proteins g3p (pIII) or g8p, most typically g3p). Phage display systems also include phagemid systems, which are based on a phagemid plasmid vector containing the phage surface protein genes (for example, g3p and g8p of M13) and the phage origin of replication. To produce phage particles, cells containing the phagemid are rescued with helper phage providing the remaining proteins needed for the generation of phage. Only the phagemid vector is packaged in the resulting phage particles because replication of the phagemid is grossly favored over replication of the helper phage DNA. Phagemid packaging systems for production of antibodies are commercially available. One example of a commercially available phagemid packaging system that also permits production of soluble ScFv fragments in bacteria cells is the Recombinant Phage Antibody System (RPAS), commercially available from Amersham Pharmacia Biotech, Inc. of Piscataway, N.J. and the pSKAN Phagemid Display System, commercially available from MoBiTec, LLC of Marco Island, Fla. Phage display systems, their construction and screening methods are described in detail in, among others, U.S. Pat. Nos. 5,702,892, 5,750,373, 5,821,047 and 6,127,132, each of which are incorporated herein by reference in their entirety.

Typically, once phage are produced that display a desired antibody fragment, epitope-specific phage are selected by their affinity for the desired immunogen and, optionally, their lack of affinity to compounds containing certain other structural features. A variety of methods may be used for physically separating immunogen-binding phage from non-binding phage. Typically the immunogen is fixed to a surface and the phage are contacted with the surface. Non-binding phage are washed away while binding phage remain bound. Bound phage are later eluted and are used to re-infect cells to amplify the selected species. A number of rounds of affinity selection typically are used, often increasingly higher stringency washes, to amplify immunogen-binding phage of increasing affinity. Negative selection techniques also may be used to select for lack of binding to a desired compound. In that case, un-bound (washed) phage are amplified.

The binding reagents do not have to originate from biological sources, such as from naive or immunized immune cells of animals or humans. The binding reagents may be screened from a combinatorial library of synthetic peptides. One such method is described in U.S. Pat. No. 5,948,635, incorporated herein by reference, which described the production of phagemid libraries having random amino acid insertions in the pIII gene of M13. These phage may be clonally amplified by affinity selection as described above.

The binding reagents also may be aptamers, oligonucleotides that are selected to bind specifically to a desired molecular structure. Aptamers typically are the products of an affinity selection process similar to the affinity selection of phage display (also known as in vitro molecular evolution). The process involves performing several tandem iteratations of affinity separation, e.g., using a solid support to which the desired immunogen is bound, followed by polymerase chain reaction (PCR) to amplify nucleic acids that bound to the immunogens. Each round of affinity separation thus enriches the nucleic acid population for molecules that successfully bind the desired immunogen. In this manner, a random pool of nucleic acids may be “educated” to yield aptamers that specifically bind target molecules. Aptamers typically are RNA, but may be DNA or analogs or derivatives thereof, such as, without limitation, peptide nucleic acids (PNAs) and phosphorothioate nucleic acids.

Although it is preferred to use spleen cells and/or B-lymphocytes from animals pre-immunized with a desired immunogen as a source of cDNA from which the sequences of the V_{H} and V_{L} chains are amplified by RT-PCR, naive (un-immunized with the target immunogen) splenocytes and/or B-cells may be used as a source of cDNA to produce a polyclonal set of V_{H} and V_{L} chains that are selected in vitro by affinity, typically by the above-described phage display (phagemid) method. When naive B-cells are used, during affinity selection, the washing of the first selection step typically is of very low stringency so as to avoid loss of any single clone that may be present in very low copy number in the polyclonal phage library. By this naive method, B-cells may be obtained from any polyclonal source. B-cell or splenocyte cDNA libraries also are a source of cDNA from which the V_{H} and V_{L} chains may be amplified. For example, suitable murine and human B-cell, lymphocyte and splenocyte cDNA libraries are commercially available from Stratagene, Inc. and from Clontech Laboratories, Inc. of Palo Alto, Calif. Phagemid antibody libraries and related screening services are provided commercially by Cambridge Antibody Technology of the U.K. or MorphoSys USA, Inc., of Charlotte, N.C.

The immunogens described herein typically are a fluorescent dye molecule covalently bound to a peptide chain of from to about 10 amino acids, forming a hapten which is linked to a carrier or support, such as a bead. The desired binding reagents will bind an epitope that includes the peptide chain in combination with the tag. The epitope includes the peptide chain and, optionally, portions of the tag. It is not important that the epitope includes the tag or portions thereof, but that the affinity of the binding reagent is substantially increased (detectably by any assay used to detect binding of the binding reagent to the peptide chains) by the presence of the tag adjacent to the peptide, whether or not the epitope contains portions of the tag. Thus, the increased affinity may be due to, for instance and without limitation, steric effects or other proximity effects the tag may have on the peptide.

The N-terminal tagged immunogen has a structure Tag-X₁- . . . Xₙ-L-C, where L is optional and is a linker and C is a carrier. C may be a functionalized bead as is typically used in immunizations, such as SASRIN™ resin commercially available from Bachem, King of Prussia, Pa. C may be a carrier protein typically used for immunizations, such as keyhole limpet hemocyanin (KLH) or bovine serum albumin (BSA). The linker L may a non-immunogenic synthetic linker, such as a polyethylene glycol (PEG) residue, amino caproic acid or derivatives thereof. L also may be a random, or semi-random polypeptide. Since the residues Ser, Glu and Leu typically follow trypsin cleavage sites (90% of the time), it may be preferred that at least the first residue of the linker adjacent Xₙ is randomly selected from Ser, Glu and Leu. This immunogen may be synthesized by standard methods, such as by 1-ethyl-3-[dimethylaminopropyl] carbodiimide (EDC)-catalyzed condensation of amine and carboxyl groups. Peptide chains are synthesized by standard methods in a C-terminal to N-terminal direction either on the carrier bead (optionally functionalized with linker L) or a functionalized surface, from which the peptide is cleaved by standard methods. In any case, the chemistries by which the various units (Tag, X₁₋ₙ, L and C) may be synthesized and assembled are mature technologies and may be accomplished by any method known in the art. The C-terminal tagged immunogen has a structure C-L-X₁- . . . Xₙ-Tag, and, as with the N-terminal tagged immunogen, may be synthesized by any method known.

If immunization of animals (mice) is used as a step in the preparation of binding reagents, KLH may be a preferred carrier in that, in mice, it typically elicits the most robust immune response to haptens. Nevertheless, a strong, matured immune response may not be necessary, or preferred in the case where phage display is used as the method to clonally expand the desired binding reagents. A strong, mature immune response might yield very specific binding reagents that may not have the desired specificities. Thus, immunization may also be carried using a bead-hapten conjugate which is relatively easy to synthesize but which typically does not elicit as strong an immune response as KLH-hapten conjugates in mice.

Panning in a culture dish or flask is one way to physically separate binding phage from non-binding phage. Panning may be carried out in 96 well plates in which desired peptide/tag immunogen structures may be synthesized. Functionalized (NH₂ or COOH) 96 well plates, typically used as ELISA plates, may be purchased from Pierce of Rockwell, Ill. Peptides may be synthesized in an N-terminal to C-terminal direction, or vice-versa, by standard methods and the tag also may be attached by standard methods, such as by an EDC-catalyzed condensation of an amine and a carboxyl group. Other affinity methods include affixing the immunogen to beads. The beads may be placed in a column and phage may be bound to the column, washed and eluted according to standard procedures. Alternatively, the beads may be magnetic so as to permit magnetic separation of the binding particles from the non-binding particles. The immunogen also may be affixed to a porous membrane or matrix, permitting easy washing and elution of the binding phage.

Because it is desired that the binding reagent bind only to tagged ends of the peptide fragments, the affinity selection process for the binding reagents may include a negative selection step for binding reagents that do not bind to the target peptide when the peptide is not conjugated to the tag. In one example, trypsin-digested yeast total protein is affixed to a surface, such as a porous matrix and binding reagent-displaying phage are absorbed on the surface. Digested total protein from any given protein mixture may be used, and, in the case where the binding reagent set is tailored for a given protein mixture, or the binding reagents are to be used to analyze proteins from a given protein mixture, the digested total protein may be prepared from a sample of the appropriate protein mixture. Random synthetic peptides also may be attached to the surface to absorb the phage. Phage are washed from the surface and non-binding phage are grown to clonally expand the population of non-binding phage, thereby de-selecting phage that do not require the tag to bind to the target peptide(s).

Screening of binding reagents will best be accomplished by high throughput parallel selection, as described in Holt et al. In certain embodiment, for example the generic sets described above, where absolute specificity for an epitope is desired, but not absolutely required, a chip with a generic C (2, 2) array may be used for high throughput screening. As used herein, an array may be referred to by the shorthand C(i, j), where i is the number of amino acids in an eptitope adjacent to the tagged C-terminal amino acid and j is the number of amino acids in an epitope adjacent an N-terminal tag. Thus, a C(2,2) chip contains binding reagents specific to epitopes -X1-X2-Tag and Tag-Y₁-Y2-. In other embodiments, where specificity of the binding reagent set is required, a given screening array may include the desired epitope as well as near cognates of the desired epitopes. By “near cognates” it is meant peptide-Tag combinations with conservative amino acid substitutions, including but not limited to the conservative substitution groups such as: (i) a charged group, consisting of Glu and Asp, Lys, Arg and His, (ii) a positively-charged group, consisting of Lys, Arg and His, (iii) a negatively-charged group, consisting of Glu and Asp, (iv) an aromatic group, consisting of Phe, Tyr and Trp, (v) a nitrogen ring group, consisting of His and Trp, (vi) a large aliphatic nonpolar group, consisting of Val, Leu and Ile, (vii) a slightly-polar group, consisting of Met and Cys,(viii) a small-residue group, consisting of Ser, Thr, Asp, Asn, Gly, Ala, Glu, Gln and Pro, (ix) an aliphatic group consisting of Val, Leu, Ile, Met and Cys, and (x) a small hydroxyl group consisting of Ser and Thr. Conservative substitutions also may be determined by one or more methods, such as those used by the BLAST (Basic Local Alignment Search Tool) algorithm, such as a BLOSUM Substitution Scoring Matrix, such as the BLOSUM 62 matrix, and the like. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). Alternatively, high throughput parallel selection may be conducted by commercial entities, such as by Cambridge Antibody Technologies or MorphoSys USA, Inc.

Alternatively, selection of a desired binding reagent-displaying phage may be carried out using the following method:

Step 1: Affinity purify phage under low stringency conditions for their ability to bind to an immunogen fixed to a solid support (for instance, beads in a column).

Step 2: Elute the bound phage and grow the eluted phage. Steps 1 and 2 may be repeated with more stringent washes in Step 1.

Step 3: Absorb the phage under moderate stringency with a given protein mixture digested with a proteolytic agent of interest. Wash away the unbound phage with a moderately stringent wash and grow the washed phage. Step 3 may be repeated with less stringent washes.

Step 4: Affinity purify phage under high stringency for their ability to bind to the immunogen fixed to a solid support. Elute the bound phage and grow the eluted phage.

Step 5: Plate the phage to select single plaques. Independently grow phage selected from each plaque and confirm the specificity to the desired immunogen.

This is a general guideline for the clonal expansion of immunogen-specific binding reagents. Additional steps of varying stringency may be added at any stage to optimize the selection process, or steps may be omitted or re-ordered. One or more steps may be added where the phage population is selected for its inability to bind to other tag-conjugated immunogens in the binding reagent set by absorption of the phage population with those other tag-conjugated immunogens and amplification of the unbound phage population. That step may be performed at any stage, but typically would be performed after step 4.

In certain embodiments, it may be desirable to mutate the binding region of the binding reagent and select for binding reagents with superior binding characteristics as compared to the un-mutated binding reagent. This may be accomplished by any standard mutagenesis technique, such as by PCR with Taq polymerase under conditions that cause errors. In such a case, the PCR primers could be used to amplify scFv-encoding sequences of phagemid plasmids under conditions that would cause mutations. The PCR product may then be cloned into a phagemid vector and screened for the desired specificity, as described above.

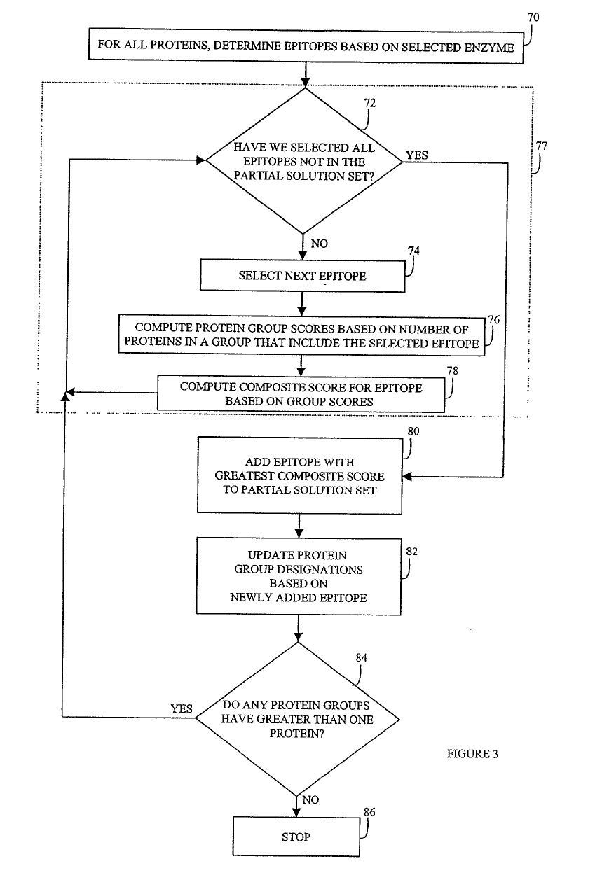

In another embodiment, once a full set of binding reagents is produced, capable of distinguishing most members of a protein mixture, the binding reagents are affixed to one or more supports at discrete locations (that is, binding reagents having a first specificity are affixed at a first spatial location, binding reagents having a second specificity are affixed at a second spatial location, etc.). In one embodiment, the binding reagents are affixed to a substrate in a tiled array, with each binding reagent represented in one or more positions in the tiled array. Protein microarrays are described in PCT Publication WO 00/04389, incorporated herein by reference. Examples of commercially available protein microarrays are those of Zyomyx of Hayward, Calif., Ciphergen Biosystems, Inc. of Fremont, Calif. and Nanogen, Inc. of San Diego, Calif. The spatial configuration of the substrate or substrates may be varied so long as each binding reagent species is bound at detectably discrete locations. The substrate and tiled binding reagent pattern typically is planar, but may be any geometric configuration desired. For instance, the substrate may be a strip or cylindrical, as illustrated in U.S. Pat. No. 6,057,100, FIGS. 3A-3E. The substrate typically is glass or other silicic compositions, such as those used in the semiconductor industry. Fabrication of the substrate may be by one of many well-known processes.

The binding reagents (for example, phage, antibodies, antibody fragments, aptamers, etc.) may be affixed to a suitable substrate by a number of known methods. Typically the surface of the substrate is functionalized in some manner, so that a crosslinking compound or compounds may covalently link the binding reagent to the substrate. For example, a substrate functionalized with carboxyl groups may be linked to free amines in the binding reagents using EDC or by other common chemistries, such as by linking with N-hydroxysuccinimide. A variety of crosslinking chemistries are commercially available, for instance, from Pierce of Rockford, Ill.

In another embodiment, each binding reagent species is bound to discrete beads. For instance, each binding reagent species is reacted with a different color-coded microsphere, such as the Luminex Microspheres commercially available from Luminex Corporation of Austin, Tex. That method is described in detail in U.S. Pat. No. 5,981,180, which is incorporated herein by reference in its entirety. The microspheres are internally dyed polystyrene microspheres dyed with two spectrally distinct fluorochromes. By using precise ratios of the two fluorochromes, a spectral array is created, each microsphere having different ratios of the two fluorochromes, and therefore having a different spectral address. Although currently limited to about 100 different spectral addresses, the use of different fluorochromes would increase this number. Even if only 100 spectral addresses are available, the assay may be run in multiple stages with different binding reagents on beads having the same spectral address at each stage. For instance, eight different binding reagents may be bound to beads having the same spectral address. So long as these eight beads are kept separate and are analyzed separately, they may be used to determine binding to the eight different binding reagents. Due to the high throughput nature of this assay, the eight different samples may be analyzed quite quickly. In this manner, binding to any multiple of the 100 (spectral addresses) binding reagents may be determined.

The microspheres typically are either carboxylated or avidin-modified so that proteins, such as antibodies, fragments thereof, phage or other binding reagents may be readily attached to the beads by standard chemistries. In one example, the binding reagents are scFv fragments. The scFv fragments may be bound to carboxylated beads by one of many linking chemistries, such as, for example, EDC chemistry, or bound to avidin-coated beads by first biotinylating the scFv fragment by one of many common biotinylation chemistries, such as, for example, by conjugation with sulfo-NHS-LC-biotin (Pierce).

Once bound to the discretely colored beads, the binding reagents may be reacted with a protein sample cleaved into peptide fragments using a proteolytic agent and Tag-conjugated at either the N-terminus or the C-terminus of the peptide fragments. The tag may be any molecule suitable for detection of the peptide fragments. In certain embodiments, the tags are fluorescent, such as, for example, Cy3™, Cy5™, fluorescein, or derivatives thereof, or the Alexa™ or BODIPY™ dyes from Molecular Probes, Inc. of Eugene, Oreg. The beads are then laser-scanned twice, first, to determine the color of the bead, thereby identifying the binding reagent species bound to the bead, and then to determine whether the binding reagent has bound Tag-conjugated peptide. Scanners and scanning systems having this capability are commercially available from Luminex Corporation, permitting the evaluation of thousands of coated beads per second.

Those of ordinary skill will recognize that peptide fragments produced from a protein cleaved with a proteolytic agent may bind to a subset of binding reagents in the binding reagent set. Disclosed herein is a method and system for determining a an optimum set of epitopes (“solution set”) to which a set of binding reagents may be designed for identifying proteins from a protein mixture. Although the illustrated embodiments include an example that utilizes purified proteins from yeast (Saccharomyces cerevisiae) and bacteria (E.Coli), those with ordinary skill in the art will recognize that such an example is provided merely for illustration and not limitation, and other protein mixtures (e.g., from a given organism, proteome, organ, tissue, cell, organelle, or sub-cellular localization, or portion thereof) may be substituted and/or used in addition to the illustrated example, without departing from the scope of the disclosed methods and systems.

The disclosed methods and systems can utilize combinatorial optimization to select, define, identify, designate, or otherwise choose (hereinafter referred to collectively as “identify”) a solution set of epitopes that can be associated with a set of binding reagents to identify proteins, where such proteins can be part of a group or set of proteins otherwise referred to herein as a protein mixture or catalog. This selection or identification process can also be described as choosing the minimum number of binding reagents or antibodies (“selected set”) based on varying constraints, such that the binding reagent binding pattern for a given protein is unique for each protein to be identified and/or distinguished.

A solution to the protein identification problem can thus provide for each possible protein pair (i.e., each combination of two proteins) in the protein catalog, a binding reagent that binds to one of the proteins in the pair, but not to the other protein in that pair. This problem can otherwise be known as the test collection problem, and can be viewed as a combinatorial optimization problem. For example, a yeast proteome that provides a catalog of approximately six thousand proteins can translate to approximately eighteen-million unique protein pairs for which a comparison of “bind to one protein in the pair, but not to the other,” can be posed. Because a problem of this size causes prohibitive storage requirements, constraints can be provided to optimize the solution.

The disclosed methods and systems identify at least one opitimal set of binding reagents by identifying at least one optimal set of distinguishing epitopes. The solutions sets of distinguishing epitopes can then be associated with corresponding binding reagents. As provided previously herein, the term epitope can be understood more generally as a peptide fragment.

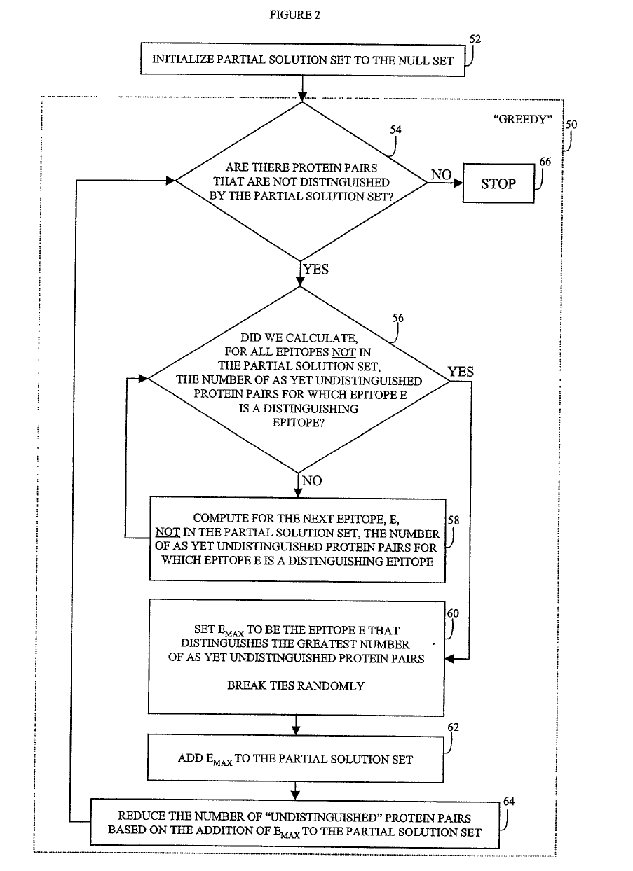

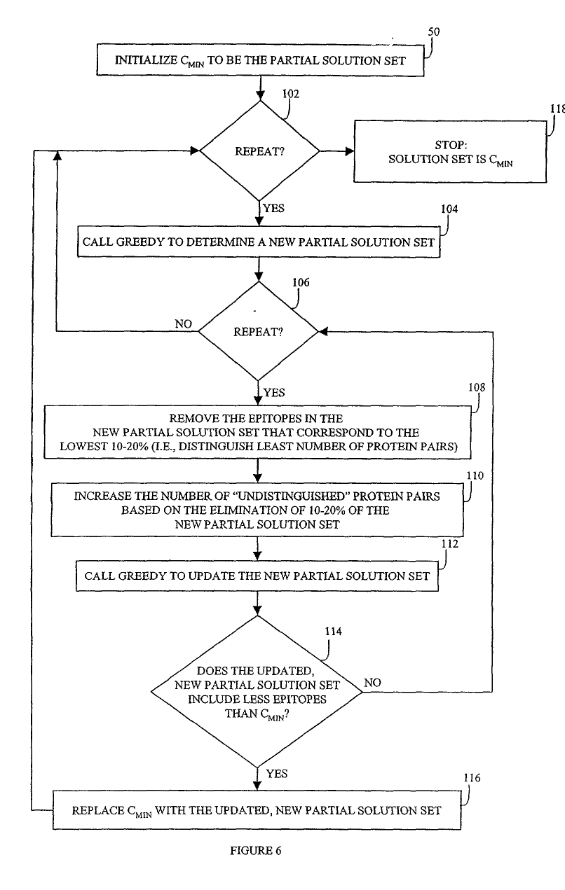

In one embodiment, a randomized greedy algorithm with local search can be employed to identify a solution set of epitopes, which in some embodiments can be a minimum set of epitopes, to identify and/or distinguish proteins in a protein catalog. In a randomized greedy algorithm, the solution set can be generated incrementally as provided in FIG. 2. As will be shown in FIG. 6, the solution set (or “partial solution set”) identified by the randomized greedy algorithm can be further enhanced with a local search. Those with ordinary skill in the art will recognize that the local search can enhance a solution that may have settled in a local minimum. Accordingly, for the methods and systems disclosed herein, a minimum-size solution set of epitopes (and/or antibodies) may not be unique for a given protein catalog, and hence different, minimum-size solution sets for the same protein catalog can exist, where the minimum size is the same, but the epitopes within the different solution sets can be different. Furthermore, multiple solutions of larger-sized solution sets can also exist.

For the purposes of the methods and systems herein, a protein can be understood to “include” an epitope when, for a given proteolytic agent, the protein can be cleaved into peptide fragments wherein at least of the fragments include the epitope.

FIG. 2 provides a method for determining a solution set, otherwise referred to as a partial solution set, that can include a minimum number of epitopes or binding reagents using a randomized greedy algorithm 50. Initially, the partial solution set does not include any epitopes, and hence the partial solution set can be viewed as the null set 52. In decision 54, it is determined whether there are any protein pairs that are not distinguished by the presently existing partial solution set. Initially, when the partial solution set is null, there are not any protein pairs that are distinguished, and hence the algorithm proceeds to 56.

Decision 56 and block 58 include an iterative selection of epitopes that are not presently in the partial solution set. For epitopes not in the partial solution set, block 58 includes determining, for a given epitope, the number of protein pairs that can be distinguished by the given epitope. Once this determination is made for the epitopes not in the partial solution set, the algorithm proceeds to block 60 that includes determining which of the epitopes (not in the partial solution set) distinguishes the most, previously undistinguished protein pairs. If two or more epitopes are equally efficient at distinguishing protein pairs, a “tie” or equivalence exists, and the equivalence can be resolved by randomly choosing one of the equally efficient epitopes as the most effective epitope. The most effective epitope, designated Emax, and which can be referred to as the “maximum epitope,” can be added to the partial solution set 62. With Emax as part of the partial solution set, the number of previously undistinguished protein pairs can be reduced by the number of protein pairs distinguished by Emax 64. The algorithm then returns to 54, where it is determined whether any undistinguished protein pairs exist. When no undistinguished protein pairs exist, the algorithm is complete 66. In one embodiment, at this time, the partial solution set of epitopes can be associated with binding reagents to form an array for protein identification.

As previously provided herein, if the FIG. 2 algorithm is applied to the yeast proteome that includes approximately six thousand proteins, the number of initially undistinguished protein pairs approximates nearly eighteen million. Because computations of this magnitude present large storage and processor constraints, a simplified method is provided in FIG. 3.

FIG. 3 provides additional and/or optional detail for certain features of FIG. 2, while generally presenting an illustration of methods and systems that are equivalent to the methods and systems of FIG. 2. For example, as FIG. 3 indicates, during preprocessing, epitopes for the proteins in a protein catalog can be determined (“determined epitopes”) based on a selected proteolytic agent and/or enzyme 70. In the yeast proteome example, a database can be compiled to include the peptide fragments and hence epitopes for the approximately six thousand yeast proteome proteins for a given proteolytic agent. The epitopes not within the partial solution set (i.e., initially, all known epitopes in all the known proteins) can thus be selected one at a time 72, 74 and a group score can be computed for presently existing protein groups 76.

A protein group includes proteins that are presently undistinguished by the current partial solution set, while proteins within different groups can be distinguished by the current partial solution set. Accordingly, to be undistinguished implies that for each given epitope within the solution set, all known proteins in a given protein group either include the given epitope, or all known proteins in that same given protein group do not include the given epitope. For the example of the yeast proteome, during the first iteration for a method and system according to FIG. 3, the partial solution set is the null set, and there can thus be one protein group that includes approximately six thousand proteins. At a given time, a protein in the protein catalog thus can be associated with other proteins and/or belong to exactly one protein group. For explanatory purposes, associated proteins can be referred to herein as protein groups. All determined epitopes can then be selected, one at a time 72, 74 and compared to the proteins in the single protein group. The epitope that is included in the most proteins can be designated Emax (FIG. 2) or the maximum epitope, and Emax can be added to the partial solution set (i.e., “identified epitopes”) and also removed from the “determined epitope” list (i.e, the list of all known epitopes in all known proteins in the protein catalog, respective to the proteolytic agent). The single protein group can thus be divided into two protein groups based on the presence of Emax in the partial solution set: the first protein group includes proteins that include Emax, and the other protein group includes proteins that do not include Emax.

Subsequent iterations of the epitope selection loop 77 seek to determine which of the remaining “determined epitopes” can further divide the greatest number of presently existing protein groups based on the existence of the epitope in at least one, but less than all of the proteins in a given protein group. Accordingly, a group score for the protein groups can be determined, and the group scores can be combined to form a composite group score 78. In one embodiment, the epitope with the largest composite group score can be understood to be Emax and added to the partial solution set 80 and removed from the “determined epitope” list. As previously provided herein, the protein groups can be redistributed (i.e., divided) based on the inclusion of the new epitope (i.e., Emax) in the partial solution set. The new protein groups can then be assessed to determine if a protein group includes more than one protein 84. If all protein groups include one protein, the partial solution set is complete 86, and in some embodiments, an array can be formed based on the epitopes in the partial solution set. If all protein groups do not include one protein, a method and system according to FIG. 3 can continue at 72 by cycling through the remaining “determined epitopes” 77 to find the next Emax that can divide the most protein groups.

In one embodiment of a method and system according to FIGS. 2 and/or 3, a protein group score can be designated a number equal to the number of occurrences of the epitope in the protein group, multiplied by a quantity expressed as the difference between the size of the group less the number of occurrences of the epitope in the protein group (i.e, (number of occurrences in group)*(size of group−number of occurrences in group)). The composite score can include an accumulation of the group scores. In some embodiments, protein groups that include only one protein (i.e., a protein is unassociated with another protein) may not be given a group score, while in other embodiments, a protein group that includes only one protein can be given a score of zero, or some other score.

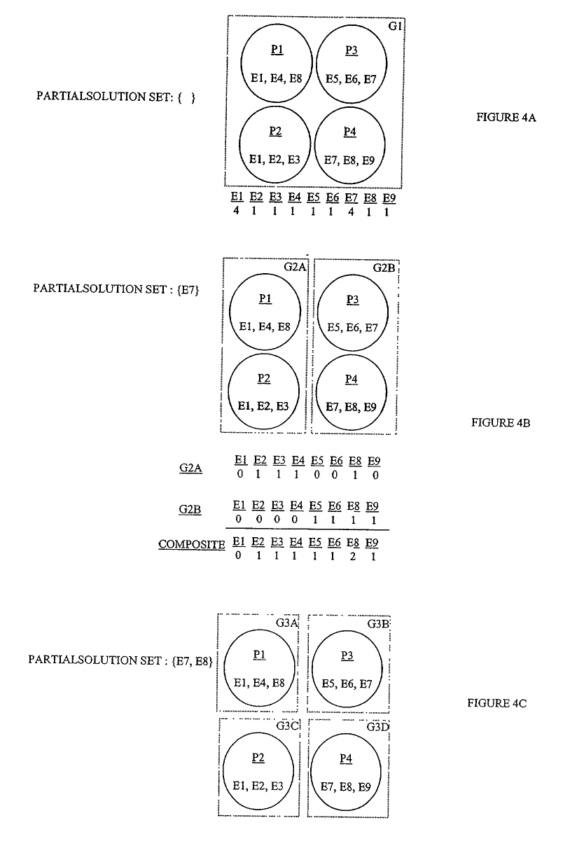

FIGS. 4A, 4B, and 4C provide an exemplary embodiment for a system and method according to FIGS. 2 and/or 3 that includes a protein catalog of four (4) proteins designated P1, P2, P3, and P4. The four proteins in the protein catalog are also depicted with their respective epitopes (E1-E9) based on cleavage by one or more proteolytic agents. For example, P1 includes E1, E4, and E8. As shown in FIG. 4A, initially, the partial solution set does not include any epitopes, and hence, there is one protein group (G1) that includes the four proteins of the protein catalog. The first epitope to become part of the partial solution set can be determined in accordance with FIGS. 2 and/or 3 by determining, for all “determined epitopes,” that epitope which divides the single group, G1. When there is a single group in the first iteration, a “composite” group score can be obtained by merely counting the number of occurrences of the determined epitopes in the proteins. FIG. 4A indicates that epitopes E1 and E7 have a composite group score of two because E1 and E7 appear in two of the four proteins, while epitopes E2-E6 and E8-E9 have a composite group score of one because these epitopes appear in a single protein. Because epitopes E1 and E7 are equally effective at partitioning G1, the embodiment of FIG. 4A can randomly select E7 as Emax.

FIG. 4B represents a second iteration of a method and system according to FIGS. 2 and/or 3, and as FIG. 4B indicates, the partial solution set includes E7, and thus the exemplary list of “determined epitopes” is reduced to E1-E6 and E8-E9. Further, G1 is partitioned or divided by designating those proteins that include epitope E7 as belonging to group “G2B”, and designating those proteins that do not include epitope E7 as belonging to group “G2A”. In accordance with FIGS. 2 and 3, the remaining determined epitopes (E1-E6, E8-E9) can be scored on a protein group basis. FIG. 4B provides the scores for groups G2A and G2B for the eight remaining determined epitopes. The FIG. 4B embodiment linearly combines the various protein group scores by adding the protein group scores to provide a composite group score. As FIG. 4B indicates, epitope E8 has the largest composite group score, and hence E8 is the new Emax.

Referring to FIG. 4C, epitope E8 is added to the partial solution set, and groups G2A and G2B are separately partitioned based on the occurrence/non-occurrence of epitope E8. As FIG. 4C indicates, this partitioning provides four protein groups (G3A, G3B, G3C, and G3D) that include one protein. Because all protein groups include a single protein, a partial solution set is determined.

Those with ordinary skill in the art will recognize that the embodiments of FIGS. 2 and 3, and the illustrations of FIGS. 4A-4C, can be accomplished in many ways. As the FIGS. 4A-4C illustrations indicate, different scoring techniques can be used for different phases (iterations) of the disclosed methods and systems. Those with ordinary skill in the art will also recognize that the methods and systems can be facilitated using a database or other type of organized electronic storage that can facilitate associating proteins with epitopes, and associating a protein group (e.g., label or other designation) with a protein. In embodiments that use a database, various database querying techniques can be used to identify protein groups and generate protein group scores, combine protein group scores to formulate a composite group score, etc. In other embodiments, linked lists, trees, queues, hash tables, or other data structures can be used to organize protein groups and otherwise facilitate processor/computer processing in accordance with the disclosed methods and systems. In some embodiments, accordingly, protein groups may not be labeled, and can otherwise be stored in a manner that facilitates group recognition by associating proteins. Accordingly, a protein group as provided herein is a mere association of proteins, and the methods and systems disclosed herein can continue until the proteins in the protein catalog are unassociated with another protein in the protein catalog. Similarly, those with ordinary skill in the art will recognize that the choice of scoring is merely arbitrary, and other scoring mechanisms can be used without departing from the scope of the methods and systems. For example, a scaled scoring can be used where in the actual number of proteins within a protein group can affect the score. In another embodiment, a binary scoring scheme can be used. Another example can include an entropy scoring scheme wherein a group score can be equal to its fraction of the catalog size multiplied by the logarithm of this number. See also B. M. E. Moret and H. D. Shapiro, “On minimizing a set of tests”, SIAM J. Scientific & Statistical Computing 6 (1985), 983-1003, incorporated herein by reference in its entirety.

When the methods and systems of FIGS. 3 and 4A-4C are applied to the yeast proteome example, when a new epitope is selected 74 and evaluated 76, the evaluation includes on the order of six thousand comparisons (i.e., the number of proteins in the protein catalog) to determine the new Emax. This computation is markedly more efficient than the approximately eighteen million computations that can be required as previously provided herein.

The methods and systems of FIGS. 3 and 4A-4C also facilitate a method and system for forming a unique representation for proteins in the protein catalog. As previously provided herein, the partial solution set can be incrementally determined by iteratively selecting an epitope (Emax) from the remaining set of “determined epitopes,” where Emax causes the greatest number of protein groups to be divided. In some embodiments, when a new Emax is selected and the partial solution set increases in size, a binary representation can also be incrementally formed or otherwise augmented to provide a “bar code” that uniquely represents the proteins in the protein catalog.

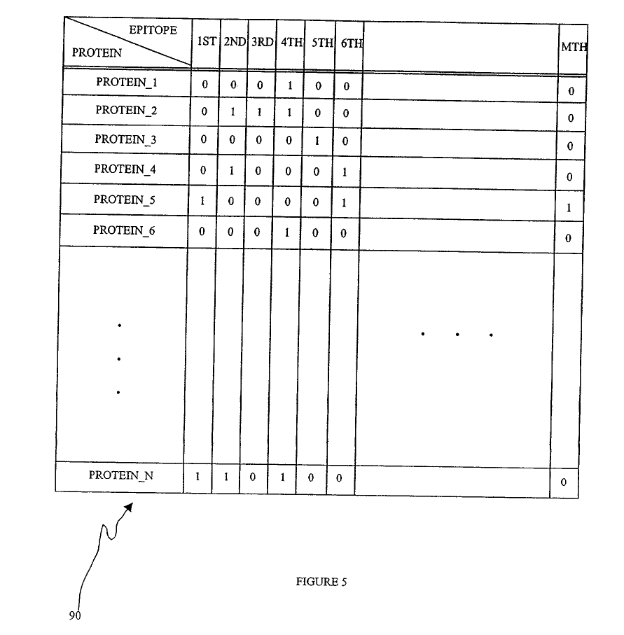

FIG. 5 provides one illustration that indicates the binary representation, or in an embodiment, “bar code”, that can be generated for proteins in the protein catalog based on the epitopes in the partial solution set. The FIG. 5 rows provide the N proteins in the protein catalog, while the FIG. 5 columns represent the M epitopes in the partial solution set. For epitopes in the partial solution set, a protein in the protein catalog can be assigned a zero or a one based on the presence of the epitope in the protein (based on the proteolytic agent). In one embodiment, for example, when an epitope is added to the partial solution set, the binary representation for proteins in the protein catalog can be updated with a “one” if the epitope is included in the protein, and a “zero” otherwise. Alternately, an opposite assignment can be made. The updating of the bar code can occur incrementally during the generation of the solution set, or the bar code or other representation can be formed after the entire partial solution set is formed, or at intermediate points therein.

Referring back to FIGS. 4A-4C, for example, a binary representation for P1 based on the partial solution set of {E7, E8} can be “01”, while the representations for P2 can by “00”, for P3, “10”, and for P4, “11.” As indicated herein, the binary representation for the proteins are unique to the partial solution set. Similarly, one of ordinary skill in the art will recognize that there can be multiple partial solution sets for a given protein catalog, and the exemplary protein catalog of FIGS. 4A-4C is one such protein catalog. Recall the discussion relative to FIG. 4A, when epitope E7 was randomly chosen because epitope E1 was equally efficient. Had epitope E1 been randomly selected, the partial solution set could include E1 and E8, and hence the binary representations for P1-P4 would include “11”, “10”, “00”, and “01”, respectively.

The assignment of a binary code and/or bar code to the proteins in the protein catalog can facilitate efficient processing during protein identification, and during the local search. FIG. 6 provides one embodiment of a greedy algorithm with local search that can be employed with the greedy algorithm described in FIGS. 2-4C. Those of ordinary skill in the art will recognize that the local search can improve the performance of the greedy algorithm by attempting to prevent a partial solution set that resides in a local minimum. Accordingly, the methods and systems of FIG. 6 begin with assigning the partial solution set to be Cmin 100. It can be understood that the partial solution set assigned to Cmin in 100 was processed previously using the methods and systems of FIG. 6. The repeat of 102 determines the number of times that a greedy algorithm, including an algorithm according to FIGS. 2-4C, can be performed. As indicated herein, a partial solution set may not be unique, hence repeating a greedy algorithm 104 multiple times using the same protein catalog, proteolytic agent(s), and epitopes, can provide multiple partial solution sets. Alternately, the repeat of 106 can determine the number of times that a local search can be applied to a particular solution set. The repeats 102, 106 can be, and are likely different values, and can be determined based on desired performance and/or computational constraints (protein size, memory, processor speed, expected partial solution set size, etc.). In some embodiments, the repeat of 106 can be a larger number than the repeat of 102. Because the repeats 102, 106 are application dependent and can be performed as many times as desired, repeat values are not specified in FIG. 6.

Once the greedy algorithm is performed 104 and a partial solution set exists, a local search can be applied by eliminating at least some of the epitopes from the partial solution set 108. In the illustrated embodiment, the removed epitopes can include the least effective epitopes, where such epitopes can be determined based on a small relative composite group score (e.g., based on percent of protein groups that the epitope divided). In other embodiments, the removed epitopes can be randomly removed. Those with ordinary skill in the art will recognize that there are different criteria that can determine which epitopes to remove, and the methods and systems disclosed herein are not limited by the method of determining epitope removal.

Once an epitope is removed, at least some previously “distinguished” protein pairs are likely to not be distinguished any longer. With reference to the methods and systems of FIGS. 2-4C, when protein pairs can be viewed as protein groupings based on epitopes, the removal of an epitope from a partial solution set can cause one or more protein groups to recombine or regroup. Accordingly, to facilitate a regrouping based on epitope removal from the solution set, in an embodiment of the methods and systems, during the greedy algorithm, when an epitope is added to the partial solution set, the incidence with all the sets and the updated partial solution set is converted to a bit value and additionally and optionally can be further encoded using a hash table. In such an embodiment, regrouping based on epitope removal can be facilitated by examining the sets that are incident to the removed epitope and identifying those sets that will merge. Although FIG. 6 indicates that between ten and twenty percent of the epitopes can be removed, such percentage is merely illustrative and not limiting, and the percent and/or number of epitopes removed can vary and depend on the application.

Once the desired number of epitopes are removed from the partial solution set 108 and appropriate protein groups are reassembled (regrouped) 110, the greedy algorithm can be employed with the partial solution set (i.e., some epitopes removed) and the regroupings 112 to form an updated partial solution set. This updated partial solution set can be compared with Cmin to determine which partial solution set includes the smaller number of epitopes. As shown by FIG. 6, this process can repeat 102 as desired to provide a minimal sized epitope solution set that can be used to determine binding reagents and produce an array for protein identification.

Those of ordinary skill in the art will recognize that the disclosed methods and systems can be used with additional information to improve the efficiency and/or effectiveness of the systems and methods. For example, molecular weight can be used to distinguish proteins in the protein catalog, and the disclosed methods and systems can be reserved for distinguishing protein pairs in the protein catalog that cannot be distinguished by molecular weight, for example. In some embodiments, proteins in the protein catalog can be divided into sub-catalogs based on molecular weight, wherein the methods and systems can be applied to the sub-catalogs to distinguish proteins in a subcatalog. In an embodiment, a solution set can be determined for respective subcatalogs. Other additional information can also be used in combination with the disclosed methods and systems to enhance protein identification and performance of the disclosed methods and systems.

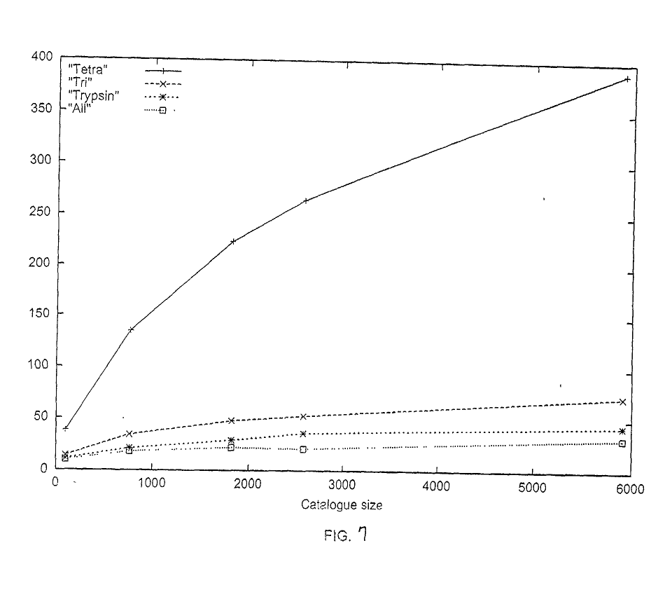

In some embodiments, protein catalogs can be pruned to proteins where the total difference in amino acid composition was no more than ten. For the illustrated results of FIG. 7 that includes utilizing the disclosed methods and systems to determine a minimum number of epitopes for the various conditions and/or constraints, proteins in the yeast protein catalog were cleaved with the enzyme trypsin that cleaves at lysines and arginines that are not followed by a proline. Proteins with fewer than five trypsin cleavage sites or less than one hundred amino acids were discarded. These pruning rules reduced the number of proteins in the catalog by approximately five percent (the size of yeast chromosome I is reduced from 102 proteins to 98, yeast chromosome IV from 801 to 763, yeast chromosome IV, VII and XII from 1902 to 1821, E. Coli from 2890 to 2508 and yeast from 6212 to 5908).

FIG. 7 is a plot indicating a number of antibodies that uniquely identifies proteins based on catalog size. From FIG. 7, forty-four antibodies (i.e., epitopes) can differentiate approximately 5908 different proteins of the pruned yeast catalog. FIG. 7 also indicates that the solution size growth can be sub-linear as catalog size increases. Accordingly, between sixty and two-hundred antibodies may be sufficient to distinguish between the approximate sixty thousand human proteins.

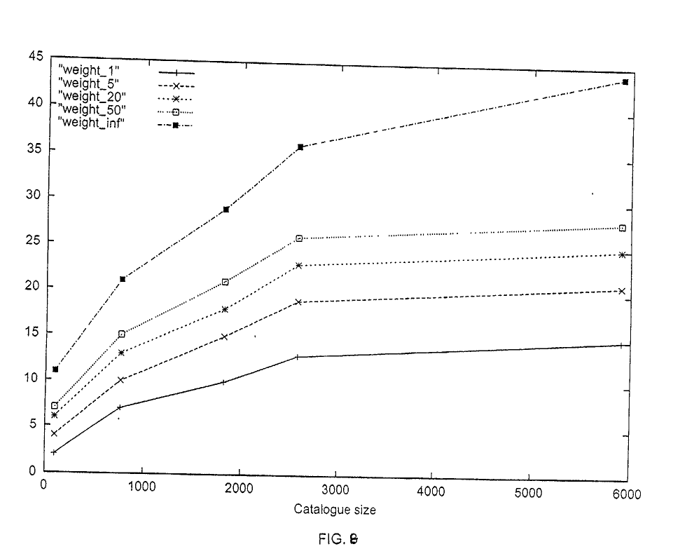

FIG. 8 provides data generated by using molecular mass to differentiate proteins. As FIG. 8 indicates, the methods and systems disclosed herein for identifying a minimum epitope solution set were used when sub-catalogs based on molecular mass (i.e., number of amino acids) were formed. FIG. 8 molecular mass categories included sub-catalogs for proteins that differed by one, five, twenty, fifty, and all (infinite) amino acids. As FIG. 8 indicates, and as shown in FIG. 7, forty-four epitopes can distinguish nearly six thousand proteins when mass is not considered. When a sub-catalog is formed by using molecular mass to identify proteins that differ in size by more than fifty amino acids, and using the disclosed methods and systems to form an epitope solution set, approximately twenty-eight epitopes, or nearly half the amount compared to when molecular mass is not used, can distinguish the proteins. When the exact size of the protein is known, approximately fifteen epitopes/antibodies are necessary to distinguish the proteins, while when the protein is known to within five amino acids, approximately twenty-one epitopes/antibodies may be used. If the protein size can be determined within twenty amino acids, approximately twenty-four epitopes/antibodies can distinguish or identify the proteins.

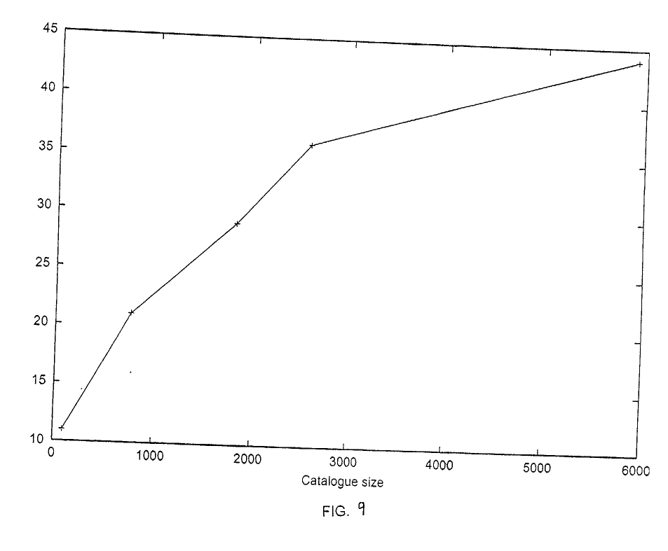

FIG. 9 provides results for differing models of forming antibodies. In a first method, trypsin can be used. In conjunction with the trypsin cleavage, haptens (small molecules) can be added to the amino terminal of a trypsin cleaved fragment and to the carboxyl terminal if the carboxyl terminal molecule is a Lys. It is assumed that antibodies can be built to specifically recognize the haptens and up to three other amino acids. In a second model, specific antibodies can be built against any non-hydrophobic epitope in a trypsin cleaved product. Some amino acids are hydrophobic, and a sequence of hydrophobic amino acids can be in the core of a protein or embedded in a membrane bilayer, making it unlikely that antibodies can be built to recognize them. In a third model, tri-specific antibodies can be built against a non-hydrophobic trimer (three amino acids) occurring in the trypsin cleaved product. In a fourth model, tetra-specific antibodies can be built against a non-hydrophobic tetramer (four amino acids) occurring in a trypsin cleaved product.

Although the plots of FIGS. 7 and 8 utilized the first model, as provided previously herein, FIG. 9 includes the effects of the four aforementioned models. Using a presumption that antibodies can be built against any epitope, FIG. 9 illustrates that thirty-two antibodies can distinguish the yeast protein catalog.

When the trypsin model is used to build antibodies, proteins are cleaved at Lys and Arg residues. Tags can be added to the N-terminal amine of the cleaved fragments. Furthermore, if the protein is cleaved at a Lys, a tag can also be added to the ε-amine of the C-terminal Lys. This is shown schematically below, with the original protein to the left and the trypsin-cleaved products along with the tags, denoted *, to the right.

MLKSA MLK*+*SA

MLRSA MLR+*SA

In an embodiment, it is anticipated that a single antibody can bind to half of the proteins and not to the other half, while the next antibody can bind to half of the proteins that bound to the first and half of those that did not, etc. This can be the case when one could construct a specific antibody that recognizes a subset of the proteins, or when there is a cut cover problem. Using this theory, a minimum number of antibodies to differentiate between a set of n proteins is ┐log₂ n┌, and at least thirteen antibodies are necessary to identify proteins in the yeast proteome; however, this bound can be considered optimistic because it assumes the epitopes can be assigned to the proteins.

FIG. 9 shows that when antibodies are built specific to a non-hydrophobic trimer, the number of antibodies for the yeast catalogue is approximately seventy-three, and when antibodies are built specific to a non-hydrophobic tetramer, the number of antibodies is approximately three-hundred-ninety. It is believed that this size increase is because most tetramers occur in fewer of the proteins and thus will not approximate an optimistic case of each antibody differentiating between half of the proteins.

Table 2 provides solution sets based upon the yeast proteome. The yeast proteome sequences are available at Stanford University's Saccharomyces genome database (SGD, see, Cherry, J. M., Ball, C., Dolinski, K., Dwight, S., Harris, M., Matese, J. C., Sherlock, G., Binkley, G., Jin, H., Weng, S., and Botstein, D. “Saccharomyces Genome Database,” http://genome-www.stanford.edu/Saccharomyces/ (2000); see also, Cherry, J. M., Ball, C., Dolinski, K., Dwight, S., Harris, M., Matese, J. C., Sherlock, G., Binkley, G., Jin, H., Weng, S., and Botstein, D. “Saccharomyces Genome Database,” ftp://genome-ftp.stanford.edu/pub/yeast/SacchDB/ (2000)) Hap1 refers to a fluorescent tag (fluorescein) attached to the ε-amine of the C-terminal lysine of a peptide fragment. Hap2 refers to a fluorescent tag (fluorescein) attached to the N-terminal alpha amine of a peptide fragment.

Referring to Table 1, solutions 1-19, 20, 21-27, 28-43, 44 and 45-56 were independently generated by the same local search heuristic when the candidate epitopes were chosen using the Trypsin model. The nineteen solutions of 1-19 were found when a processor having instructions for implementing the disclosed methods and systems was run for approximately one day.

The Table 1 solutions include the pruned version of the yeast proteome where proteins of length less than one-hundred amino acids or with fewer than five trypsin cleavage sites were removed, and for each pair of closely homologous proteins, one is removed. The resulting catalog was thus 5908 proteins.

Solutions 1-19 were generated by assuming that the candidate epitopes are epitopes of 1-3 amino acids plus the tag, or epitopes of type Lys-Tag2, X₁-Lys-Tag2, X₁-X₂-Lys-Tag2, Tag1-Y₁, Tag1-Y₁-Y₂ or Tag1-Y₁-Y₂-Y₃, where Tag1 and Tag2 are the fluorescent tags (fluorescein), while X₁, X₂, Y₁, Y₂, Y₃ are an amino acid that is consistent with the trypsin cleavage model, which implies that Y₁ cannot be Pro, X₁ and X₂ cannot be Lys or Arg, if Y₁ is Lys or Arg, there cannot be a Y₂ or Y₃, and, if Y₂ is Lys or Arg, there cannot be a Y₃.

Solutions 20-27 are examples of a solution generated by assuming that the candidate epitopes are epitopes of 2-3 amino acids in length, plus the tag, or epitopes of type X₁-Lys-Tag2, X₁-X₂-Lys-Tag2, Tag1-Y₁-Y₂, Tag1-Y₁-Y₂-Y₃, with the same consistency restrictions as before. As with Solutions 1-19, there can be more than one solution of size 42.

Solutions 45-56 were generated under the same model as those for solutions 20-27, but excluding those epitopes that occurred in Solution 20 as candidate epitopes. Even though the size of the epitope solutions set resulting from disallowing many effective epitopes has increased, this increase is not prohibitive.

Solutions 28-43 and 44 exemplify a solution generated by assuming that the candidate epitopes are epitopes of 2-3 amino acids in length, plus the tag, or epitopes of type X-Lys-Tag2, X₁-X₂-Lys-Tag2, Tag1-Y₁-Y₂, Tag1-Y₁-Y₂-Y₃, with the same consistency restrictions as before. In these solutions, amino acids were grouped into meta-classes, thereby reducing the possible candidates to occur from this reduced alphabet of meta-classes of amino-acids. In particular, Solution 28-43 groups both the Leucine and isoleucine aminoacids into one peptide class, thereby reducing the size of the peptide alphabet from 20 to 19, while Solution 44 uses a more extensive grouping (elaborated in the Table description) that reduces the peptide alphabet size from 20 to 14.

The Table 1 solutions reflect a problem of generating a viable solution set of epitopes, where one or more immunogens are not capable of generating a binding reagent suitable for use in the chip. For example, in preparing a chip, binding reagents may be generated to the epitopes listed in any one of the above-described solutions; however, it may be found that one or more epitopes in a selected solution fails to generate a binding reagent capable of binding the epitope with sufficient specificity and/or affinity for the purposes herein. In that case, the solution may be re-run with epitopes that can generate a binding reagent being required in the solution, and with the epitopes that are incapable of generating a binding reagent deleted from the set of identified epitopes. Using this process, a full set of binding reagents can be produced given the advanced state of the art of producing antibodies and recombinant binding reagents by methods such as phage display.

To account for experimental errors, and to create a set of binding reagents that does not vary from protein mixture-to-protein mixture, the aforementioned methods and systems can be modified to consider that the antibody-binding pattern of the protein is non-perfect. Alternately stated, the measured bindings may not necessarily be the same as the bindings calculated by the model. Two distinct types of errors thus exist for computational purposes. The first error includes “false negatives” which includes fragments that are assumed in the computational model to bind but do not bind in practice. The second error includes “false positives” which encompasses fragments that are assumed in the computational model not to bind, but do bind in practice.

Further, depending on the method and system employed to purify the protein to be analyzed, it is likely that more than a single protein will be co-isolated. For instance, if the proteins are isolated by extraction from a two-dimensional gel, two or more proteins may migrate to the same spot in the gel; however, the proteins migrating to the spot may be limited.

The binding reagent (or epitope) set should thus identify most proteins in the presence of errors. One approach to providing a robust solution set includes insisting that all known proteins be distinguished by more than one epitope. If the binding reagent set is to be able to uniquely deconvolve an antibody binding pattern in the presence of a single error, then protein pairs should be differentiated by at least three antibodies. In general, to reconstruct a protein from its antibody binding pattern with k errors, 2k+1 antibodies may be needed to distinguish between each protein pair The number of antibodies needed in such a solution can increase at least roughly linearly with k. To identify two proteins, the antibody-binding pattern of each protein pair can provide a unique signature for that pair. In general, to identify l proteins, the signature of each l-tuple of proteins should be unique. The number of antibodies needed in such a solution can also grow at least linearly with l. A deterministic solution for this problem can be large, depending on the choices of k and l. A correct choice of k and l is not known a priori. Accordingly, a generic chip design may be practicable.

In one embodiment, the inquiry can be directed to antibodies built against epitopes including amino acids close to trypsin cleavage sites. Trypsin cleaves proteins at occurrences of Lys-Xaa and Arg-Xaa, unless Xaa=Pro. Tags can be added to the alpha-amino terminus of the trypsin cleavage products. Binding reagents can be built to recognize an epitope that combines the tag and the two N-terminal amino acids of the trypsin cleavage products. The tag may also be added to the ε-amine of the carboxyl terminal of Lys and binding reagents can be generated to recognize an epitope that combines the tag, the lysine and the two C-terminal amino acids of the trypsin cleavage products adjacent to the lysine residue.

In this example, the generated binding reagent species can be arranged in discrete locations on a chip, specifically a tiled array. The chip containing the binding reagents to molecules of type X₁- . . . Xᵢ-K-T_{C} and T_{A}-Y₁- . . . -Yⱼ, is denoted C(i,j), where T_{A} and T_{C} can be the amino-terminal and carboxyl-terminal tags, respectively, and X₁- and Xᵢ- and Y₁- and Yᵢ can be an amino acid except for K and R, and Y₁ is not P. The chip C(2,2) can therefore include antibodies against epitopes of type X₁-X₂-K-T_{C} and T_{A}-Y₁-Y₂. Table C shows the size of a chip based on a number of antibodies (this number can be easily calculated as 18¹+17*18^{j-1}.

TABLE C
Number of binding reagent species on a given chip

| C(1, 2) | C(2, 2) | C(2, 3) | C(3, 3) |
|---|---|---|---|
| 324 | 630 | 5832 | 11340 |

A statistical scoring model can be constructed to develop a scoring scheme to compare a hypothesis that a protein from a given catalog was bound to the protein chip, with an alternative null hypothesis that the output of the protein chip was random noise. The score can thus signify, for an antibody binding pattern, a likelihood that the binding pattern was not generated by antibodies binding to one of the known proteins, but was random noise. In one embodiment, the score can be a significance score, and in some embodiments, the higher the significance score, the lower the probability that the binding pattern was generated by random noise.

Initially, a mathematical model can be developed and several probability distribution functions can be computed. For example, the probability of occurrence of an epitope can be understood as the likelihood that an epitope occurs at a given antibody binding site, and this likelihood or probability can be expressed as a ratio of the number of binding sites for a given epitope, to the total number of binding sites (for all known epitopes) in the protein catalog. The likelihood that a protein includes an epitope can be based on the number of binding sites in the protein. A raw score can signify the likelihood that a protein is identified, given an antibody binding pattern was computed. A significance score can consider the different sizes of proteins and different number of potential binding sites. In one embodiment, the significance score can be normalized between zero and one. The significance score can be used to accept or reject the null hypothesis.

In computing a statistically based significance score, it was assumed that observed antibody bindings were true bindings of epitopes of proteins or protein fragments in a protein catalog. A true binding can be a binding of an antibody to the epitope to which it was designed to bind. Alternately stated, it was assumed there were not any false positives in the binding data, and hence a binding of an antibody to an epitope that is not a member of p is a binding with some other protein fragment from the catalog. It was also assumed that protein fragments from the catalog occur with the same probability in the experiment as in the catalog. As an example, in the yeast catalog, if an epitope e occurs 100 times in the proteins of yeast and epitope f occurs 10 times, then e is ten times more likely to occur as a positive than f.

A set S of proteins containing a subset P of the epitopes of C(i, j) is given. For reasons discussed below, only a subset P′ of P of the antibody bindings will be identified. A confidence or performance measure in reconstructing S using P′ can be determined from a score that measures the plausibility that protein p contributed to a given pattern P′.

As used herein:

a εb—reads “a in b,” meaning a is part of, or occurs in b.

a ∉b—reads “a is not in b,” meaning a does not occur in b.

eεp—if e is an epitope and p is a protein, this denotes that epitope e occurs in p or that the antibody designed for e will bind to p.

P(a|b)—is the probability that an event a will happen if it is known that event b happens.

A∪B—the union of the two sets, or collections, A and B (this denotes the set or collection of all the members that occur in either A or B, or in both A and B).

∪_{aεb} f(a)—the union of all the events f(a), where a is a member of (or occurs in) b.

Π_{aεb} f(a)—f is some function which is evaluated at points a that occur in b. The Π denotes multiplication and, therefore, f(a)s are multiplied.

Based on the above, an antibody-binding pattern can be modeled as a set, and thus the model can provide a hybridization pattern that can be modeled as a set of 0, 1 random variables e₁, e₂, . . . eₖ, where there is an e for each antibody binding site on a chip, and a measurement P′ of e₁, e₂, . . . eₖ. It is known that P′⊂P=∪_{pεS}∪_{eεp} e for some unknown set of proteins S. The proteins can be provided from a finite catalog, p₁, p₂, . . . , pₙ, and it can be assumed that there is no a priori information available about the distribution of the proteins. Information can be utilized regarding the relative rate of occurrence of the individual epitopes, but the model can be built without postulating other probabilities. Furthermore, the proteins can be ranked according to the plausibility of the proteins belonging to S and a significance score can be determined to reject the hypothesis that P′ is a random sample of protein fragments or random noise.

The number of antibody binding sites in the catalog can be determined to obtain the distribution for the epitopes. The probability that an epitope e occurs at an antibody binding site, μₑ, can thus be expressed as the number of the binding sites in which the epitope occurs relative to the total number of binding sites. This model assumes that a small number of false positives occur, but a large number of false negatives may occur, however other models can differ this assumption by changing the distribution from which the epitopes are drawn, or alternately stated, changing the computation method for μₑ. Other formulations subsequently provided herein are independent of the method by which μₑ is computed.

Accordingly, if p is a protein with one antibody binding site, a model can assume that this binding site is e with probability μₑ. Such a model also assumes that the probability that a protein contains e depends on the number of binding sites in p. If it is assumed that the epitopes occur independently in a protein, the probability that an epitope e occurs in p, given p has b binding sites, is:

P(eεp):=1−(1−μₑ)^{b}

According to the above formulation, the probability that an epitope e occurs in protein p is one minus the probability that an epitope e does not occur in any of the b binding sites in protein p. Because the chance of appearance of an epitope at a binding site is assumed to be independent of the same on another site, this latter probability can be expressed as the product of the chance of the epitope e not occurring in the site (1−μₑ) raised to the power of b (i.e., probability of co-occurrence of a pair of independent events is the product of their occurrence probabilities).

Because it is assumed that the occurrence of one epitope does not depend upon the occurrence of other epitopes, the probability space is not the set of all known proteins that have b binding sites, but the set of proteins generated by a series of binomial generators. The expected number of epitopes generated by this generator is approximately the same as if it were required that the protein has b binding sites. The generator may, with low probability, generate less than b epitopes.

Given a protein p and the results of a hybridization experiment P′, a score for protein p can be expressed as

$${Π}_{e ∈ {P}^{′} ⋂ p} \frac{1}{P ( e ∈ p )} ,$$

where P(eεp) is defined above. In an instance of two epitopes, assuming independence, the probability that p contains two epitopes is the product of the probabilities that p contains each epitope. The score weighs each positively identified epitope with a weight equal to the inverse of the probability that the epitope occurred in p, assuming the probability model.

A significance score can be computed by scaling the score by the expected score for a protein, given the number of binding sites in p and given the signal P′. Again assuming that the probability of each of the epitopes of P′ occurring in p is independent of the other epitopes of P′ occurring in p, the significance score can be expressed as:

$${E}_{p} ( {{Π}_{e ∈ {P}^{′}} ( \frac{1}{P ( e ∈ p )} )}^{e ∈ p} ) = {Π}_{e ∈ {P}^{′}} {{E}_{p} ( \frac{1}{P ( e ∈ p )} )}^{e ∈ p} = {Π}_{e ∈ {P}^{′}} ( 1 \cdot ( 1 - P ( e ∈ p ) ) + \frac{1}{P ( e ∈ p )} \cdot P ( e ∈ p ) ) = {Π}_{e ∈ {P}^{′}} ( 2 - P ( e ∈ p ) )$$

Computing the expectation over |P′| binomial generators, each generating an epitope with probability P(eεp), and assuming the expected number of binding sites generated by the generator is b, the above formulation for significance score can be expressed as:

$$\frac{{Π}_{e ∈ {P}^{′} ⋂ p} \frac{1}{P ( e ∈ p )}}{{Π}_{e ∈ {P}^{′}} ( 2 - P ( e ∈ p ) )}$$

The significance score can thus be utilized in evaluating the null hypothesis that the antibody binding pattern occurred by chance (i.e., random noise). The expectation of the significance score is one because the significance score is a random variable divided by the expectation of the same random variable. Using Markov's inequality, if a protein p has a significance score x then no more than a 1/x fraction of the randomly generated proteins of length b have a higher significance score than p. Using only this bound, if the highest significance score in the catalog is approximately the size of the catalog, then the conjecture that P′ is random noise cannot be rejected. It is therefore preferable that the significance score be considerably larger than the catalog size. Perkins et al. (Perkins, D. N., Pappin, D. J. C., Creasy, D. M., Cottrell, J. S. (1999), “Probability-Based Protein Identification By Searching Databases Using Mass Spectometry Data,” Electrophoresis 20, 3551-3567) suggest a significance score that is twenty times greater than the catalog size.

Those with ordinary skill in the art will recognize that Markov's inequality is not only inequality that can be used to provide a bound, and other inequalities may similarly be used.

The scoring scheme does not consider orthogonal information obtained as a byproduct of experimentation or the method used for isolating the protein. Orthologous information about the protein, such as its size, charge or type, can reduce the catalog size and decrease the number of matches needed to obtain a significant match. Accordingly, such orthogonal information can strengthen the scoring scheme.

Because the denominator of the significance score can increase at approximately 2^{|P′|}, false positives can lower the significance score by nearly a factor of two. False negatives can also affect the significance score. If p has more antibody binding sites than q, then the probability that an epitope occurs in p increases, lowering the score of a match. Furthermore, as the total number of matches decreases, the significance score decreases.

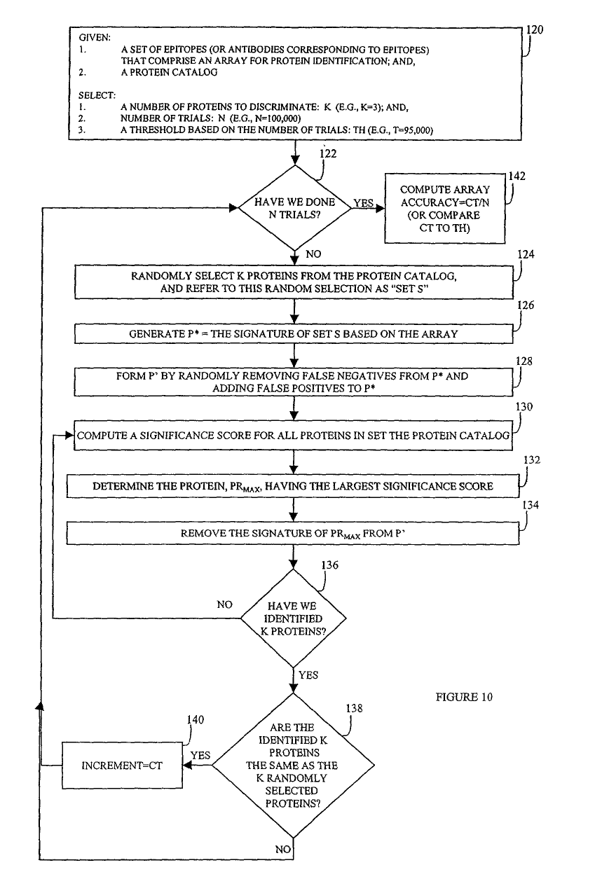

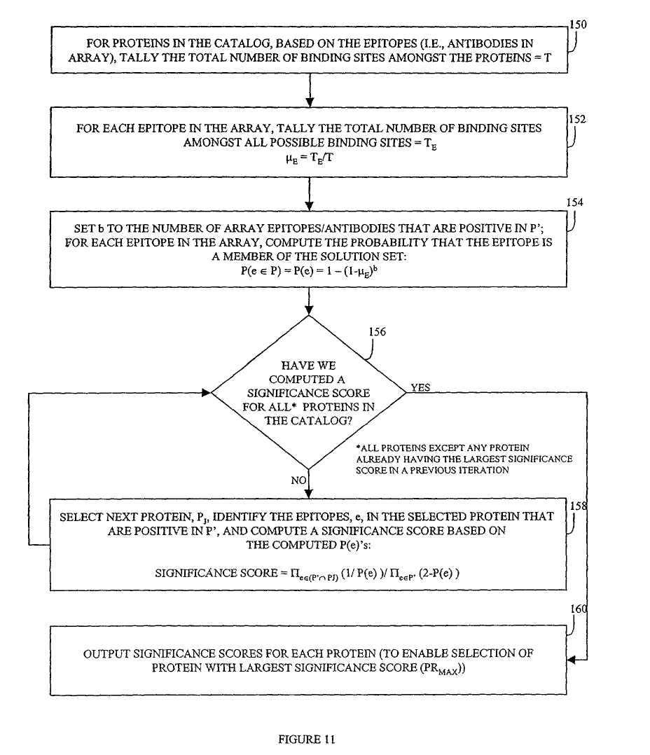

FIGS. 10 and 11 provide varying detail for one embodiment of the statistical scoring model provided herein for evaluating the performance of a given epitope/antibody/binding reagent array to identify proteins from a protein catalog. As FIG. 10 indicates, given a protein catalog and partial solution set of epitopes 120 (wherein such partial solution set can be provided using the methods and systems provided herein with reference to FIGS. 2 and 3, etc.), the FIG. 10 illustrated methods and systems include determining a number of proteins, k, to discriminate using the partial solution set, a number of trials, N, and a threshold, TH, that can be based on the number of trials and the desired performance. In the illustrated system, k is selected to be three, N is selected to be 100,000, and for a 95% success rate, TH is selected to be 95,000. Those with ordinary skill in the art will recognize that the disclosed methods and systems are not limited to the aforementioned values, and other values can be selected based on the application for the scoring model. For example, k is not limited, but for some assumptions provided herein, k can preferably be between one and five. In other embodiments, the number of trials was 200,000. Accordingly, as in the other illustrations provided herein, the examples of FIG. 10 are for illustration and not limitation.

Referring again to FIG. 10, N trials can be performed 122, wherein a trial can include randomly selecting a set “S” of k proteins from the protein catalog 124, generating an array signature, P*, based on the epitopes of the array and the epitopes in the k randomly chosen proteins in S 126, and utilizing a probability distribution model for false negatives and a probability distribution model for false positives to augment the array signature, P*, to form P′ 128. As previously provided herein, and as shall be provided in additional and optional detail in FIG. 11, a significance score can be computed for the proteins in the protein catalog using P′ 130. The protein having the greatest significance score, designated PRmax, can be determined 132 and the protein signature for PRmax can be removed from P′ 134. PRmax thus can be understood to be an identified protein of protein set S. If k proteins have not yet been identified 136, the process continues at 130 and as shown in FIG. 10, until k proteins are identified. Once k proteins are identified 136, the identified k proteins can be compared to set S. If the identified k proteins are identical to set S 138, then the FIG. 10 embodiment deems this trial a success for the protein identification trial, and a counter, CT, can be updated 140. This process can continue at 122 until N trials are performed. Once N trials are performed 122, the overall success rate for the protein array/chip/epitope group can be computed through a percentage of success, or CT/N. Alternately, CT can be compared to TH to determine whether the desired performance measure was achieved.

FIG. 11 provides additional and optional features for computing a significance score for a method and system according to FIG. 10, although the FIG. 11 embodiment is not limited to the systems and methods of FIG. 10. As previously provided, for methods and systems according to FIG. 10, the significance score can be computed at 130. Referring to FIG. 11, in computing the significance score, a value T can be computed that represents the total binding sites for epitopes in the protein catalog 150. In an embodiment, the number of binding sites for the epitopes can be individually computed, where such number can be represented by T_{E}, and the T_{E} for the epitopes can be summed to provide a value for T. Accordingly, a value μ_{E} can be computed for an epitope, where μ_{E} can represent the ratio of binding sites for the epitope in the catalog to the total number of binding sites in the protein catalog 152.

As 154 indicates, a value b can be computed that represents the number of array sites or epitopes in signature P′ that are positive. For an embodiment according to FIG. 10, P′ can represent the actual signature for the randomly selected k proteins of set S, where such actual signature is thereafter altered using probabilistic models to add false negatives and false positives. Returning again to 154 of FIG. 11, and as previously provided herein, a probability that a selected epitope is part of the solution can be computed as P(e)=1−(1−μ_{E})^{b}. A significance score can thus be computed for the proteins in the protein catalog 156 using the computations from 150, 152, and 154.

As provided previously herein, for a selected protein in the protein catalog, a significance score can be computed based on the epitopes in the selected protein and the associated probabilities that those epitopes are part of P′ 158. This significance score, previously provided herein, can be expressed as:

$$\frac{{Π}_{e ∈ {P}^{′} ⋂ p} \frac{1}{P ( e ∈ p )}}{{Π}_{e ∈ {P}^{′}} ( 2 - P ( e ∈ p ) )}$$

Once significance scores are computed 156 for the proteins, the maximum significance score can be determined 160.

The aforementioned statistical model was validated empirically for a case when S includes a single protein, and when S contains multiple proteins. The experiments were performed for a C(2, 2) chip and yeast catalogue pruned for close homologues and proteins that either contain fewer than 100 amino acids or fewer than 5 trypsin cleavage sites. Simple computational experiments were performed on the C(2, 2) chip. One control protein was randomly chosen from the pruned catalogue of yeast proteins and it was determined which protein had the highest significance score, as described above. This experiment was repeated 2000 times and it was determined how often the highest scoring protein was the control protein. The experiment was repeated for values 0; 1; 2 and 5 percent rates of false positive error and for 0; 20 and 40 percent rates of false negative error. The results are shown in Table D.

Table D shows the percentage of times a randomly generated protein using the scoring function was correctly identified. With false negative rates 0, 20 and 40 and false positive rates 0, 1, 2, and 5, data are averaged over 2000 trials. As Table D indicates, when the false positive rate was set to at most 2%, the highest scoring protein was the control protein in more than 99% of the trials, even if the false negative rate was set to 40%. In the worst case examined, when the false positive rate was set at 5% and the false negative rate was set at 40%, the method failed to identify the control protein as the highest scoring protein in roughly 5% of the trials. Note that when the false negatives and false positives were set to 0%, the control protein was not identified in about 0.1% of the trials, as two (non-homologous) proteins may have the same antibody-binding pattern.

TABLE D

| % False | % False Positive | | | |
|---|---|---|---|---|
| Negative | 0 | 1 | 1 | 5 |
| 0 | 99.9 | 99.8 | 99.8 | 99.25 |
| 20 | 99.95 | 99.9 | 99.75 | 97.6 |
| 40 | 99.85 | 99.85 | 99.2 | 94.35 |

One algorithm for identifying multiple proteins includes determining the highest scoring protein, removing the binding pattern of that protein, and repeating the algorithm on the remaining problem. Table E illustrates the percentage of runs (averaged over 2000 trials) the algorithm correctly identified all k proteins from a noisy binding pattern of k proteins, where k ranges from 1 through 5, the false negative rate was 0% and the false positive rate was 0, 1, 2 and 5%. Table F provides a false negative rate of 20%, while Table G provides a false negative rate of 40%.

Table E indicates that when there are no false positives and no false negatives, proteins were correctly identified in over 99% of experiments if |S| 4, and 97.95% experiments if |S|=5. Tables E, F and G indicate that the number of correct identifications decreases with the number of false positives, the number of false negatives, and the number of proteins in the protein sample to be identified. In one embodiment, the false positive rate can be preferably maintained at less than 5%, unless the protein samples include one or two proteins, or if some experimental error is acceptable.

TABLE E
0% False Negatives

| | False Positives | | | |
|---|---|---|---|---|
| No. Proteins | 0 | 1 | 2 | 5 |
| 1 | 99.9 | 99.8 | 99.8 | 99.25 |
| 2 | 99.95 | 99.8 | 99.5 | 96.6 |
| 3 | 99.65 | 99.2 | 98.5 | 93.15 |
| 4 | 99.25 | 97.65 | 96.1 | 85.1 |
| 5 | 97.95 | 95.4 | 91.45 | 75.55 |

TABLE F
20% False Negatives

| | False Positives | | | |
|---|---|---|---|---|
| No. Proteins | 0 | 1 | 2 | 5 |
| 1 | 99.95 | 99.9 | 99.75 | 97.6 |
| 2 | 99.75 | 99.25 | 98.45 | 92.15 |
| 3 | 99.6 | 98.25 | 95.75 | 85.4 |
| 4 | 98.6 | 95.05 | 91.35 | 73.1 |
| 5 | 91.7 | 88.7 | 82.6 | 54.4 |

TABLE G
40% False Negatives

| | False Positives | | | |
|---|---|---|---|---|
| No. Proteins | 0 | 1 | 2 | 5 |
| 1 | 99.85 | 99.85 | 99.2 | 94.35 |
| 2 | 99.35 | 97.0 | 93.45 | 81.85 |
| 3 | 97.95 | 93.45 | 88.5 | 67.15 |
| 4 | 97.2 | 86.1 | 80.3 | 49.7 |
| 5 | 92.6 | 78.65 | 65.65 | 24.8 |

Generally, as the number of false negatives increases, the number of false positives decreases, the number of proteins decreases, and the predication accuracy decreases.

The significance score can extend to scoring multiple proteins and the significance score for a set of proteins, p₁, p₂, . . . , pₖ can be expressed as:

$$\frac{{Π}_{e ∈ {P}^{′} ⋂ ( ⋃ \begin{matrix}k \\ i = 1\end{matrix} {p}_{i} )} \frac{1}{P ( e ∈ ⋃ \begin{matrix}k \\ i = 1\end{matrix} {p}_{i} )}}{{Π}_{e ∈ {P}^{′}} ( 2 - P ( e ∈ ⋃ \begin{matrix}k \\ i = 1\end{matrix} p ) )}$$

Note that the number of k-tuples in a catalogue of size n is

$$( \begin{matrix}n \\ k\end{matrix} ) .$$

For the score to be significant it should be considerably larger than

$$( \begin{matrix}n \\ k\end{matrix} ) .$$

It is well known that the highest scoring set of proteins (using the aforementioned score for sets) may not be the ones found by the greedy algorithm presented above. Because of the time complexities involved it is not efficient to search over all k-tuples. An extension of the method combines this score with a heuristic to compute the most significant score of sets of proteins.

One disadvantage of the statistical model is the probability distribution over which the expectation is calculated. It is possible that when |P′| is significantly larger than |p|, that the calculated expectation can be greater than the value of the maximal significance score obtained for a protein, because expectation is computed for proteins that are randomly generated by a generator that generates in expectation |p| binding sites, rather than calculating the expectation over proteins with |p| binding sites. As the significance score weighs multiple bindings heavily, proteins generated by the generator that have more than |p| bindings sites can increase the value in the expectation calculation. A solution can include redefining the probability distribution.

In combining the methods and systems of FIGS. 2-3 with the methods and systems of FIGS. 10-11, a minimization problem can be provided with an objective to minimize the number of epitopes used in a chip while recognizing single proteins with high confidence. The minimization problem can be expressed as:

$${Min}_{x} {\sum }_{e ∈ A} {x}_{e}$$

$${{Π}_{e ∈ p} ( \frac{1}{( 2 - P ( e ∈ p ) ) P ( e ∈ p )} )}^{{x}_{e}} \geq C for proteins p$$

$${x}_{e} ∈ \{ 0 , 1 \}$$

In this formulation, A is the set of antibodies, and the number of second inequality (constraints) included is one per protein in the catalog. The variable xₑ is a binary decision variable denoting whether the epitope e should be included in the chip. C is a constant specifying the desired confidence in the identification of a given protein (e.g. 20 times catalogue size). Accordingly, the above formulation minimizes the number of antibodies subject to a constraint that if the epitopes of a protein p in the binding pattern are seen, then there is a significant score at a level (specified by C) for p using this chip. The optimization problem is a non-linear integer program that can be linearized by taking logarithms of the constraints, thereby reducing the above formulation to the following minimization problem:

$${Min}_{x} {\sum }_{e ∈ A} {x}_{e}$$

$${\sum }_{e ∈ p} log ( \frac{1}{( 2 - P ( e ∈ p ) ) P ( e ∈ p )} ) {x}_{e} \geq C for proteins p$$

$${x}_{e} ∈ \{ 0 , 1 \}$$

The above expression is a linear integer program with n constraints.

The methods and systems described herein are not limited to a particular hardware or software configuration, and may find applicability in many computing or processing environments. The methods and systems can be implemented in hardware or software, or a combination of hardware and software. The methods and systems can be implemented in one or more computer programs, where a computer program can be understood to include a group of processor executable instructions. The computer program(s) can execute on one or more programmable processors, and can be stored on one or more storage medium readable by the processor (including volatile and non-volatile memory and/or storage elements), one or more input devices, and/or one or more output devices. The processor thus can access one or more input devices to obtain input data, and can access one or more output devices to communicate output data. The input and/or output devices can include one or more of the following: Random Access Memory (RAM), Redundant Array of Independent Disks (RAID), floppy drive, CD, DVD, magnetic disk, internal hard drive, external hard drive, memory stick, or other storage device capable of being accessed by the processor as provided herein, where such aforementioned examples are not exhaustive, and are for illustration and not limitation.

The computer program(s) is preferably implemented using one or more high level procedural or object-oriented programming languages to communicate with a computer system; however, the program(s) can be implemented in assembly or machine language, if desired. The language can be compiled or interpreted.

The processor(s) can thus be embedded in one or more devices that can be operated independently or together in a networked environment, where the network can include, for example, a Local Area Network (LAN), wide area network (WAN), and/or can include an intranet and/or the internet and/or another network. The network(s) can be wired or wireless or a combination thereof and can use one or more communications protocols to facilitate communications between the different processors. The processors can be configured for distributed processing and can utilize, in some embodiments, a client-server model as needed.

The device(s) that includes the processor(s) can thus include a personal computer(s), workstation (e.g., Sun, HP), personal digital assistant (PDA), handheld device such as cellular telephone, or another device capable of being integrated with a processor(s) that can operate as provided herein. Accordingly, the devices provided herein are not exhaustive and are provided for illustration and not limitation.

Although the methods and systems have been described relative to a specific embodiment thereof, they are not so limited. Obviously many modifications and variations may become apparent in light of the above teachings. For example, although the methods and systems discussed illustrations using a yeast proteome to provide a protein catalog, a protein catalog as provided herein can include two or more proteins for which a distinguishing or identifying array is desired to be designed. As provided previously herein, certain values for numbers of trials can be based on the desired application and/or performance, and thus are not limited to the illustrative values provided herein. Although the illustrated systems and methods discussed partial solution sets which could be further refined, in some embodiments, such refining may not be performed, and thus such partial solution sets can be understood to be a solution set.

Many additional changes in the details, materials, and arrangement of parts, herein described and illustrated, can be made by those skilled in the art. Accordingly, it will be understood that the following claims are not to be limited to the embodiments disclosed herein, can include practices otherwise than specifically described, and are to be interpreted as broadly as allowed under the law.

TABLE 2

| | | | Number of |
|---|---|---|---|
| Solution | | | epitopes in |
| No. | Criteria | Solution | proteome |
| 1 | 38 epitopes; | Met-Lys-Tag2 | 2741 |
| | 1-3 amino acids plus | Tyr-Lys-Tag2 | 3314 |
| | the tag | Pro-Lys-Tag2 | 3694 |
| | | Tag1-His- | 3414 |
| | | Tag1-Asn- | 4956 |
| | | Tag1-Leu-Leu- | 2161 |
| | | Tag1-Glu-Ile- | 1285 |
| | | Tag1-Leu-Ser- | 1823 |
| | | Tag1-Asp-Leu- | 1422 |
| | | Phe-Lys-Tag2 | 3782 |
| | | Glu-Lys-Tag2 | 4496 |
| | | Tag1-Cys- | 2440 |
| | | Leu-Lys-Tag2 | 4976 |
| | | Tag1-Ser-Ile- | 1370 |
| | | Tag1-Gly-Ser- | 953 |
| | | Trp-Lys-Tag2 | 1788 |
| | | Gly-Lys-Tag2 | 4167 |
| | | Val-Lys-Tag2 | 4249 |
| | | Asp-Asn-Lys-Tag2 | 531 |
| | | Ser-Ser-Lys-Tag2 | 1363 |
| | | Leu-Val-Lys-Tag2 | 931 |
| | | Tag1-Ser-Leu- | 1910 |
| | | Tag1-Phe- | 4606 |
| | | Tag1-Gln- | 4405 |
| | | Tag1-Ala-Ala- | 909 |
| | | Tag1-Ser-Glu- | 955 |
| | | Tag1-Met- | 3044 |
| | | Tag1-Ile-Leu- | 1691 |
| | | Tag1-Thr- | 4948 |
| | | Tag1-Glu-Asn- | 1090 |
| | | Tag1-Asp-Ser- | 1086 |
| | | Tag1-Ile- | 5113 |
| | | Ile-Ile-Lys-Tag2 | 748 |
| | | Tag1-Tyr- | 4329 |
| | | Tag1-Tyr-Ala-Arg- | 33 |
| | | Ile-Val-Lys-Tag2 | 642 |
| | | Tag1-Asn-Phe-Leu- | 91 |
| | | Asn-Gln-Lys-Tag2 | 286 |
| 2 | 38 epitopes | Met-Lys-Tag2 | 2741 |
| | 1-3 amino acids plus | Tag1-Ile-Leu- | 1691 |
| | the tag | Tyr-Lys-Tag2 | 3314 |
| | | Tag1-Leu-Gly- | 1085 |
| | | Tag1-Ala- | 4770 |
| | | Tag1-Trp- | 2258 |
| | | Thr-Lys-Tag2 | 4245 |
| | | Tag1-Ser-Leu- | 1910 |
| | | Tag1-His- | 3414 |
| | | Tag1-Ser-Glu- | 955 |
| | | Tag1-Ala-Ala- | 909 |
| | | Val-Lys-Tag2 | 4249 |
| | | Tag1-Cys- | 2440 |
| | | Tag1-Asp-Ile- | 1165 |
| | | Tag1-Asn- | 4956 |
| | | Tag1-Glu-Asn- | 1090 |
| | | Tag1-Ile-Ser- | 1322 |
| | | Tag1-Asn-Leu- | 1429 |
| | | Tag1-Asn-Ser- | 1287 |
| | | Tag1-Gln- | 4405 |
| | | Ser-Lys-Tag2 | 4837 |
| | | Tag1-Thr-Asn- | 794 |
| | | Tag1-Asp-Ser- | 1086 |
| | | Ile-Val-Lys-Tag2 | 642 |
| | | Phe-Lys-Tag2 | 3782 |
| | | Gly-Lys-Tag2 | 4167 |
| | | Pro-Lys-Tag2 | 3694 |
| | | Tag1-Gly- | 4629 |
| | | Gln-Lys-Tag2 | 3533 |
| | | Tag1-Leu-Ser- | 1823 |
| | | Leu-Lys-Tag2 | 4976 |
| | | Trp-Lys-Tag2 | 1788 |
| | | Tag1-Glu-Ile- | 1285 |
| | | Asp-Asn-Lys-Tag2 | 531 |
| | | Tag1-Thr-Gly- | 792 |
| | | Asp-Asp-Lys-Tag2 | 565 |
| | | Tag1-Asn-Phe-Leu- | 91 |
| | | Asn-Gln-Lys-Tag2 | 286 |
| 3 | 38 epitopes | Phe-Lys-Tag2 | 3782 |
| | 1-3 amino acids plus | Tag1-Ser-Leu- | 1910 |
| | the tag | Tag1-Gln- | 4405 |
| | | Tag1-Ser-Ser- | 1768 |
| | | Trp-Lys-Tag2 | 1788 |
| | | Tag1-Gly-Ser- | 953 |
| | | Tag1-Thr-Leu- | 1459 |
| | | Tyr-Lys-Tag2 | 3314 |
| | | Thr-Lys-Tag2 | 4245 |
| | | Tag1-Trp- | 2258 |
| | | Tag1-Glu-Glu- | 1322 |
| | | Met-Lys-Tag2 | 2741 |
| | | Asn-Lys-Tag2 | 4207 |
| | | Tag1-Leu-Leu- | 2161 |
| | | Leu-Leu-Lys-Tag2 | 1611 |
| | | Tag1-Ile- | 5113 |
| | | Tag1-Ile-Leu- | 1691 |
| | | Tag1-Leu-Ser- | 1823 |
| | | Val-Lys-Tag2 | 4249 |
| | | Tag1-Ala- | 4770 |
| | | Tag1-Asn-Leu- | 1429 |
| | | Tag1-Asp-Gly- | 800 |
| | | Tag1-Glu-Asn- | 1090 |
| | | Leu-Lys-Tag2 | 4976 |
| | | Tag1-Asn-Val- | 954 |
| | | Ala-Lys-Tag2 | 4233 |
| | | Asp-Asn-Lys-Tag2 | 531 |
| | | Pro-Lys-Tag2 | 3694 |
| | | Tag1-Cys- | 2440 |
| | | Tag1-Ser-Ile- | 1370 |
| | | Gly-Lys-Tag2 | 4167 |
| | | Tag1-Asp- | 4856 |
| | | Tag1-Tyr-Ala- | 517 |
| | | Tag1-Asp-Thr- | 761 |
| | | Tag1-Val-Pro-Leu- | 102 |
| | | Ile-Val-Lys-Tag2 | 642 |
| | | Tag1-Asn-Phe-Leu- | 91 |
| | | Asn-Gln-Lys-Tag2 | 286 |
| 4 | 38 epitopes | Leu-Lys-Tag2 | 4976 |
| | 1-3 amino acids plus | Val-Lys-Tag2 | 4249 |
| | the tag | Pro-Lys-Tag2 | 3694 |
| | | Tag1-Cys- | 2440 |
| | | Tag1-Gly- | 4629 |
| | | Tag1-Ser-Ser- | 1768 |
| | | Tyr-Lys-Tag2 | 3314 |
| | | Tag1-Ile-Leu- | 1691 |
| | | Met-Lys-Tag2 | 2741 |
| | | Tag1-Glu-Asn- | 1090 |
| | | Phe-Lys-Tag2 | 3782 |
| | | Tag1-Gln- | 4405 |
| | | Tag1-Asp- | 4856 |
| | | Tag1-Asn-Leu- | 1429 |
| | | His-Lys-Tag2 | 2388 |
| | | Tag1-Tyr- | 4329 |
| | | Tag1-Gly-Ser- | 953 |
| | | Tag1-Val-Val- | 934 |
| | | Trp-Lys-Tag2 | 1788 |
| | | Tag1-His- | 3414 |
| | | Thr-Lys-Tag2 | 4245 |
| | | Tag1-Met- | 3044 |
| | | Tag1-Ser-Glu- | 955 |
| | | Tag1-Asp-Ser- | 1086 |
| | | Tag1-Glu-Ile- | 1285 |
| | | Tag1-Ser-Leu- | 1910 |
| | | Cys-Lys-Tag2 | 1607 |
| | | Gln-Lys-Tag2 | 3533 |
| | | Tag1-Ala-Val- | 833 |
| | | Tag1-Ser- | 5298 |
| | | Ser-Ser-Lys-Tag2 | 1363 |
| | | Asp-Asn-Lys-Tag2 | 531 |
| | | Tag1-Thr- | 4948 |
| | | Asp-Gly-Lys-Tag2 | 613 |
| | | Tag1-Ser-Ala-Ala- | 77 |
| | | Asp-Asp-Lys-Tag2 | 565 |
| | | Ile-Val-Lys-Tag2 | 642 |
| | | Tag1-Asn-Phe-Leu- | 91 |
| 5 | 38 epitopes | Ala-Lys-Tag2 | 4233 |
| | 1-3 amino acids plus | Tag1-Ile-Leu- | 1691 |
| | the tag | Leu-Lys-Tag2 | 4976 |
| | | Tag1-Gly- | 4629 |
| | | Leu-Leu-Lys-Tag2 | 1611 |
| | | Tag1-Glu-Leu- | 1765 |
| | | Thr-Lys-Tag2 | 4245 |
| | | Tag1-Met- | 3044 |
| | | Ile-Lys-Tag2 | 4370 |
| | | Tag1-Cys- | 2440 |
| | | Tag1-Glu-Asn- | 1090 |
| | | Met-Lys-Tag2 | 2741 |
| | | Tag1-Ser-Glu- | 955 |
| | | Tag1-Gln- | 4405 |
| | | Tag1-Ser-Leu- | 1910 |
| | | Tag1-Asn- | 4956 |
| | | Tag1-Asp-Leu- | 1422 |
| | | Tag1-Glu-Ile- | 1285 |
| | | Tag1-Gly-Ser- | 953 |
| | | Tag1-Ser-Val- | 1125 |
| | | Tag1-Asn-Leu- | 1429 |
| | | Tag1-Ala- | 4770 |
| | | Tag1-Tyr- | 4329 |
| | | Tyr-Lys-Tag2 | 3314 |
| | | Asp-Asp-Lys-Tag2 | 565 |
| | | Tag1-Asp-Ser- | 1086 |
| | | Pro-Lys-Tag2 | 3694 |
| | | Tag1-Leu-Ser- | 1823 |
| | | Asn-Lys-Tag2 | 4207 |
| | | Tag1-Thr-Leu- | 1459 |
| | | Tag1-Ser-Ser- | 1768 |
| | | Tag1-Leu-Leu- | 2161 |
| | | Tag1-Thr-Ser- | 1127 |
| | | Tag1-Ala-Ala- | 909 |
| | | Asp-Tyr-Lys-Tag2 | 374 |
| | | Tag1-Ser-Leu-Tyr- | 98 |
| | | Tag1-Asn-Phe-Leu- | 91 |
| | | Asn-Gln-Lys-Tag2 | 286 |
| 6 | 38 epitopes | Pro-Lys-Tag2 | 3694 |
| | 1-3 amino acids plus | Met-Lys-Tag2 | 2741 |
| | the tag | Tyr-Lys-Tag2 | 3314 |
| | | Tag1-Asn-Leu- | 1429 |
| | | Trp-Lys-Tag2 | 1788 |
| | | Tag1-Gly-Ser- | 953 |
| | | Tag1-His- | 3414 |
| | | Thr-Lys-Tag2 | 4245 |
| | | Phe-Lys-Tag2 | 3782 |
| | | Tag1-Leu-Leu- | 2161 |
| | | Tag1-Glu- | 4977 |
| | | Tag1-Gly- | 4629 |
| | | Tag1-Glu-Asn- | 1090 |
| | | Tag1-Ile-Leu- | 1691 |
| | | Tag1-Asn- | 4956 |
| | | Tag1-Leu-Ser- | 1823 |
| | | Ile-Lys-Tag2 | 4370 |
| | | Tag1-Val-Ile- | 1011 |
| | | Leu-Leu-Lys-Tag2 | 1611 |
| | | Asn-Lys-Tag2 | 4207 |
| | | Tag1-Asp-Leu- | 1422 |
| | | Tag1-Cys- | 2440 |
| | | Tag1-Asp-Ser- | 1086 |
| | | Tag1-Phe- | 4606 |
| | | Tag1-Ser-Leu- | 1910 |
| | | Asp-Asp-Lys-Tag2 | 565 |
| | | Tag1-Ala-Gly- | 613 |
| | | Ala-Lys-Tag2 | 4233 |
| | | Tag1-Thr-Ser- | 1127 |
| | | Tag1-Leu-Ala- | 1302 |
| | | Leu-Lys-Tag2 | 4976 |
| | | Tag1-Glu-Ile- | 1285 |
| | | Asp-Lys-Tag2 | 3940 |
| | | Tag1-Ser-His- | 529 |
| | | Tag1-Tyr-Ile-Glu- | 63 |
| | | Tag1-Ala- | 4770 |
| | | Tag1-Asn-Phe-Leu- | 91 |
| | | Asn-Gln-Lys-Tag2 | 286 |
| 7 | 38 epitopes | Tag1-Met- | 3044 |
| | 1-3 amino acids plus | Tag1-His- | 3414 |
| | the tag | Cys-Lys-Tag2 | 1607 |
| | | Met-Lys-Tag2 | 2741 |
| | | Asp-Lys-Tag2 | 3940 |
| | | Tag1-Ser-Asn- | 1138 |
| | | Tag1-Asn-Leu- | 1429 |
| | | Ser-Lys-Tag2 | 4837 |
| | | Tag1-Cys- | 2440 |
| | | Thr-Lys-Tag2 | 4245 |
| | | Tag1-Gly-Ser- | 953 |
| | | Tag1-Glu-Val- | 972 |
| | | Tag1-Leu-Ser- | 1823 |
| | | His-Lys-Tag2 | 2388 |
| | | Phe-Lys-Tag2 | 3782 |
| | | Tag1-Ala-Val- | 833 |
| | | Tag1-Phe- | 4606 |
| | | Tyr-Lys-Tag2 | 3314 |
| | | Tag1-Glu-Asn- | 1090 |
| | | Val-Lys-Tag2 | 4249 |
| | | Tag1-Ser-Glu- | 955 |
| | | Asp-Asn-Lys-Tag2 | 531 |
| | | Tag1-Ile-Leu- | 1691 |
| | | Leu-Lys-Tag2 | 4976 |
| | | Pro-Lys-Tag2 | 3694 |
| | | Ile-Lys-Tag2 | 4370 |
| | | Tag1-Ser-Leu- | 1910 |
| | | Tag1-Val-Leu- | 1391 |
| | | Tag1-Ser-Thr- | 1249 |
| | | Tag1-Ile- | 5113 |
| | | Tag1-Ser-Ser- | 1768 |
| | | Tag1-Thr- | 4948 |
| | | Tag1-Leu-Pro- | 1021 |
| | | Tag1-Asn-Val- | 954 |
| | | Tag1-Ile-His- | 412 |
| | | Tag1-Ser-Leu-His- | 84 |
| | | Ile-Val-Lys-Tag2 | 642 |
| | | Tag1-Asn-Phe-Leu- | 91 |
| 8 | 38 epitopes | Met-Lys-Tag2 | 2741 |
| | 1-3 amino acids plus | Tag1-Thr-Leu- | 1459 |
| | the tag | Tag1-Gly- | 4629 |
| | | Tag1-Gln- | 4405 |
| | | Asp-Lys-Tag2 | 3940 |
| | | Tag1-Cys- | 2440 |
| | | Tag1-Met- | 3044 |
| | | Tag1-Asn-Leu- | 1429 |
| | | Leu-Lys-Tag2 | 4976 |
| | | Tag1-Glu-Val- | 972 |
| | | Tag1-Asp-Asp- | 921 |
| | | Val-Lys-Tag2 | 4249 |
| | | Tag1-Ala- | 4770 |
| | | Tyr-Lys-Tag2 | 3314 |
| | | Trp-Lys-Tag2 | 1788 |
| | | Tag1-Trp- | 2258 |
| | | Ser-Ser-Lys-Tag2 | 1363 |
| | | Tag1-Tyr-Val- | 655 |
| | | Tag1-Ser-Leu- | 1910 |
| | | Ile-Lys-Tag2 | 4370 |
| | | Tag1-Glu-Asn- | 1090 |
| | | Tag1-Ser- | 5298 |
| | | Tag1-Tyr-Phe- | 577 |
| | | Tag1-Ser-Glu- | 955 |
| | | Pro-Lys-Tag2 | 3694 |
| | | Thr-Lys-Tag2 | 4245 |
| | | Tag1-Ile-Leu- | 1691 |
| | | Tag1-Leu-Leu- | 2161 |
| | | Tag1-His- | 3414 |
| | | Asp-Tyr-Lys-Tag2 | 374 |
| | | Ala-Lys-Tag2 | 4233 |
| | | Tag1-Glu-Arg- | 1006 |
| | | Tag1-Asn- | 4956 |
| | | Asn-Lys-Tag2 | 4207 |
| | | Asp-Asp-Lys-Tag2 | 565 |
| | | Ile-Val-Lys-Tag2 | 642 |
| | | Tag1-Asn-Phe-Leu- | 91 |
| | | Asn-Gln-Lys-Tag2 | 286 |
| 9 | 38 epitopes | Tag1-His- | 3414 |
| | 1-3 amino acids plus | Tag1-Ser-Leu- | 1910 |
| | the tag | Tag1-Met- | 3044 |
| | | Tag1-Cys- | 2440 |
| | | Tag1-Leu-Gly- | 1085 |
| | | Tag1-Tyr- | 4329 |
| | | Tag1-Asn-Leu- | 1429 |
| | | Tyr-Lys-Tag2 | 3314 |
| | | Tag1-Trp- | 2258 |
| | | Tag1-Glu-Ile- | 1285 |
| | | Leu-Lys-Tag2 | 4976 |
| | | His-Lys-Tag2 | 2388 |
| | | Asp-Lys-Tag2 | 3940 |
| | | Tag1-Gly-Ser- | 953 |
| | | Gln-Lys-Tag2 | 3533 |
| | | Tag1-Ala-Val- | 833 |
| | | Tag1-Asn- | 4956 |
| | | Tag1-Thr-Leu- | 1459 |
| | | Met-Lys-Tag2 | 2741 |
| | | Val-Lys-Tag2 | 4249 |
| | | Ser-Ser-Lys-Tag2 | 1363 |
| | | Tag1-Ser-Glu- | 955 |
| | | Tag1-Ile-Leu- | 1691 |
| | | Tag1-Ala- | 4770 |
| | | Gly-Lys-Tag2 | 4167 |
| | | IIe-Lys-Tag2 | 4370 |
| | | Tag1-Glu-Asn- | 1090 |
| | | Tag1-Leu-Ser- | 1823 |
| | | Tag1-Thr- | 4948 |
| | | Tag1-Phe-Ile- | 832 |
| | | Ser-Lys-Tag2 | 4837 |
| | | Asp-Asn-Lys-Tag2 | 531 |
| | | Tag1-Ala-Ser- | 992 |
| | | Asn-Gln-Lys-Tag2 | 286 |
| | | Tag1-Glu-Val- | 972 |
| | | Tag1-Tyr-Gly- | 620 |
| | | Asp-Asp-Lys-Tag2 | 565 |
| | | Tag1-Asn-Phe-Leu- | 91 |
| 10 | 38 epitopes | Tag1-Ser-Leu- | 1910 |
| | 1-3 amino acids plus | Leu-Lys-Tag2 | 4976 |
| | the tag | Gln-Lys-Tag2 | 3533 |
| | | Tag1-Thr-Leu- | 1459 |
| | | Tag1-Ile-Leu- | 1691 |
| | | Tag1-Glu-Asn- | 1090 |
| | | Tag1-Leu-Ser- | 1823 |
| | | Ser-Lys-Tag2 | 4837 |
| | | Tag1-His- | 3414 |
| | | Tyr-Lys-Tag2 | 3314 |
| | | Tag1-Met- | 3044 |
| | | Tag1-Gly-Ser- | 953 |
| | | Tag1-Phe- | 4606 |
| | | Tag1-Ala- | 4770 |
| | | Tag1-Trp- | 2258 |
| | | Tag1-Leu-Leu- | 2161 |
| | | Cys-Lys-Tag2 | 1607 |
| | | Tag1-Ala-Pro- | 488 |
| | | Ile-Val-Lys-Tag2 | 642 |
| | | Met-Lys-Tag2 | 2741 |
| | | Ile-Lys-Tag2 | 4370 |
| | | Tag1-Asn-Leu- | 1429 |
| | | Asp-Lys-Tag2 | 3940 |
| | | Tag1-Asn-Ser- | 1287 |
| | | Val-Lys-Tag2 | 4249 |
| | | Pro-Lys-Tag2 | 3694 |
| | | Tag1-Cys- | 2440 |
| | | Tag1-Ser-Glu- | 955 |
| | | Tag1-Gly-Leu- | 1101 |
| | | Tag1-Arg- | 4963 |
| | | Tag1-Asn-Val- | 954 |
| | | Tag1-Leu-Pro- | 1021 |
| | | Asp-Asn-Lys-Tag2 | 531 |
| | | Tag1-Thr- | 4948 |
| | | Tag1-Ala-Ala- | 909 |
| | | Tag1-Met-Phe-Glu- | 26 |
| | | Tag1-Ser-Pro- | 805 |
| | | Tag1-Asn-Phe-Leu- | 91 |
| 11 | 38 epitopes | Pro-Lys-Tag2 | 3694 |
| | 1-3 amino acids plus | Met-Lys-Tag2 | 2741 |
| | the tag | Tag1-Asp-Ile- | 1165 |
| | | Tyr-Lys-Tag2 | 3314 |
| | | His-Lys-Tag2 | 2388 |
| | | Asp-Lys-Tag2 | 3940 |
| | | Tag1-Cys- | 2440 |
| | | Ser-Lys-Tag2 | 4837 |
| | | Tag1-Met- | 3044 |
| | | Tag1-Glu-Asn- | 1090 |
| | | Asp-Asn-Lys-Tag2 | 531 |
| | | Val-Lys-Tag2 | 4249 |
| | | Tag1-Tyr-Phe- | 577 |
| | | Tag1-Asp-Ser- | 1086 |
| | | Leu-Lys-Tag2 | 4976 |
| | | Tag1-Ser-Ser- | 1768 |
| | | Asn-Lys-Tag2 | 4207 |
| | | Tag1-Leu-Leu- | 2161 |
| | | Tag1-Leu-Ser- | 1823 |
| | | Cys-Lys-Tag2 | 1607 |
| | | Tag1-Asn-Leu- | 1429 |
| | | Tag1-Phe- | 4606 |
| | | Tag1-Ile-Leu- | 1691 |
| | | Tag1-Ser-Glu- | 955 |
| | | Tag1-Asn- | 4956 |
| | | Tag1-Ser- | 5298 |
| | | Phe-Lys-Tag2 | 3782 |
| | | Tag1-Thr-Leu- | 1459 |
| | | Gln-Lys-Tag2 | 3533 |
| | | Asn-Glu-Lys-Tag2 | 794 |
| | | Tag1-His- | 3414 |
| | | Tag1-Ser-His- | 529 |
| | | Tag1-Trp- | 2258 |
| | | Tag1-Tyr-Ala- | 517 |
| | | Tag1-Ser-Pro- | 805 |
| | | Tag1-Thr-Gly- | 792 |
| | | Ala-Ala-Lys-Tag2 | 708 |
| | | Tag1-Asn-Phe-Leu- | 91 |
| 12 | 38 epitopes | Phe-Lys-Tag2 | 3782 |
| | 1-3 amino acids plus | Tag1-Ile-Leu- | 1691 |
| | the tag | Met-Lys-Tag2 | 2741 |
| | | Tyr-Lys-Tag2 | 3314 |
| | | Tag1-Gly-Ser- | 953 |
| | | Cys-Lys-Tag2 | 1607 |
| | | Tag1-Leu-Ser- | 1823 |
| | | Tag1-Glu- | 4977 |
| | | Tag1-Ser-Glu- | 955 |
| | | Tag1-Ser-Leu- | 1910 |
| | | Val-Lys-Tag2 | 4249 |
| | | Tag1-Met- | 3044 |
| | | Tag1-Thr-Leu- | 1459 |
| | | Tag1-Leu-Thr- | 1233 |
| | | Tag1-Asn- | 4956 |
| | | Tag1-Asp-Leu- | 1422 |
| | | Tag1-Arg- | 4963 |
| | | Tag1-Glu-Asn- | 1090 |
| | | His-Lys-Tag2 | 2388 |
| | | Tag1-His- | 3414 |
| | | Ile-Thr-Lys-Tag2 | 702 |
| | | Ser-Lys-Tag2 | 4837 |
| | | Trp-Lys-Tag2 | 1788 |
| | | Asp-Lys-Tag2 | 3940 |
| | | Asp-Asp-Lys-Tag2 | 565 |
| | | Asp-Asn-Lys-Tag2 | 531 |
| | | Leu-Lys-Tag2 | 4976 |
| | | Thr-Lys-Tag2 | 4245 |
| | | Tag1-Cys- | 2440 |
| | | Gln-Lys-Tag2 | 3533 |
| | | Tag1-Trp- | 2258 |
| | | Gly-Lys-Tag2 | 4167 |
| | | Tag1-Ala-Val- | 833 |
| | | Ile-Lys-Tag2 | 4370 |
| | | Tag1-Asn-Phe-Leu- | 91 |
| | | Tag1-Met-Phe-Glu- | 26 |
| | | Tag1-Asn-Tyr-His- | 37 |
| | | Ile-Val-Lys-Tag2 | 642 |
| 13 | 38 epitopes | Tyr-Lys-Tag2 | 3314 |
| | 1-3 amino acids plus | Tag1-Cys- | 2440 |
| | the tag | Tag1-Tyr- | 4329 |
| | | Tag1-Leu-Leu- | 2161 |
| | | Tag1-Asn-Leu- | 1429 |
| | | Tag1-Glu-Asn- | 1090 |
| | | Thr-Lys-Tag2 | 4245 |
| | | Tag1-Thr- | 4948 |
| | | Tag1-Leu-Ser- | 1823 |
| | | Met-Lys-Tag2 | 2741 |
| | | Tag1-Gly-Ser- | 953 |
| | | Phe-Lys-Tag2 | 3782 |
| | | Tag1-Glu-Ile- | 1285 |
| | | Leu-Lys-Tag2 | 4976 |
| | | Val-Lys-Tag2 | 4249 |
| | | Tag1-Trp- | 2258 |
| | | Tag1-Ser-Glu- | 955 |
| | | Tag1-Ser-Leu- | 1910 |
| | | Tag1-Ser-Asn- | 1138 |
| | | Tag1-His- | 3414 |
| | | Tag1-Asn- | 4956 |
| | | Tag1-Ala-Ala- | 909 |
| | | Pro-Lys-Tag2 | 3694 |
| | | Tag1-Asp-Ser- | 1086 |
| | | Ile-Val-Lys-Tag2 | 642 |
| | | Tag1-Met- | 3044 |
| | | Gln-Lys-Tag2 | 3533 |
| | | Tag1-Leu-Gly- | 1085 |
| | | Asp-Lys-Tag2 | 3940 |
| | | Gly-Lys-Tag2 | 4167 |
| | | Tag1-Glu-Leu- | 1765 |
| | | Leu-Ile-Lys-Tag2 | 1030 |
| | | Tag1-Gln- | 4405 |
| | | Asp-Tyr-Lys-Tag2 | 374 |
| | | Tag1-Ile-Leu- | 1691 |
| | | Tag1-Ser-Pro- | 805 |
| | | Val-Phe-Lys-Tag2 | 414 |
| | | Tag1-Asn-Phe-Leu- | 91 |
| 14 | 38 epitopes | Tag1-Cys- | 2440 |
| | 1-3 amino acids plus | Pro-Lys-Tag2 | 3694 |
| | the tag | Tag1-Gln- | 4405 |
| | | Tyr-Lys-Tag2 | 3314 |
| | | Tag1-His- | 3414 |
| | | Tag1-Leu-Leu- | 2161 |
| | | Gln-Lys-Tag2 | 3533 |
| | | Tag1-Leu-Ser- | 1823 |
| | | Val-Lys-Tag2 | 4249 |
| | | Cys-Lys-Tag2 | 1607 |
| | | Ser-Lys-Tag2 | 4837 |
| | | Tag1-Ser-Leu- | 1910 |
| | | Tag1-Asn-Leu- | 1429 |
| | | Ile-Lys-Tag2 | 4370 |
| | | Tag1-Met- | 3044 |
| | | Tag1-Thr-Leu- | 1459 |
| | | Ser-Ser-Lys-Tag2 | 1363 |
| | | Tag1-Asn- | 4956 |
| | | Tag1-Ala-Val- | 833 |
| | | Asp-Asn-Lys-Tag2 | 531 |
| | | Tag1-Phe- | 4606 |
| | | Leu-Lys-Tag2 | 4976 |
| | | Tag1-Ser-Pro- | 805 |
| | | Tag1-Asp-Ser- | 1086 |
| | | Met-Lys-Tag2 | 2741 |
| | | Tag1-Ile-Leu- | 1691 |
| | | His-Lys-Tag2 | 2388 |
| | | Tag1-Asp-Ile- | 1165 |
| | | Tag1-Leu-Gly- | 1085 |
| | | Tag1-Thr- | 4948 |
| | | Tag1-Glu-Asn- | 1090 |
| | | Phe-Lys-Tag2 | 3782 |
| | | Tag1-Asp-Glu- | 1034 |
| | | Tag1-Glu-Ile- | 1285 |
| | | Asp-Asp-Lys-Tag2 | 565 |
| | | Tag1-Tyr-Gly- | 620 |
| | | Tag1-Ala-Gly- | 613 |
| | | Tag1-Asn-Phe-Leu- | 91 |
| 15 | 38 epitopes | Met-Lys-Tag2 | 2741 |
| | 1-3 amino acids plus | Val-Lys-Tag2 | 4249 |
| | the tag | Leu-Lys-Tag2 | 4976 |
| | | Ala-Lys-Tag2 | 4233 |
| | | Leu-Thr-Lys-Tag2 | 1023 |
| | | Cys-Lys-Tag2 | 1607 |
| | | Tag1-Met- | 3044 |
| | | Tyr-Lys-Tag2 | 3314 |
| | | Pro-Lys-Tag2 | 3694 |
| | | Tag1-Phe- | 4606 |
| | | Tag1-Thr- | 4948 |
| | | Phe-Lys-Tag2 | 3782 |
| | | Tag1-Asn-Leu- | 1429 |
| | | Ser-Lys-Tag2 | 4837 |
| | | Thr-Lys-Tag2 | 4245 |
| | | Tag1-Leu-Gly- | 1085 |
| | | Tag1-Ala-Ala- | 909 |
| | | Tag1-Leu-Ser- | 1823 |
| | | Tag1-Ser-Leu- | 1910 |
| | | Tag1-Gly-Ser- | 953 |
| | | Gln-Lys-Tag2 | 3533 |
| | | Tag1-Ser-Glu- | 955 |
| | | Tag1-Asn- | 4956 |
| | | Tag1-Glu-Asn- | 1090 |
| | | Tag1-Asp-Ser- | 1086 |
| | | Tag1-Cys- | 2440 |
| | | Tag1-His- | 3414 |
| | | Tag1-Ala-Leu- | 1357 |
| | | Tag1-Glu-Ile- | 1285 |
| | | Trp-Lys-Tag2 | 1788 |
| | | Tag1-Ile-Leu- | 1691 |
| | | Ile-Lys-Tag2 | 4370 |
| | | Asp-Asn-Lys-Tag2 | 531 |
| | | Tag1-Gln- | 4405 |
| | | Tag1-Gln-Met- | 240 |
| | | Asp-Asp-Lys-Tag2 | 565 |
| | | Ile-Val-Lys-Tag2 | 642 |
| | | Tag1-Asn-Phe-Leu- | 91 |
| 16 | 38 epitopes | Met-Lys-Tag2 | 2741 |
| | 1-3 amino acids plus | Tag1-Trp- | 2258 |
| | the tag | Phe-Lys-Tag2 | 3782 |
| | | Pro-Lys-Tag2 | 3694 |
| | | Tyr-Lys-Tag2 | 3314 |
| | | Tag1-Glu-Ile- | 1285 |
| | | Trp-Lys-Tag2 | 1788 |
| | | Val-Lys-Tag2 | 4249 |
| | | Tag1-Gln- | 4405 |
| | | Tag1-Ser-Ser- | 1768 |
| | | Asn-Lys-Tag2 | 4207 |
| | | Tag1-Asp- | 4856 |
| | | Tag1-Ile-Leu- | 1691 |
| | | Tag1-Ala-Ala- | 909 |
| | | Ile-Val-Lys-Tag2 | 642 |
| | | Tag1-Cys- | 2440 |
| | | Gly-Lys-Tag2 | 4167 |
| | | Tag1-Gly- | 4629 |
| | | Tag1-Asp-Asp- | 921 |
| | | Tag1-Thr- | 4948 |
| | | Tag1-Thr-Pro- | 624 |
| | | Tag1-Ser-Glu- | 955 |
| | | Tag1-Asn- | 4956 |
| | | Tag1-Asn-Val- | 954 |
| | | Tag1-Asp-Ser- | 1086 |
| | | Tag1-Met- | 3044 |
| | | Tag1-His- | 3414 |
| | | Tag1-Ser-Leu- | 1910 |
| | | Tag1-Val-Leu- | 1391 |
| | | Tag1-Ser- | 5298 |
| | | Tag1-Asn-Leu- | 1429 |
| | | Tag1-Asn-Asn- | 1007 |
| | | Tag1-Arg- | 4963 |
| | | Asp-Asn-Lys-Tag2 | 531 |
| | | Tag1-Leu-Pro- | 1021 |
| | | Leu-Lys-Tag2 | 4976 |
| | | Tag1-Gln-Met- | 240 |
| | | Tag1-Asn-Phe-Leu- | 91 |
| 17 | 38 epitopes | Tag1-Cys- | 2440 |
| | 1-3 amino acids plus | Asn-Lys-Tag2 | 4207 |
| | the tag | Tag1-His- | 3414 |
| | | Leu-Lys-Tag2 | 4976 |
| | | Tag1-Trp- | 2258 |
| | | Tag1-Leu-Ser- | 1823 |
| | | Tag1-Phe- | 4606 |
| | | Tag1-Glu- | 4977 |
| | | Tag1-Gly- | 4629 |
| | | Tag1-Gly-Ser- | 953 |
| | | Tag1-Asp-Ser- | 1086 |
| | | Tag1-Ile-Leu- | 1691 |
| | | Tag1-Glu-Ile- | 1285 |
| | | Ala-Lys-Tag2 | 4233 |
| | | Tag1-Ser-Leu- | 1910 |
| | | Met-Lys-Tag2 | 2741 |
| | | His-Lys-Tag2 | 2388 |
| | | Tag1-Glu-Asn- | 1090 |
| | | Tag1-Leu-Glu- | 1326 |
| | | Tag1-Ala- | 4770 |
| | | Tag1-Ser-Glu- | 955 |
| | | Tag1-Gln-Ile- | 741 |
| | | Asp-Asn-Lys-Tag2 | 531 |
| | | Phe-Lys-Tag2 | 3782 |
| | | Tyr-Lys-Tag2 | 3314 |
| | | Pro-Lys-Tag2 | 3694 |
| | | Tag1-Ser-Ser- | 1768 |
| | | Trp-Lys-Tag2 | 1788 |
| | | Tag1-Asn- | 4956 |
| | | Ser-Lys-Tag2 | 4837 |
| | | Leu-Leu-Lys-Tag2 | 1611 |
| | | Tag1-Leu-Phe- | 1045 |
| | | Tag1-Asp-Leu- | 1422 |
| | | Tag1-Ile-Gln- | 656 |
| | | Tag1-Ser-His- | 529 |
| | | Asp-Asp-Lys-Tag2 | 565 |
| | | Tag1-Asn-Phe-Leu- | 91 |
| | | Asn-Gln-Lys-Tag2 | 286 |
| 18 | 38 epitopes | Tag1-Cys- | 2440 |
| | 1-3 amino acids plus | His-Lys-Tag2 | 2388 |
| | the tag | Tag1-Ala- | 4770 |
| | | Tag1-Glu-Asn- | 1090 |
| | | Tag1-Met- | 3044 |
| | | Pro-Lys-Tag2 | 3694 |
| | | Asp-Lys-Tag2 | 3940 |
| | | Met-Lys-Tag2 | 2741 |
| | | Cys-Lys-Tag2 | 1607 |
| | | Tag1-Ser-Leu- | 1910 |
| | | Phe-Lys-Tag2 | 3782 |
| | | Ser-Lys-Tag2 | 4837 |
| | | Ile-Lys-Tag2 | 4370 |
| | | Tag1-Glu-Ile- | 1285 |
| | | Tyr-Lys-Tag2 | 3314 |
| | | Tag1-His- | b3414 |
| | | Tag1-Asn-Leu- | 1429 |
| | | Tag1-Thr-Leu- | 1459 |
| | | Tag1-Asp-Ile- | 1165 |
| | | Leu-Lys-Tag2 | 4976 |
| | | Val-Lys-Tag2 | 4249 |
| | | Tag1-Arg- | 4963 |
| | | Tag1-Thr-Ala- | 833 |
| | | Tag1-Phe- | 4606 |
| | | Tag1-Asn-Val- | 954 |
| | | Tag1-Ser-Glu- | 955 |
| | | Tag1-Leu-Ala- | 1302 |
| | | Tag1-Ala-Ile- | 974 |
| | | Tag1-Ile-Leu- | 1691 |
| | | Tag1-Ser-Pro- | 805 |
| | | Tag1-Thr- | 4948 |
| | | Tag1-Glu-Val- | 972 |
| | | Asp-Tyr-Lys-Tag2 | 374 |
| | | Ala-Leu-Lys-Tag2 | 1007 |
| | | Tag1-Asp-Glu- | 1034 |
| | | Tag1-Ser-His- | 529 |
| | | Asp-Asp-Lys-Tag2 | 565 |
| | | Tag1-Asn-Phe-Leu- | 91 |
| 19 | 38 epitopes | Leu-Lys-Tag2 | 4976 |
| | 1-3 amino acids plus | Tyr-Lys-Tag2 | 3314 |
| | the tag | Tag1-Ile-Leu- | 1691 |
| | | Asn-Lys-Tag2 | 4207 |
| | | Tag1-Asn- | 4956 |
| | | Trp-Lys-Tag2 | 1788 |
| | | Tag1-Ser-Ser- | 1768 |
| | | Tag1-Tyr- | 4329 |
| | | Tag1-His- | 3414 |
| | | Tag1-Glu-Asn- | 1090 |
| | | Tag1-Leu-Gly- | 1085 |
| | | Tag1-Ala-Ala- | 909 |
| | | Met-Lys-Tag2 | 2741 |
| | | Tag1-Leu-Ser- | 1823 |
| | | Cys-Lys-Tag2 | 1607 |
| | | Tag1-Leu-Thr- | 1233 |
| | | Tag1-Gln- | 4405 |
| | | Pro-Lys-Tag2 | 3694 |
| | | Tag1-Asn-Leu- | 1429 |
| | | Asp-Lys-Tag2 | 3940 |
| | | Ala-Lys-Tag2 | 4233 |
| | | Tag1-Ser-Leu- | 1910 |
| | | Tag1-Asp-4856 | 4856 |
| | | Ser-Ile-Lys-Tag2 | 827 |
| | | Tag1-Glu-Ile- | 1285 |
| | | Phe-Lys-Tag2 | 3782 |
| | | Tag1-Cys- | 2440 |
| | | Gly-Lys-Tag2 | 4167 |
| | | Tag1-Asp-Glu- | 1034 |
| | | Tag1-Leu-Leu- | 2161 |
| | | Tag1-Ser- | 5298 |
| | | Asp-Tyr-Lys-Tag2 | 374 |
| | | Tag1-Ala-Gly- | 613 |
| | | Asp-Asp-Lys-Tag2 | 565 |
| | | Ile-Val-Lys-Tag2 | 642 |
| | | Tag1-Ser-Pro- | 805 |
| | | Tag1-Asp-Ser- | 1086 |
| | | Tag1-Asn-Phe-Leu- | 91 |

| Solution | | |
|---|---|---|
| No. | Criteria | Solution |
| 21 | 41 epitopes | Pro-Lys-Hap2 |
| | 2-3 amino acids plus the | Thr-Lys-Hap2 |
| | tag. | Glu-Lys-Hap2 |
| | | Asp-Lys-Hap2 |
| | | Ala-Lys-Hap2 |
| | | Val-Lys-Hap2 |
| | | Phe-Lys-Hap2 |
| | | Ile-Lys-Hap2 |
| | | Tyr-Lys-Hap2 |
| | | Leu-Leu-Lys- |
| | | Hap2 |
| | | Asp-Asn-Lys- |
| | | Hap2 |
| | | Hap1-Leu-Leu- |
| | | Hap1-Asp-Ser- |
| | | Hap1-Ser-Leu- |
| | | Met-Lys-Hap2 |
| | | Hap1-Ser-Ile- |
| | | Ser-Lys-Hap2 |
| | | Hap1-Val-Leu- |
| | | His-Lys-Hap2 |
| | | Hap1-Asn-Val- |
| | | Asn-Lys-Hap2 |
| | | Hap1-Leu-Ser- |
| | | Gly-Lys-Hap2 |
| | | Hap1-Ser-Glu |
| | | Hap1-Ile-Leu- |
| | | Ile-Val-Lys-Hap2 |
| | | Hap1-Asn-Leu- |
| | | Hap1-Phe-Tyr- |
| | | Hap1-Val-Ser- |
| | | Hap1-Thr-Val- |
| | | Hap1-Ser-Arg- |
| | | Hap1-Leu-Thr- |
| | | Trp-Lys-Hap2 |
| | | Leu-Lys-Hap2 |
| | | Hap1-Gly-Ser- |
| | | Hap1-Leu-Pro- |
| | | Hap1-Glu-Ile- |
| | | Hap1-Glu-Asn- |
| | | Hap1-Ser-Ala- |
| | | Arg- |
| | | Hap1-His-Ile- |
| | | Hap1-Asn-Phe- |
| | | Leu- |
| 22 | 41 epitopes | Glu-Lys-Hap2 |
| | 2-3 amino acids plus the | Ile-Lys-Hap2 |
| | tag. | Leu-Lys-Hap2 |
| | | Thr-Lys-Hap2 |
| | | Ala-Lys-Hap2 |
| | | Hap1-Leu-Leu- |
| | | Hap1-Gly-Ser- |
| | | Ser-Lys-Hap2 |
| | | Hap1-Leu-Ser- |
| | | Pro-Lys-Hap2 |
| | | Hap1-Thr-Leu- |
| | | Tyr-Lys-Hap2 |
| | | Hap1-Asn-Leu- |
| | | Met-Lys-Hap2 |
| | | Phe-Lys-Hap2 |
| | | Hap1-Tyr-Phe- |
| | | Gln-Lys-Hap2 |
| | | Asp-Asn-Lys- |
| | | Hap2 |
| | | Hap1-Asn-Val- |
| | | Hap1-Ile-Ser- |
| | | Val-Lys-Hap2 |
| | | Hap1-Gly-Ile- |
| | | Hap1-Ala-His- |
| | | Hap1-Asp-Ser- |
| | | Gly-Lys-Hap2 |
| | | Hap1-Gly-Leu- |
| | | His-Lys-Hap2 |
| | | Hap1-Ile-Leu- |
| | | Hap1-Ser-Arg- |
| | | Ile-Val-Lys-Hap2 |
| | | Hap1-Ser-Glu- |
| | | Asn-Lys-Hap2 |
| | | Hap1-Glu-Asn- |
| | | Asp-Lys-Hap2 |
| | | Hap1-Gln-Ile- |
| | | Hap1-Ala-Pro- |
| | | Hap1-His-Leu- |
| | | Hap1-Phe-Leu- |
| | | Pro- |
| | | Hap1-Gln-Met- |
| | | Thr- |
| | | Hap1-Asp-Ile- |
| | | Hap1-Asn-Phe- |
| | | Leu- |
| 23 | 41 epitopes | Ile-Lys-Hap2 |
| | 2-3 amino acids plus the | Hap1-Gly-Ser- |
| | tag. | Glu-Lys-Hap2 |
| | | Tyr-Lys-Hap2 |
| | | Hap1-Asn-Val- |
| | | Thr-Lys-Hap2 |
| | | Phe-Lys-Hap2 |
| | | Val-Lys-Hap2 |
| | | Ser-Lys-Hap2 |
| | | Asp-Lys-Hap2 |
| | | Hap1-Leu-Thr- |
| | | Hap1-Asn-Leu- |
| | | Hap1-Ser-Arg- |
| | | Pro-Lys-Hap2 |
| | | Ala-Lys-Hap2 |
| | | Hap1-Ile-Leu- |
| | | Leu-Leu-Lys |
| | | Hap2 |
| | | Hap1-Val-Leu- |
| | | Met-Lys-Hap2 |
| | | Hap1-Ser-Leu- |
| | | Trp-Lys-Hap2 |
| | | Hap1-Leu-Leu- |
| | | Leu-Lys-Hap2 |
| | | Asp-Asn-Lys |
| | | Hap2 |
| | | Asn-Lys-Hap2 |
| | | Gly-Lys-Hap2 |
| | | Hap1-Leu-Gly- |
| | | Hap1-Tyr-Gly- |
| | | Hap1-Ser-Glu- |
| | | Hap1-Leu-Ser- |
| | | Hap1-Ser-Ile- |
| | | Hap1-Thr-Val- |
| | | Hap1-Gly-Leu- |
| | | Hap1-Leu-Pro- |
| | | Hap1-Val-Ser- |
| | | Hap1-Gln-Ile- |
| | | Hap1-Glu-Ile- |
| | | Asn-Gln-Lys- |
| | | Hap2 |
| | | Cys-Pro-Lys |
| | | Hap2 |
| | | Hap1-Asp-Ser |
| | | Hap1-Asn-Phe- |
| | | Leu- |
| 24 | 41 epitopes | Ile-Lys-Hap2 |
| | 2-3 amino acids plus the | Gln-Lys-Hap2 |
| | tag. | Tyr-Lys-Hap2 |
| | | Leu-Lys-Hap2 |
| | | Hap1-Leu-Leu- |
| | | Asn-Lys-Hap2 |
| | | Hap1-Leu-Ser- |
| | | Hap1-Leu-Thr- |
| | | Met-Lys-Hap2 |
| | | Val-Lys-Hap2 |
| | | Hap1-Leu-Pro- |
| | | Pro-Lys-Hap2 |
| | | Thr-Lys-Hap2 |
| | | Hap1-Ile-Leu- |
| | | Hap1-Asn-Leu- |
| | | Hap1-Gly-Ser- |
| | | Cys-Lys-Hap2 |
| | | Ala-Lys-Hap2 |
| | | Hap1-Thr-Leu- |
| | | Ser-Lys-Hap2 |
| | | Asp-Lys-Hap2 |
| | | Hap1-Ser-His- |
| | | Trp-Lys-Hap2 |
| | | Glu-Lys-Hap2 |
| | | Hap1-Glu-Val |
| | | Hap1-Val-Ser- |
| | | Hap1-Asn-Val- |
| | | Asp-Asn-Lys- |
| | | Hap2 |
| | | Gly-Lys-Hap2 |
| | | Hap1-Ser-Arg- |
| | | Phe-Lys-Hap2 |
| | | Hap1-Glu-Thr- |
| | | Hap1-Ser-Glu- |
| | | Hap1-Tyr-Phe- |
| | | Leu-Leu-Lys- |
| | | Hap2 |
| | | Hap1-Ala-Phe- |
| | | Phe-Ser-Lys-Hap2 |
| | | Hap1-Gln-Thr- |
| | | Asn- |
| | | Hap1-His-Ile- |
| | | Hap1-Asn-Phe- |
| | | Leu- |
| | | Asn-Gln-Lys- |
| | | Hap2 |
| 25 | 41 epitopes | Ser-Lys-Hap2 |
| | 2-3 amino acids plus the | Pro-Lys-Hap2 |
| | tag. | Trp-Lys-Hap2 |
| | | Hap1-Ile-Ser- |
| | | Gly-Lys-Hap2 |
| | | Ala-Lys-Hap2 |
| | | Phe-Lys-Hap2 |
| | | Tyr-Lys-Hap2 |
| | | Gln-Lys-Hap2 |
| | | Met-Lys-Hap2 |
| | | Hap1-Ile-Leu- |
| | | Hap1-Leu-Gly- |
| | | Glu-Lys-Hap2 |
| | | Asp-Asn-Lys- |
| | | Hap2 |
| | | Hap1-Leu-Leu- |
| | | Hap1-Gly-Ser- |
| | | Asp-Lys-Hap2 |
| | | Hap1-Ser-Ile- |
| | | Val-Lys-Hap2 |
| | | Thr-Lys-Hap2 |
| | | Hap1-Glu-Asn- |
| | | Hap1-Val-Leu- |
| | | Leu-Lys-Hap2 |
| | | Asn-Lys-Hap2 |
| | | Hap1-Ser-Arg- |
| | | Hap1-Asn-Leu- |
| | | Hap1-Ser-Leu- |
| | | Hap1-Ser-Glu- |
| | | Asp-Gly-Lys- |
| | | Hap2 |
| | | Hap1-Gln-Met- |
| | | Hap1-His-Ile- |
| | | Ile-Lys-Hap2 |
| | | Hap1-Val-Ser- |
| | | Hap1-Leu-Thr- |
| | | Leu-Leu-Lys- |
| | | Hap2 |
| | | Hap1-Leu-Pro- |
| | | Hap1-Ser-Thr- |
| | | Hap1-Phe-Phe- |
| | | Hap1-Tyr-Gly- |
| | | Hap1-Asp-Ser- |
| | | Hap1-Asn-Phe- |
| | | Leu- |
| 26 | 41 epitopes | Pro-Lys-Hap2 |
| | 2-3 amino acids plus the | His-Lys-Hap2 |
| | tag. | Ile-Lys-Hap2 |
| | | Gly-Lys-Hap2 |
| | | Hap1-Leu-Ser- |
| | | Val-Lys-Hap2 |
| | | Ala-Lys-Hap2 |
| | | Asn-Lys-Hap2 |
| | | Met-Lys-Hap2 |
| | | Phe-Lys-Hap2 |
| | | Hap1-Ser-Arg- |
| | | Hap1-Ala-Ala- |
| | | Ser-Lys-Hap2 |
| | | Hap1-Asn-Leu- |
| | | Hap1-Val-Leu- |
| | | Glu-Lys-Hap2 |
| | | Hap1-Asn-Val- |
| | | Hap1-Leu-Thr- |
| | | Hap1-Ser-Glu- |
| | | Hap1-Val-Ser- |
| | | Hap1-Ser-Ile- |
| | | Asp-Asn-Lys- |
| | | Hap2 |
| | | Gln-Lys-Hap2 |
| | | Asp-Lys-Hap2 |
| | | Hap1-Glu-Asn- |
| | | Hap1-Ser-Thr- |
| | | Leu-Lys-Hap2 |
| | | Hap1-Gly-Ser- |
| | | Hap1-Gln-Ala- |
| | | Tyr-Lys-Hap2 |
| | | Hap1-Ile-Leu- |
| | | Thr-Lys-Hap2 |
| | | Hap1-Ser-Val- |
| | | Phe-Ser-Lys-Hap2 |
| | | Hap1-Ile-Phe- |
| | | Hap1-Leu-Pro- |
| | | Hap1-Met-Phe- |
| | | Glu- |
| | | Hap1-Ser-His- |
| | | Hap1-His-Ile- |
| | | Hap1-Asn-Phe- |
| | | Leu- |
| | | Asn-Gln-Lys- |
| | | Hap2 |
| 27 | 41 epitopes | Pro-Lys-Hap2 |
| | 2-3 amino acids plus the | His-Lys-Hap2 |
| | tag. | Ile-Lys-Hap2 |
| | | Gly-Lys-Hap2 |
| | | Hap1-Leu-Ser- |
| | | Val-Lys-Hap2 |
| | | Ala-Lys-Hap2 |
| | | Asn-Lys-Hap2 |
| | | Met-Lys-Hap2 |
| | | Hap1-Asn-Leu- |
| | | Glu-Lys-Hap2 |
| | | Hap1-Leu-Thr- |
| | | Hap1-Ser-Glu- |
| | | Hap1-Val-Ser- |
| | | Gln-Lys-Hap2 |
| | | Asp-Lys-Hap2 |
| | | Hap1-Glu-Asn- |
| | | Tyr-Lys-Hap2 |
| | | Hap1-Ile-Leu- |
| | | Hap1-Ser-Val- |
| | | Phe-Lys-Hap2 |
| | | Hap1-Ser-Ile |
| | | Hap1-Gly-Ser- |
| | | Thr-Lys-Hap2 |
| | | Ser-Lys-Hap2 |
| | | Leu-Lys-Hap2 |
| | | Hap1-Ser-Arg- |
| | | Hap1-Asn-Val- |
| | | Hap1-Val-Leu- |
| | | Hap1-Leu-Pro- |
| | | Asp-Asn-Lys- |
| | | Hap2 |
| | | Hap1-Ser-Thr- |
| | | Hap1-Ala-Ala- |
| | | Phe-Ser-Lys-Hap2 |
| | | Hap1-Ile-Phe- |
| | | Hap1-Met-Phe- |
| | | Glu- |
| | | Hap1-Ala-Phe- |
| | | Hap1-Ser-Ala- |
| | | Ala- |
| | | Hap1-His-Ile- |
| | | Ile-Val-Lys-Hap2 |
| | | Hap1-Asn-Phe- |
| | | Leu- |
| 28 | 40 epitopes | Trp-Lys-Hap2 |
| | 2-3 amino acids plus the | Ser-Lys-Hap2 |
| | tag. | Val-Lys-Hap2 |
| | Ile = Leu | Ile-Lys-Hap2 |
| | | Ala-Lys-Hap2 |
| | | Pro-Lys-Hap2 |
| | | Gly-Lys-Hap2 |
| | | Hap1-Ile-Ile- |
| | | Asn-Lys-Hap2 |
| | | Glu-Lys-Hap2 |
| | | Tyr-Lys-Hap2 |
| | | Met-Lys-Hap2 |
| | | Hap1-Ile-Ser |
| | | Phe-Lys-Hap2 |
| | | Hap1-Ser-Ile- |
| | | Ile-Thr-Lys-Hap2 |
| | | Hap1-Glu-Asn- |
| | | Asp-Lys-Hap2 |
| | | Val-Ile-Lys-Hap2 |
| | | Hap1-Tyr-Ile- |
| | | Hap1-Gly-Ser- |
| | | Hap1-Asp-Ser- |
| | | Hap1-Cys-Ile- |
| | | Hap1-Ile-Phe- |
| | | Hap1-Phe-Val- |
| | | Hap1-Ile-Gln- |
| | | Thr-Lys-Hap2 |
| | | Hap1-Ile-Thr- |
| | | Hap1-Tyr-Phe- |
| | | Hap1-Asn-Val- |
| | | Hap1-Ser-Pro- |
| | | His-Lys-Hap2 |
| | | Phe-Ser-Lys-Hap2 |
| | | Asp-Tyr-Lys- |
| | | Hap2 |
| | | Hap1-Glu-Ile-Ser- |
| | | Hap1-Asn-Phe- |
| | | Ile- |
| | | Asp-Asp-Lys- |
| | | Hap2 |
| | | Hap1-His-Ser- |
| | | Hap1-Ser-His- |
| | | Hap1-His-Ile- |
| 29 | 40 epitopes | Trp-Lys-Hap2 |
| | 2-3 amino acids plus the | Ser-Lys-Hap2 |
| | tag. | Val-Lys-Hap2 |
| | Ile = Leu | Ile-Lys-Hap2 |
| | | Ala-Lys-Hap2 |
| | | Pro-Lys-Hap2 |
| | | Gly-Lys-Hap2 |
| | | Hap1-Ile-Ile- |
| | | Asn-Lys-Hap2 |
| | | Glu-Lys-Hap2 |
| | | Met-Lys-Hap2 |
| | | Hap1-Ile-Ser- |
| | | Phe-Lys-Hap2 |
| | | Hap1-Ser-Ile- |
| | | Ile-Thr-Lys-Hap2 |
| | | Asp-lys-Hap2 |
| | | Val-Ile-Lys-Hap2 |
| | | Hap1-Tyr-Ile- |
| | | Hap1-Asp-Ser- |
| | | Hap1-Cys-Ile- |
| | | Hap1-Ile-Gln- |
| | | Thr-Lys-Hap2 |
| | | Hap1-Ile-Thr- |
| | | Hap1-Asn-Val- |
| | | Hap1-Ser-Pro- |
| | | His-Lys-Hap2 |
| | | Phe-Ser-Lys-Hap2 |
| | | Asp-Asp-Lys- |
| | | Hap2 |
| | | Hap1-Ile-Phe- |
| | | Tyr-Lys-Hap2 |
| | | Hap1-Glu-Asn- |
| | | Hap1-Gly-Ser- |
| | | Hap1-Phe-Val- |
| | | Asp-Tyr-Lys- |
| | | Hap2 |
| | | Hap1-Asn-Phe- |
| | | Ile- |
| | | Hap1-Gly-Glu- |
| | | Hap1-Glu-Ile-Ser- |
| | | Hap1-Ile-Val-Phe- |
| | | Hap1-His-Ile- |
| | | Ile-Val-Lys-Hap2 |
| 30 | 40 epitopes | Ile-Lys-Hap2 |
| | 2-3 amino acids plus the | Pro-Lys-Hap2 |
| | tag. | Ala-Lys-Hap2 |
| | Ile = Leu | Tyr-Lys-Hap2 |
| | | Hap1-Thr-Ile- |
| | | Asp-Lys-Hap2 |
| | | Hap1-Ile-Thr- |
| | | Hap1-Tyr-Ile- |
| | | Ile-Ile-Lys-Hap2 |
| | | Hap1-Ile-Ile- |
| | | Hap1-Gly-Ser- |
| | | Glu-Lys-Hap2 |
| | | Hap1-Thr-Ser- |
| | | Met-Lys-Hap2 |
| | | Ser-Lys-Hap2 |
| | | Val-Lys-Hap2 |
| | | Asn-Lys-Hap2 |
| | | Hap1-Phe-Ser- |
| | | Hap1-Ile-Phe- |
| | | Phe-Ile-Lys-Hap2 |
| | | Phe-Lys-Hap2 |
| | | Hap1-Ser-Ile- |
| | | Hap1-Ile-Ala- |
| | | Gly-Lys-Hap2 |
| | | Hap1-Ser-Glu- |
| | | Hap1-Ile-Gln- |
| | | Hap1-Ser-Pro- |
| | | Thr-Lys-Hap2 |
| | | Asp-Asn-Lys- |
| | | Hap2 |
| | | Hap1-Val-Ile- |
| | | Hap1-Ile-Pro- |
| | | Hap1-Glu-Asn- |
| | | Hap1-Asn-Val- |
| | | Hap1-Gly-Glu- |
| | | Hap1-Asn-Gln- |
| | | Asn- |
| | | Hap1-Asp-Ile-Ile- |
| | | Hap1-Asp-Ser- |
| | | Hap1-His-Ile- |
| | | Ile-Val-Lys-Hap2 |
| | | Hap1-Asn-Phe- |
| | | Ile- |
| 31 | 40 epitopes | Pro-Lys-Hap2 |
| | 2-3 amino acids plus the | Ala-Lys-Hap2 |
| | tag. | Asp-Lys-Hap2 |
| | Ile = Leu | Hap1-Ile-Thr- |
| | | Ile-Ile-Lys-Hap2 |
| | | Hap1-Ile-Ile- |
| | | Hap1-Gly-Ser- |
| | | Glu-Lys-Hap2 |
| | | Hap1-Thr-Ser- |
| | | Ser-Lys-Hap2 |
| | | Val-Lys-Hap2 |
| | | Phe-Ile-Lys-Hap2 |
| | | Phe-Lys-Hap2 |
| | | Hap1-Ser-Ile- |
| | | Hap1-Ile-Ala- |
| | | Gly-Lys-Hap2 |
| | | Hap1-Ser-Glu- |
| | | Hap1-Ser-Pro- |
| | | Asp-Asn-Lys- |
| | | Hap2 |
| | | Hap1-Val-Ile- |
| | | Hap1-Glu-Asn- |
| | | Hap1-Tyr-Ile- |
| | | Ile-Lys-Hap2 |
| | | Hap1-Thr-Ile- |
| | | Tyr-Lys-Hap2 |
| | | Hap1-Ile-Phe- |
| | | Hap1-Ile-Gln- |
| | | Hap1-Phe-Ser- |
| | | Hap1-Ala-Ala- |
| | | Asn-Lys-Hap2 |
| | | Hap1-Ile-Pro- |
| | | Thr-Lys-Hap2 |
| | | Met-Lys-Hap2 |
| | | Hap1-Asn-Val- |
| | | Hap1-Gly-Glu- |
| | | Hap1-Gly-Thr-Ile- |
| | | Hap1-Asp-Ser- |
| | | Hap1-His-Ile- |
| | | Ile-Val-Lys-Hap2 |
| | | Hap1-Asn-Phe- |
| | | Ile- |
| 32 | 40 epitopes | Pro-Lys-Hap2 |
| | 2-3 amino acids plus the | Ala-Lys-Hap2 |
| | tag. | Asp-Lys-Hap2 |
| | Ile = Leu | Ile-Ile-Lys-Hap2 |
| | | Hap1-Ile-Ile- |
| | | Hap1-Gly-Ser- |
| | | Glu-Lys-Hap2 |
| | | Phe-Ile-Lys-Hap2 |
| | | Hap1-Ser-Ile- |
| | | Hap1-Ile-Ala- |
| | | Gly-Lys-Hap2 |
| | | Hap1-Ser-Glu- |
| | | Hap1-Ser-Pro- |
| | | Hap1-Val-Ile- |
| | | Hap1-Tyr-Ile- |
| | | Ile-Lys-Hap2 |
| | | Hap1-Thr-Ile- |
| | | Hap1-Ile-Gln- |
| | | Asn-Lys-Hap2 |
| | | Thr-Lys-Hap2 |
| | | Hap1-Asn-Val- |
| | | Val-Lys-Hap2 |
| | | Hap1-Thr-Ser- |
| | | Asp-Tyr-Lys- |
| | | Hap2 |
| | | Hap1-Ile-Pro- |
| | | Tyr-Lys-Hap2 |
| | | Hap1-Ala-Ala- |
| | | Phe-Lys-Hap2 |
| | | Hap1-Ile-Phe- |
| | | Ser-Lys-Hap2 |
| | | Met-Lys-Hap2 |
| | | Hap1-Glu-Asn- |
| | | Hap1-Ile-Thr- |
| | | Hap1-Phe-Ser- |
| | | Hap1-Gly-Glu- |
| | | Hap1-Gly-Thr-Ile- |
| | | Hap1-Asp-Ser- |
| | | Hap1-His-Ile- |
| | | Ile-Val-Lys-Hap2 |
| | | Hap1-Asn-Phe- |
| | | Ile- |
| 33 | 40 epitopes | Pro-Lys-Hap2 |
| | 2-3 amino acids plus the | Ala-Lys-Hap2 |
| | tag. | Ile-Ile-Lys-Hap2 |
| | Ile = Leu | Hap1-Ile-Ile- |
| | | Hap1-Gly-Ser- |
| | | Glu-Lys-Hap2 |
| | | Phe-Ile-Lys-Hap2 |
| | | Hap1-Ser-Ile- |
| | | Hap1-Ser-Pro- |
| | | Hap1-Val-Ile- |
| | | Hap1-Tyr-Ile- |
| | | Ile-Lys-Hap2 |
| | | Hap1-Thr-Ile- |
| | | Asn-Lys-Hap2 |
| | | Thr-Lys-Hap2 |
| | | Hap1-Asn-Val- |
| | | Val-Lys-Hap2 |
| | | Asp-Tyr-Lys- |
| | | Hap2 |
| | | Hap1-Ile-Pro- |
| | | Tyr-Lys-Hap2 |
| | | Phe-Lys-Hap2 |
| | | Hap1-Ile-Phe |
| | | Ser-Lys-Hap2 |
| | | Met-Lys-Hap2 |
| | | Hap1-Glu-Asn- |
| | | Hap1-Ile-Thr- |
| | | Hap1-Phe-Ser- |
| | | Hap1-His-Ile- |
| | | Hap1-Ile-Ala- |
| | | Asp-Lys-Hap2 |
| | | Hap1-Ile-Gln- |
| | | Hap1-Thr-Ser- |
| | | Gly-Lys-Hap2 |
| | | Hap1-Gly-Glu- |
| | | Hap1-Ser-Glu- |
| | | Hap1-Glu-Val- |
| | | Gly- |
| | | Hap1-Ala-Ala- |
| | | Hap1-Asp-Ile-Ile- |
| | | Ile-Val-Lys-Hap2 |
| | | Hap1-Asn-Phe- |
| | | Ile- |
| 34 | 40 epitopes | Glu-Lys-Hap2 |
| | 2-3 amino acids plus the | Hap1-Ser-Ile- |
| | tag. | Hap1-Val-Ile- |
| | Ile = Leu | Hap1-Tyr-Ile- |
| | | Hap1-Ile-Pro- |
| | | Hap1-Ile-Phe- |
| | | Ser-Lys-Hap2 |
| | | Hap1-Ile-Ala- |
| | | Ala-Lys-Hap2 |
| | | Ile-Lys-Hap2 |
| | | Val-Lys-Hap2 |
| | | Gly-Lys-Hap2 |
| | | Hap1-Thr-Ile- |
| | | Hap1-Gly-Ser- |
| | | Phe-Lys-Hap2 |
| | | Tyr-Lys-Hap2 |
| | | Asp-Lys-Hap2 |
| | | Hap1-Ile-Gln- |
| | | Ile-Ile-Lys-Hap2 |
| | | Hap1-Ser-Pro- |
| | | Hap1-Ile-Thr- |
| | | Hap1-Thr-Ser- |
| | | Pro-Lys-Hap2 |
| | | Asn-Lys-Hap2 |
| | | Phe-Ile-Lys-Hap2 |
| | | Hap1-Ser-Glu- |
| | | Met-Lys-Hap2 |
| | | Hap1-Gly-Glu- |
| | | Hap1-Glu-Asn- |
| | | Thr-Lys-Hap2 |
| | | Hap1-Ser-Ala- |
| | | Hap1-Asn-Val- |
| | | Asp-Tyr-Lys- |
| | | Hap2 |
| | | Hap1-Phe-Ser- |
| | | Hap1-Ala-Pro- |
| | | Phe-Ser-Lys-Hap2 |
| | | Hap1-His-Ile-Arg- |
| | | Hap1-Asp-Ser- |
| | | Ile-Val-Lys-Hap2 |
| | | Hap1-Asn-Phe- |
| | | Ile- |
| 35 | 40 epitopes | Glu-Lys-Hap2 |
| | 2-3 amino acids plus the | Val-Lys-Hap2 |
| | tag. | Tyr-Lys-Hap2 |
| | Ile = Leu | Phe-Lys-Hap2 |
| | | Hap1-Ile-Pro- |
| | | Gly-Lys-Hap2 |
| | | Asp-Lys-Hap2 |
| | | Ile-Lys-Hap2 |
| | | Hap1-Ser-Glu- |
| | | Hap1-Ile-Phe- |
| | | Hap1-Phe-Ser- |
| | | Ile-Ile-Lys-Hap2 |
| | | Phe-Ile-Lys-Hap2 |
| | | Hap1-Ser-Ile- |
| | | Met-Lys-Hap2 |
| | | Hap1-Val-Ile- |
| | | Pro-Lys-Hap2 |
| | | Asn-Lys-Hap2 |
| | | Hap1-Glu-Asn- |
| | | Hap1-Ile-Ala- |
| | | Hap1-Thr-Ser- |
| | | Hap1-Gly-Ser- |
| | | Ala-Lys-Hap2 |
| | | Hap1-Tyr-Ile- |
| | | Hap1-Thr-Ile- |
| | | Hap1-Ile-Gln- |
| | | Hap1-Ile-Thr- |
| | | Asp-Asn-Lys- |
| | | Hap2 |
| | | Hap1-His-Ile- |
| | | Ser-Lys-Hap2 |
| | | Hap1-Ile-Ile- |
| | | Hap1-Ser-Pro- |
| | | Hap1-Asn-Val- |
| | | Thr-Lys-Hap2 |
| | | Hap1-Gly-Glu- |
| | | Hap1-Met-Phe- |
| | | Gly- |
| | | Hap1-Ala-Ala- |
| | | Hap1-Asp-Ile-Ile- |
| | | Ile-Val-Lys-Hap2 |
| | | Hap1-Asn-Phe- |
| | | Ile- |
| 36 | 40 epitopes | Hap1-Ile-Ile- |
| | 2-3 amino acids plus the | Gly-Lys-Hap2 |
| | tag. | Thr-Lys-Hap2 |
| | Ile = Leu | Ser-Lys-Hap2 |
| | | Pro-Lys-Hap2 |
| | | Met-Lys-Hap2 |
| | | Ile-Lys-Hap2 |
| | | Hap1-Ile-Gln- |
| | | Hap1-Thr-Ser- |
| | | Tyr-Lys-Hap2 |
| | | Hap1-Ser-Ile- |
| | | Asn-Lys-Hap2 |
| | | Hap1-Thr-Ile- |
| | | Hap1-Ile-Phe- |
| | | Phe-Lys-Hap2 |
| | | Hap1-Asn-Ile |
| | | Hap1-Ser-Ser- |
| | | Hap1-Tyr-Ile- |
| | | Hap1-Asn-Val- |
| | | Hap1-Ile-Ser- |
| | | Val-Lys-Hap2 |
| | | Hap1-Ser-Val- |
| | | Hap1-Thr-Val- |
| | | Hap1-Gly-Ser- |
| | | Ile-Ile-Lys-Hap2 |
| | | Hap1-Ile-Thr- |
| | | Hap1-Ile-Val-Ala- |
| | | Asp-Asp-Lys- |
| | | Hap2 |
| | | Trp-Lys-Hap2 |
| | | Asp-Lys-Hap2 |
| | | Hap1-Ser-Asn- |
| | | Hap1-Gln-Ile- |
| | | Ala-Lys-Hap2 |
| | | Hap1-Glu-Asn- |
| | | Ser-Ile-Lys-Hap2 |
| | | Ala-Pro-Lys-Hap2 |
| | | Hap1-Met-Phe- |
| | | Gly- |
| | | Hap1-Gln-Met- |
| | | Thr- |
| | | Ile-Val-Lys-Hap2 |
| | | Hap1-Asn-Phe- |
| | | Ile- |
| 37 | 40 epitopes | Gly-Lys-Hap2 |
| | 2-3 amino acids plus the | Thr-Lys-Hap2 |
| | tag. | Pro-Lys-Hap2 |
| | Ile = Leu | Met-Lys-Hap2 |
| | | Ile-Lys-Hap2 |
| | | Hap1-Ile-Gln- |
| | | Hap1-Ser-Ile- |
| | | Phe-Lys-Hap2 |
| | | Hap1-Ser-Ser- |
| | | Hap1-Tyr-Ile- |
| | | Hap1-Ile-Ser- |
| | | Val-Lys-Hap2 |
| | | Hap1-Thr-Val- |
| | | Hap1-Gly-Ser- |
| | | Hap1-Gln-Ile- |
| | | Asp-Lys-Hap2 |
| | | Hap1-Gly-Ile- |
| | | Hap1-Ile-Phe- |
| | | Hap1-Asn-Val- |
| | | Ile-Ile-Lys-Hap2 |
| | | Asp-Asn-Lys- |
| | | Hap2 |
| | | Ser-Lys-Hap2 |
| | | Hap1-Asn-Ile- |
| | | Asn-Lys-Hap2 |
| | | Hap1-Ile-Ile- |
| | | Tyr-Lys-Hap2 |
| | | Asp-Asp-Lys- |
| | | Hap2 |
| | | Hap1-Ser-Asn- |
| | | Ile-Val-Lys-Hap2 |
| | | Hap1-Thr-Ile- |
| | | Ala-Lys-Hap2 |
| | | Hap1-Thr-Ser- |
| | | Hap1-Ser-Val- |
| | | Hap1-Tyr-Val- |
| | | Hap1-Thr-Pro- |
| | | Hap1-Ile-Thr- |
| | | Hap1-Met-Phe- |
| | | Glu- |
| | | Hap1-Ser-Pro- |
| | | Hap1-Ala-Gly- |
| | | Hap1-Asn-Phe- |
| | | Ile- |
| 38 | 40 epitopes | Hap1-Ile-Ile- |
| | 2-3 amino acids plus the | Val-Lys-Hap2 |
| | tag. | Phe-Lys-Hap2 |
| | Ile = Leu | Pro-Lys-Hap2 |
| | | Ile-Lys-Hap2 |
| | | Hap1-Thr-Ile- |
| | | Thr-Lys-Hap2 |
| | | Hap1-Gly-Ser- |
| | | Ala-Lys-Hap2 |
| | | Asn-Lys-Hap2 |
| | | Hap1-Ile-Ser |
| | | Hap1-Ser-Ile- |
| | | Hap1-Gln-Ile- |
| | | Hap1-Asn-Val- |
| | | Hap1-Thr-Val- |
| | | Hap1-Tyr-Ile- |
| | | Hap1-Gly-Ile- |
| | | Hap1-Ile-Phe- |
| | | Hap1-Asn-Ile- |
| | | Tyr-Lys-Hap2 |
| | | Asp-Lys-Hap2 |
| | | Hap1-Ser-Ser- |
| | | Asp-Tyr-Lys- |
| | | Hap2 |
| | | Hap1-Ala-Gly- |
| | | Ile-Ile-Lys-Hap2 |
| | | Hap1-Thr-Ser- |
| | | Hap1-Glu-Asn- |
| | | Hap1-Ile-Thr- |
| | | Ser-Lys-Hap2 |
| | | Met-Lys-Hap2 |
| | | Hap1-Ile-Gln- |
| | | Gly-Lys-Hap2 |
| | | Hap1-Ile-Pro- |
| | | Hap1-Ala-Pro- |
| | | Hap1-Asp-Thr- |
| | | Hap1-Met-Phe- |
| | | Glu- |
| | | Hap1-Ser-Gln- |
| | | Thr- |
| | | Hap1-Asp-Ile-Ile- |
| | | Ile-Val-Lys-Hap2 |
| | | Hap1-Asn-Phe- |
| | | Ile- |
| 39 | 40 epitopes | Phe-Lys-Hap2 |
| | 2-3 amino acids plus the | Ser-Lys-Hap2 |
| | tag. | Asp-Lys-Hap2 |
| | Ile = Leu | Asn-Lys-Hap2 |
| | | Hap1-Ile-Ser- |
| | | Hap1-Val-Ile- |
| | | Hap1-Ile-Ile- |
| | | Pro-Lys-Hap2 |
| | | Ile-Thr-Lys-Hap2 |
| | | Thr-Lys-Hap2 |
| | | Phe-Ile-Lys-Hap2 |
| | | Ile-Lys-Hap2 |
| | | Trp-Lys-Hap2 |
| | | Hap1-Tyr-Ile- |
| | | Ala-Lys-Hap2 |
| | | Hap1-Ser-Ile- |
| | | Ser-Phe-Lys-Hap2 |
| | | Cys-Lys-Hap2 |
| | | Hap1-Ile-Glu- |
| | | IIe-Val-Lys-Hap2 |
| | | Hap1-Ile-Thr- |
| | | Ile-Ser-Lys-Hap2 |
| | | Gly-Lys-Hap2 |
| | | Hap1-Ile-Pro- |
| | | Glu-Lys-Hap2 |
| | | Hap1-Ser-Glu- |
| | | Val-Lys-Hap2 |
| | | Ile-Asp-Lys-Hap2 |
| | | Met-Lys-Hap2 |
| | | Hap1-Asn-Val- |
| | | Phe-Glu-Lys- |
| | | Hap2 |
| | | Hap1-Gly-Ser- |
| | | Tyr-Lys-Hap2 |
| | | Asp-Asn-Lys- |
| | | Hap2 |
| | | Hap1-His-Ile- |
| | | His-Lys-Hap2 |
| | | Hap1-Met-Phe- |
| | | Glu- |
| | | Hap1-Ser-Ala- |
| | | Ala- |
| | | Cys-Ile-Lys- |
| | | Hap2Hap1-Asn- |
| | | Phe-Ile- |
| 40 | 40 epitopes2-3 amino | Phe-Lys-Hap2 |
| | acids plus the tag.Ile = | Ser-Lys-Hap2 |
| | Leu | Asp-Lys-Hap2 |
| | | Asn-Lys-Hap2 |
| | | Hap1-Ile-Ser- |
| | | Hap1-Val-Ile- |
| | | Hap1-Ile-Ile- |
| | | Pro-Lys-Hap2 |
| | | Ile-Thr-Lys-Hap2 |
| | | Thr-Lys-Hap2 |
| | | Phe-Ile-Lys-Hap2 |
| | | Ile-Lys-Hap2 |
| | | Trp-Lys-Hap2 |
| | | Hap1-Tyr-Ile- |
| | | Ala-Lys-Hap2 |
| | | Hap1-Ser-Ile- |
| | | Ser-Phe-Lys-Hap2 |
| | | Cys-Lys-Hap2 |
| | | Hap1-Ile-Glu- |
| | | Ile-Val-Lys-Hap2 |
| | | Hap1-Ile-Thr- |
| | | Ile-Ser-Lys-Hap2 |
| | | Gly-Lys-Hap2 |
| | | Hap1-Ile-Pro- |
| | | Glu-Lys-Hap2 |
| | | Hap1-Ser-Glu- |
| | | Val-Lys-Hap2 |
| | | Ile-Asp-Lys-Hap2 |
| | | Met-Lys-Hap2 |
| | | Hap1-Asn-Val- |
| | | Phe-Glu-Lys- |
| | | Hap2 |
| | | Hap1-Gly-Ser- |
| | | Tyr-Lys-Hap2 |
| | | Asp-Asn-Lys- |
| | | Hap2 |
| | | Hap1-His-Ile- |
| | | His-Lys-Hap2 |
| | | Hap1-Met-Phe- |
| | | Glu- |
| | | Hap1-Ser-Ala- |
| | | Ala- |
| | | Cys-Ile-Lys-Hap2 |
| | | Hap1-Asn-Phe- |
| | | Ile- |
| 41 | 40 epitopes 2-3 amino | Phe-Lys-Hap2 |
| | acids plus the tag. Ile = | Hap1-Thr-Ile- |
| | Leu | Asn-Lys-Hap2 |
| | | Met-Lys-Hap2 |
| | | Hap1-Gly-Ser- |
| | | Hap1-Ile-Phe- |
| | | Hap1-Asn-Val- |
| | | Pro-Lys-Hap2 |
| | | Hap1-Ile-Ile- |
| | | Ile-Lys-Hap2 |
| | | Ser-Lys-Hap2 |
| | | Hap1-Tyr-Ile- |
| | | Val-Lys-Hap2 |
| | | Hap1-Ile-Gln- |
| | | Gly-Lys-Hap2 |
| | | Asp-Lys-Hap2 |
| | | Ala-Lys-Hap2 |
| | | Hap1-Asp-Thr- |
| | | Hap1-Ile-Ser- |
| | | Thr-Lys-Hap2 |
| | | Hap1-Ile-Arg- |
| | | Hap1-Thr-Ser- |
| | | Hap1-Ile-Val-Ala- |
| | | Val-Glu-Lys- |
| | | Hap2 |
| | | Ile-Val-Lys-Hap2 |
| | | Ile-Ile-Lys-Hap2 |
| | | Hap1-Thr-Val- |
| | | Ser-Ile-Lys-Hap2 |
| | | Hap1-Glu-Asn- |
| | | Hap1-Tyr-Ala- |
| | | Hap1-His-Ile- |
| | | Tyr-Lys-Hap2 |
| | | Hap1-Ser-Asn- |
| | | Hap1-Tyr-Phe- |
| | | Hap1-Ala-Ala- |
| | | Hap1-Ser-Ile- |
| | | Glu-Lys-Hap2 |
| | | Hap1-Asp-Ser- |
| | | Hap1-Ser-Pro- |
| | | Thr-Hap1-Asn- |
| | | Phe-Ile- |
| 42 | 40 epitopes | Ser-Lys-Hap2 |
| | 2-3 amino acids plus the | Pro-Lys-Hap2 |
| | tag. | Tyr-Lys-Hap2 |
| | Ile = Leu | Ala-Lys-Hap2 |
| | | Hap1-Ile-Ile- |
| | | Val-Lys-Hap2 |
| | | Hap1-Gly-Ser- |
| | | Asp-Lys-Hap2 |
| | | Ile-Lys-Hap2 |
| | | Glu-Lys-Hap2 |
| | | Hap1-Asn-Ile- |
| | | Gln-Lys-Hap2 |
| | | Cys-Lys-Hap2 |
| | | Thr-Lys-Hap2 |
| | | Hap1-Ser-Thr- |
| | | Met-Lys-Hap2 |
| | | Hap1-His-Ile- |
| | | Hap1-Asn-Val- |
| | | Hap1-Ser-Ser- |
| | | Hap1-Ile-Thr- |
| | | Hap1-Ile-Gln- |
| | | Phe-Lys-Hap2 |
| | | Asp-Asn-Lys |
| | | Hap2 |
| | | Val-Ile-Lys-Hap2 |
| | | Trp-Lys-Hap2 |
| | | Ile-Val-Lys-Hap2 |
| | | Hap1-Ile-Ser |
| | | Hap1-Thr-Ser- |
| | | Hap1-Ser-Pro- |
| | | Hap1-Val-Ser- |
| | | Gly-Lys-Hap2 |
| | | Hap1-Ser-Arg- |
| | | Hap1-Ile-Phe- |
| | | Hap1-Ser-Glu- |
| | | Hap1-Ala-Val- |
| | | Hap1-Ser-Asn- |
| | | Cys-Pro-Lys- |
| | | Hap2 |
| | | Hap1-Met-Phe- |
| | | Glu- |
| | | Asp-Asp-Lys- |
| | | Hap2 |
| | | Hap1-Asn-Phe- |
| | | Ile- |
| 43 | 40 epitopes | Ser-Lys-Hap2 |
| | 2-3 amino acids plus the | Met-Lys-Hap2 |
| | tag. | Phe-Lys-Hap2 |
| | Ile = Leu | Val-Lys-Hap2 |
| | | Tyr-Lys-Hap2 |
| | | Hap1-Ser-Ile- |
| | | Cys-Lys-Hap2 |
| | | Ile-Thr-Lys-Hap2 |
| | | Hap1-Ile-Ser- |
| | | Asp-Lys-Hap2 |
| | | Thr-Lys-Hap2 |
| | | Hap1-Glu-Asn- |
| | | Hap1-Ile-Pro- |
| | | Asn-Lys-Hap2 |
| | | Hap1-Thr-Ile- |
| | | Hap1-Gln-Ile- |
| | | Hap1-Ile-Ile- |
| | | Ala-Lys-Hap2 |
| | | Hap1-Ile-Thr- |
| | | Pro-Lys-Hap2 |
| | | Hap1-Gly-Ser- |
| | | Ile-Lys-Hap2 |
| | | Hap1-Ile-Glu- |
| | | Ser-Ser-Lys-Hap2 |
| | | Ile-Ile-Lys-Hap2 |
| | | Hap1-Asp-Ile-Ile- |
| | | Hap1-Ser-Glu- |
| | | Hap1-Tyr-Gly- |
| | | Hap1-Tyr-Ile- |
| | | Glu-Lys-Hap2 |
| | | Ile-Asp-Lys-Hap2 |
| | | Hap1-Asn-Val- |
| | | Hap1-Tyr-Phe- |
| | | Hap1-Phe-Ser- |
| | | Asp-Asn-Lys- |
| | | Hap2 |
| | | Cys-Ile-Lys-Hap2 |
| | | Hap1-Asn-Phe- |
| | | Ile- |
| | | Asp-Pro-Lys- |
| | | Hap2 |
| | | Hap1-Asp-Ser- |
| | | Hap1-Asn-Tyr- |
| | | His- |
| 44 | 30 epitopes | Hap1-Gly-Ile-Ile- |
| | 2-3 amino acids plus the | Ile-Ser-Lys-Hap2 |
| | tag. | Hap1-Ile-Ala- |
| | Ile = Leu = Val | Asn- |
| | Asp = Glu | Hap1-Asp-Asn- |
| | Asn = Gln | Ile- |
| | Ser = Thr | Hap1-Ile-Phe- |
| | Phe = Trp | Asn- |
| | | Hap1-Phe-Gly-Ile- |
| | | Gly-Asn-Lys- |
| | | Hap2 |
| | | Asp-Asn-Lys- |
| | | Hap2 |
| | | Hap1-Asn-Phe- |
| | | Ser- |
| | | Hap1-Ile-Ser-Phe- |
| | | Hap1-Pro-Ile-Asp- |
| | | Ile-Lys-Lys-Hap2 |
| | | Hap1-Asn-Ile-Ile- |
| | | Hap1-Ser-Ile-Ser- |
| | | Hap1-Ile-Asp-Ile- |
| | | Hap1-Ile-Lys- |
| | | Arg- |
| | | Hap1-Ser-Gly- |
| | | Ser- |
| | | Ile-Phe-Lys-Hap2 |
| | | Hap1-His-Ile-Ser- |
| | | Hap1-Ile-Ile-Arg- |
| | | Hap1-Phe-Ile-Ser- |
| | | Hap1-Ser-Asn- |
| | | Ser- |
| | | Hap1-Ser-Ala-Ile- |
| | | Hap1-Cys-Ile-Ile- |
| | | Hap1-Ile-Ile-His- |
| | | Hap1-Ile-Phe- |
| | | Arg- |
| | | Hap1-Ala-Ile-Arg- |
| | | Hap1-Asp-Ile-Ile- |
| | | Hap1-Ile-Ala-Ser- |
| | | Tyr-Ser-Lys-Hap2 |
| 45 | 77 epitopes | Hap1-Leu-Ala- |
| | 2-3 amino acids plus the | Cys-Lys-Hap2 |
| | tag. | Hap1-Leu-Phe- |
| | All epitopes of solution | Hap1-Ala-Ser- |
| | 20, above, | Hap1-Ala-Ala- |
| | are removed as | Hap1-Ser-Thr- |
| | candidate epitopes | His-Lys-Hap2 |
| | | Phe-Leu-Lys- |
| | | Hap2 |
| | | Hap1-Gly-Ala- |
| | | Hap1-Ser-Ser- |
| | | Hap1-Leu-Ser- |
| | | Hap1-Asp-Gly- |
| | | Hap1-Asp-Thr- |
| | | Hap1-Asp-Leu- |
| | | Leu-Val-Lys- |
| | | Hap2 |
| | | Hap1-Thr-Thr- |
| | | Hap1-Asn-Ser- |
| | | Hap1-Leu-Glu- |
| | | Hap1-Ala-Val- |
| | | Hap1-Val-Ile- |
| | | Hap1-Ala-Leu- |
| | | Phe-Ser-Lys-Hap2 |
| | | Hap1-Asn-Val- |
| | | Hap1-Thr-Leu- |
| | | Hap1-Ile-Thr- |
| | | Hap1-Leu-Gln- |
| | | Hap1-Phe-Leu- |
| | | Hap1-Ser-Gln- |
| | | Hap1-Val-Thr- |
| | | Hap1-Val-Val- |
| | | Hap1-Gln-Leu- |
| | | Hap1-Gly-Leu- |
| | | Hap1-Phe-Ser- |
| | | Val-Ala-Lys-Hap2 |
| | | Hap1-Glu-Arg- |
| | | Leu-Leu-Lys- |
| | | Hap2 |
| | | Hap1-Phe-Phe- |
| | | Hap1-Asn-Ile- |
| | | Hap1-Glu-Val- |
| | | Hap1-Ser-Cys- |
| | | Hap1-Cys-Gly- |
| | | Hap1-Ser-Phe- |
| | | Hap1-Thr-Ser- |
| | | Hap1-Leu-Arg- |
| | | Hap1-Ile-Gln- |
| | | Hap1-Ala-Ile- |
| | | Hap1-Glu-Phe- |
| | | Hap1-Gln-Ser- |
| | | Hap1-Thr-Val- |
| | | Val-Ser-Lys-Hap2 |
| | | Hap1-Leu-Cys- |
| | | Hap1-Ser-Val- |
| | | Hap1-Val-Ala- |
| | | Hap1-Glu-Thr- |
| | | Leu-Thr-Lys- |
| | | Hap2 |
| | | Ile-Ile-Lys-Hap2 |
| | | Hap1-Thr-Gly- |
| | | Ile-Ser-Lys-Hap2 |
| | | Thr-Asp-Lys- |
| | | Hap2 |
| | | Hap1-Ser-Ala- |
| | | Ser-Thr-Lys-Hap2 |
| | | Val-Leu-Lys- |
| | | Hap2 |
| | | Hap1-Ser-Asp- |
| | | Hap1-Ser-Pro- |
| | | Leu-Asp-Lys- |
| | | Hap2 |
| | | Hap1-Gly-Ser- |
| | | Ser- |
| | | Hap1-Leu-Val- |
| | | Leu-Ser-Lys-Hap2 |
| | | Hap1-Thr-Arg- |
| | | Hap1-Val-Phe- |
| | | Hap1-Gln-Ile- |
| | | Hap1-Gln-Thr- |
| | | Hap1-Tyr-Thr |
| | | Hap1-Ile-Val- |
| | | Hap1-Tyr-Ala- |
| | | Hap1-Tyr-Gly- |
| | | Hap1-Ala-Met- |
| 46 | 77 epitopes* | Hap1-Leu-Phe- |
| | 2-3 amino acids plus the | Hap1-Val-Val- |
| | tag. | Hap1-Asp-Leu- |
| | All epitopes of solution | Hap1-Leu-Ser- |
| | 20, above, | Ile-Ser-Lys-Hap2 |
| | are removed as | Hap1-Ala-Ile- |
| | candidate epitopes | Cys-Lys-Hap2 |
| | | Hap1-Ala-Val- |
| | | Hap1-Phe-Phe- |
| | | Hap1-Ile-Phe- |
| | | Leu-Leu-Lys- |
| | | Hap2 |
| | | Phe-Leu-Lys- |
| | | Hap2 |
| | | Hap1-Gly-Ala- |
| | | His-Lys-Hap2 |
| | | Hap1-Leu-Gln- |
| | | Hap1-Ile-Ile- |
| | | Hap1-Thr-Ser- |
| | | Hap1-Ser-Val- |
| | | Hap1-Gly-Thr- |
| | | Hap1-Thr-Pro- |
| | | Hap1-Asp-Gly- |
| | | Hap1-Phe-Leu- |
| | | Hap1-Leu-Arg- |
| | | Hap1-Ser-Pro- |
| | | Hap1-Val-His- |
| | | Hap1-Ser-Thr- |
| | | Hap1-Val-Asn- |
| | | Hap1-Thr-Thr- |
| | | Hap1-Ser-Gly- |
| | | Hap1-Gln-Gln- |
| | | Ser-Ser-Lys-Hap2 |
| | | Hap1-Val-Ala- |
| | | Hap1-Asn-Ser- |
| | | Hap1-Ile-Gln- |
| | | Hap1-Gly-Leu- |
| | | Hap1-Glu-Thr- |
| | | Hap1-Ala-Met- |
| | | Leu-Val-Lys- |
| | | Hap2 |
| | | Hap1-Gln-Ile- |
| | | Hap1-Ser-Ala- |
| | | Hap1-Leu-Val- |
| | | Leu-Asp-Lys- |
| | | Hap2 |
| | | Hap1-Thr-Leu- |
| | | Hap1-Tyr-Ala- |
| | | Hap1-Ser-Ser- |
| | | Hap1-Leu-Ile- |
| | | Hap1-Ala-Ser- |
| | | Hap1-Asp-Thr- |
| | | Hap1-Ala-Ala- |
| | | Hap1-Glu-Phe- |
| | | Hap1-Ala-Leu- |
| | | Hap1-Asn-Ile- |
| | | Hap1-Tyr-Phe- |
| | | Hap1-Thr-Gly- |
| | | Hap1-Leu-Glu- |
| | | Hap1-Leu-Cys- |
| | | Hap1-Ser-Asn- |
| | | Hap1-Asn-Gly- |
| | | Hap1-Asn-Val- |
| | | Hap1-Ala-Gly- |
| | | Hap1-Phe-Ser- |
| | | Hap1-Leu-Ala- |
| | | Hap1-Ser-Phe- |
| | | Hap1-Thr-Gln- |
| | | Hap1-Phe-Tyr- |
| | | Hap1-Asp-Ser- |
| | | Hap1-Thr-Val- |
| | | Hap1-Glu-Ile- |
| | | Hap1-Glu-Arg- |
| | | Hap1-Gln-Thr- |
| | | Hap1-Thr-Tyr- |
| | | Hap1-Val-Thr- |
| | | Cys-Leu-Lys- |
| | | Hap2 |
| | | Hap1-Asn-Pro- |
| | | Hap1-Phe-Pro- |
| | | Hap1-Ile-Leu- |
| | | Glu- |
| | | Ile-Val-Lys-Hap2 |
| 47 | 77 epitopes* | Hap1-Leu-Phe- |
| | 2-3 amino acids plus the | Hap1-Val-Val- |
| | tag. | Hap1-Asp-Leu- |
| | All epitopes of solution | Hap1-Leu-Ser- |
| | 20, above, | Ile-Ser-Lys-Hap2 |
| | are removed as | Hap1-Ala-Ile- |
| | candidate epitopes | Cys-Lys-Hap2 |
| | | Hap1-Ala-Val- |
| | | Hap1-Phe-Phe- |
| | | Hap1-Ile-Phe- |
| | | Leu-Leu-Lys- |
| | | Hap2 |
| | | Hap1-Gly-Ala- |
| | | His-Lys-Hap2 |
| | | Hap1-Leu-Gln- |
| | | Hap1-Ile-Ile- |
| | | Hap1-Thr-Ser- |
| | | Hap1-Ser-Val- |
| | | Hap1-Gly-Thr- |
| | | Hap1-Thr-Pro- |
| | | Hap1-Asp-Gly- |
| | | Hap1-Phe-Leu- |
| | | Hap1-Leu-Arg- |
| | | Hap1-Val-His- |
| | | Hap1-Ser-Thr- |
| | | Hap1-Val-Asn- |
| | | Hap1-Thr-Thr- |
| | | Hap1-Ser-Gly- |
| | | Hap1-Gln-Gln- |
| | | Ser-Ser-Lys-Hap2 |
| | | Hap1-Val-Ala- |
| | | Hap1-Asn-Ser- |
| | | Hap1-Ile-Gln- |
| | | Hap1-Gly-Leu- |
| | | Hap1-Glu-Thr- |
| | | Hap1-Ala-Met- |
| | | Hap1-Gln-Ile- |
| | | Leu-Asp-Lys |
| | | Hap2 |
| | | Hap1-Thr-Leu- |
| | | Hap1-Ser-Ser- |
| | | Hap1-Leu-Ile- |
| | | Hap1-Ala-Ser- |
| | | Hap1-Asp-Thr- |
| | | Hap1-Ala-Ala- |
| | | Hap1-Asn-Ile- |
| | | Hap1-Tyr-Phe- |
| | | Hap1-Thr-Gly |
| | | Hap1-Leu-Glu- |
| | | Hap1-Ser-Asn- |
| | | Hap1-Leu-Ala- |
| | | Hap1-Thr-Gln- |
| | | Hap1-Phe-Tyr- |
| | | Hap1-Asp-Ser- |
| | | Hap1-Thr-Val- |
| | | Hap1-Glu-Ile- |
| | | Hap1-Glu-Arg- |
| | | Hap1-Gln-Thr- |
| | | Hap1-Thr-Tyr- |
| | | Cys-Leu-Lys- |
| | | Hap2 |
| | | Hap1-Phe-Ser- |
| | | Hap1-Ser-Ala- |
| | | Hap1-Leu-Cys- |
| | | Hap1-Tyr-Ala- |
| | | Hap1-Asn-Val- |
| | | Hap1-Phe-Pro- |
| | | Phe-Leu-Lys- |
| | | Hap2 |
| | | Hap1-Ser-Pro- |
| | | Hap1-Leu-Val- |
| | | Hap1-Asn-Gly- |
| | | Leu-Val-Lys- |
| | | Hap2 |
| | | Hap1-Ser-Phe- |
| | | Hap1-Ala-Leu- |
| | | Hap1-Val-Thr- |
| | | Hap1-Ala-Gly- |
| | | Hap1-Glu-Phe- |
| | | Hap1-Asn-Pro- |
| | | Tyr-Gly-Lys- |
| | | Hap2 |
| | | Ile-Val-Lys-Hap2 |
| 48 | 77 epitopes* | Hap1-Val-Val- |
| | 2-3 amino acids plus the | Hap1-Asp-Leu- |
| | tag. | Hap1-Ala-Ile- |
| | All epitopes of solution | Cys-Lys-Hap2 |
| | 20, above, | Hap1-Ala-Val- |
| | are removed as | Hap1-Phe-Phe- |
| | candidate epitopes | Hap1-Ile-Phe- |
| | | Leu-Leu-Lys- |
| | | Hap2 |
| | | Hap1-Gly-Ala- |
| | | His-Lys-Hap2 |
| | | Hap1-Leu-Gln- |
| | | Hap1-Thr-Ser- |
| | | Hap1-Leu-Arg- |
| | | Hap1-Ser-Thr- |
| | | Hap1-Val-Asn- |
| | | Hap1-Thr-Thr- |
| | | Hap1-Ser-Gly- |
| | | Hap1-Gln-Gln- |
| | | Hap1-Asn-Ser- |
| | | Hap1-Gly-Leu- |
| | | Hap1-Leu-Ile- |
| | | Hap1-Ala-Ser- |
| | | Hap1-Ala-Ala- |
| | | Hap1-Asn-Ile- |
| | | Hap1-Thr-Gly- |
| | | Hap1-Leu-Glu- |
| | | Hap1-Gln-Thr- |
| | | Hap1-Thr-Tyr- |
| | | Hap1-Ser-Ala- |
| | | Hap1-Tyr-Ala- |
| | | Hap1-Ser-Pro- |
| | | Hap1-Leu-Val- |
| | | Leu-Val-Lys- |
| | | Hap2 |
| | | Hap1-Ser-Ser- |
| | | Hap1-Asp-Gly- |
| | | Hap1-Phe-Ser- |
| | | Hap1-Phe-Leu- |
| | | Phe-Leu-Lys- |
| | | Hap2 |
| | | Ser-Ser-Lys-Hap2 |
| | | Hap1-Ser-Phe- |
| | | Hap1-Thr-Leu- |
| | | Leu-Asp-Lys- |
| | | Hap2 |
| | | Hap1-Glu-Ile- |
| | | Hap1-Asn-Gly- |
| | | Hap1-Val-His- |
| | | Hap1-Gln-Ile- |
| | | Ile-Ser-Lys-Hap2 |
| | | Hap1-Ala- Leu- |
| | | Hap1-Leu-Ala- |
| | | Hap1-Val-Ala- |
| | | Hap1-Tyr-Phe- |
| | | Hap1-Leu-Ser- |
| | | Hap1-Ile-Ile- |
| | | Hap1-Glu-Thr- |
| | | Hap1-Asn-Val- |
| | | Hap1-Val-Thr- |
| | | Hap1-Leu-Cys- |
| | | Hap1-Glu-Phe- |
| | | Hap1-Ala-Met- |
| | | Hap1-Asp-Thr- |
| | | Hap1-Ile-Gln- |
| | | Hap1-Gln-Pro- |
| | | Hap1-Asp-Ser- |
| | | Hap1-Phe-Tyr- |
| | | Hap1-Phe-Pro- |
| | | Hap1-Leu-Phe- |
| | | Hap1-Thr-Val- |
| | | Hap1-Ser-Val- |
| | | Hap1-Glu-Arg |
| | | Hap1-Gly-Thr- |
| | | Hap1-Ser-Asn- |
| | | Hap1-Thr-Pro- |
| | | Hap1-Thr-Gln- |
| | | Hap1-Asn-Pro- |
| | | Ile-Leu-Lys-Hap2 |
| | | Hap1-Thr-Phe- |
| | | Pro- |
| | | Ile-Val-Lys-Hap2 |
| 49 | 77 epitopes* | Hap1-Val-Val- |
| | 2-3 amino acids plus the | Hap1-Asp-Leu- |
| | tag. | Hap1-Ala-Ile- |
| | All epitopes of solution | Cys-Lys-Hap2 |
| | 20, above, | Hap1-Ala-Val- |
| | are removed as | Hap1-Phe-Phe- |
| | candidate epitopes | Hap1-Ile-Phe- |
| | | Leu-Leu-Lys- |
| | | Hap2 |
| | | Hap1-Gly-Ala- |
| | | Hap1-Leu-Gln- |
| | | Hap1-Leu-Arg- |
| | | Hap1-Ser-Thr- |
| | | Hap1-Val-Asn- |
| | | Hap1-Asn-Ser- |
| | | Hap1-Gly-Leu- |
| | | Hap1-Ala-Ser- |
| | | Hap1-Asn-Ile- |
| | | Hap1-Tyr-Ala- |
| | | Hap1-Ser-Pro- |
| | | Hap1-Ser-Ser- |
| | | Hap1-Asp-Gly- |
| | | Hap1-Phe-Leu- |
| | | Hap1-Thr-Leu- |
| | | Hap1-Glu-Ile- |
| | | Hap1-Gln-Ile- |
| | | Ile-Ser-Lys-Hap2 |
| | | Hap1-Leu-Ala- |
| | | Hap1-Tyr-Phe- |
| | | Hap1-Leu-Ser- |
| | | Hap1-Ile-Ile- |
| | | Hap1-Glu-Thr- |
| | | Hap1-Asp-Thr- |
| | | Hap1-Leu-Phe- |
| | | Hap1-Thr-Val- |
| | | Hap1-Ser-Val- |
| | | Hap1-Ser-Gly- |
| | | Hap1-Ser-Ala- |
| | | Hap1-Ile-Gln- |
| | | Hap1-Leu-Cys- |
| | | Hap1-Phe-Ser- |
| | | Hap1-Leu-Glu- |
| | | Hap1-Thr-Gly- |
| | | Hap1-Phe-Tyr- |
| | | Hap1-Gly-Thr- |
| | | Hap1-Ala-Met- |
| | | Hap1-Gln-Gln- |
| | | Hap1-Thr-Gln- |
| | | Leu-Asp-Lys- |
| | | Hap2 |
| | | Hap1-Glu-Arg- |
| | | Phe-Leu-Lys- |
| | | Hap2 |
| | | Hap1-Thr-Thr- |
| | | Hap1-Ala-Leu- |
| | | Hap1-Ser-Phe- |
| | | Hap1-Leu-Ile- |
| | | Hap1-Val-His- |
| | | Hap1-Thr-Tyr- |
| | | Hap1-Asp-Ser- |
| | | Hap1-Ala-Gly- |
| | | His-Lys-Hap2 |
| | | Hap1-Asn-Gly- |
| | | Hap1-Val-Ala- |
| | | Hap1-Ser-Asn- |
| | | Leu-Val-Lys- |
| | | Hap2 |
| | | Ser-Ser-Lys-Hap2 |
| | | Hap1-Asn-Val- |
| | | Cys-Leu-Lys- |
| | | Hap2 |
| | | Hap1-Thr-Ser- |
| | | Hap1-Leu-Val- |
| | | Hap1-Gln-Thr- |
| | | Hap1-Ala-Ala- |
| | | Hap1-Thr-Pro- |
| | | Hap1-Val-Thr- |
| | | Hap1-Glu-Phe- |
| | | Hap1-Phe-Pro- |
| | | Hap1-Asn-Pro- |
| | | Hap1-Ala-Ala-Ile- |
| | | Ile-Val-Lys-Hap2 |
| 50 | 77 epitopes* | Hap1-Val-Val- |
| | 2-3 amino acids plus the | Hap1-Asp-Leu- |
| | tag. | Hap1-Ala-Ile- |
| | All epitopes of solution | Cys-Lys-Hap2 |
| | 20, above, | Hap1-Ala-Val- |
| | are removed as | Hap1-Phe-Phe- |
| | candidate epitopes | Hap1-Ile-Phe- |
| | | Leu-Leu-Lys- |
| | | Hap2 |
| | | Hap1-Gly-Ala- |
| | | Hap1-Leu-Gln- |
| | | Hap1-Leu-Arg- |
| | | Hap1-Ser-Thr- |
| | | Hap1-Val-Asn- |
| | | Hap1-Asn-Ser- |
| | | Hap1-Gly-Leu- |
| | | Hap1-Ala-Ser |
| | | Hap1-Asn-Ile- |
| | | Hap1-Tyr-Ala- |
| | | Hap1-Ser-Ser- |
| | | Hap1-Asp-Gly- |
| | | Hap1-Phe-Leu- |
| | | Hap1-Thr-Leu- |
| | | Hap1-Glu-Ile- |
| | | Hap1-Gln-Ile- |
| | | Ile-Ser-Lys-Hap2 |
| | | Hap1-Leu-Ala- |
| | | Hap1-Tyr-Phe- |
| | | Hap1-Leu-Ser- |
| | | Hap1-Ile-Ile- |
| | | Hap1-Leu-Phe- |
| | | Hap1-Thr-Val- |
| | | Hap1-Ser-Val- |
| | | Hap1-Ser-Gly- |
| | | Hap1-Ser-Ala- |
| | | Hap1-Leu-Cys- |
| | | Hap1-Phe-Ser- |
| | | Hap1-Leu-Glu- |
| | | Hap1-Thr-Gly- |
| | | Hap1-Phe-Tyr- |
| | | Hap1-Gly-Thr- |
| | | Hap1-Ala-Met- |
| | | Hap1-Gln-Gln- |
| | | Hap1-Thr-Gln- |
| | | Phe-Leu-Lys |
| | | Hap2 |
| | | Hap1-Thr-Thr- |
| | | Hap1-Ala-Leu- |
| | | Hap1-Ser-Phe- |
| | | Hap1-Leu-Ile- |
| | | Hap1-Thr-Tyr- |
| | | Hap1-Asp-Ser- |
| | | Hap1-Ala-Gly- |
| | | His-Lys-Hap2 |
| | | Hap1-Asn-Gly- |
| | | Hap1-Val-Ala- |
| | | Hap1-Ser-Asn- |
| | | Leu-Val-Lys- |
| | | Hap2 |
| | | Ser-Ser-Lys-Hap2 |
| | | Hap1-Asn-Val- |
| | | Hap1-Thr-Ser- |
| | | Hap1-Leu-Val- |
| | | Hap1-Gln-Thr- |
| | | Hap1-Ala-Ala- |
| | | Hap1-Glu-Phe- |
| | | Hap1-Val-His- |
| | | Hap1-Glu-Arg- |
| | | Hap1-Tyr-Asn- |
| | | Hap1-Ile-Gln- |
| | | Hap1-Ser-Pro- |
| | | Hap1-Glu-Thr- |
| | | Hap1-Phe-Pro- |
| | | Hap1-Val-Thr- |
| | | Hap1-Asp-Thr- |
| | | Cys-Leu-Lys- |
| | | Hap2 |
| | | Hap1-Thr-Pro- |
| | | Hap1-Thr-Arg- |
| | | Hap1-Asn-Pro- |
| | | Ile-Val-Lys-Hap2 |
| 51 | 77 epitopes* | Cys-Lys-Hap2 |
| | 2-3 amino acids plus the | Hap1-Ile-Phe- |
| | tag. | Leu-Leu-Lys- |
| | All epitopes of solution | Hap2 |
| | 20, above, | Hap1-Gly-Leu- |
| | are removed as | Hap1-Glu-Ile- |
| | candidate epitopes | Ile-Ser-Lys-Hap2 |
| | | Hap1-Leu-Ser- |
| | | Hap1-Ser-Gly- |
| | | Hap1-Ser-Ala- |
| | | Hap1-Thr-Ser- |
| | | Hap1-Glu-Thr- |
| | | Hap1-Leu-Phe- |
| | | Hap1-Val-Val- |
| | | Hap1-Val-Asn- |
| | | His-Lys-Hap2 |
| | | Hap1-Leu-Gln- |
| | | Leu-Asp-Lys- |
| | | Hap2 |
| | | Hap1-Ala-Ala- |
| | | Hap1-Asp-Thr- |
| | | Hap1-Phe-Ser- |
| | | Hap1-Ser-Thr- |
| | | Hap1-Leu-Val- |
| | | Hap1-Val-His- |
| | | Hap1-Thr-Thr- |
| | | Hap1-Asn-Val- |
| | | Hap1-Asn-Ser- |
| | | Hap1-Thr-Gln- |
| | | Hap1-Ala-Ser- |
| | | Phe-Leu-Lys- |
| | | Hap2 |
| | | Hap1-Leu-Glu- |
| | | Hap1-Leu-Cys- |
| | | Hap1-Leu-Ile- |
| | | Hap1-Ser-Pro- |
| | | Hap1-Gln-Ile- |
| | | Hap1-Phe-Leu- |
| | | Hap1-Thr-Val- |
| | | Hap1-Gln-Gln- |
| | | Hap1-Gly-Thr- |
| | | Hap1-Ala-Gly- |
| | | Hap1-Asp-Leu- |
| | | Hap1-Val-Ile- |
| | | Hap1-Ala-Met- |
| | | Hap1-Ser-Ser- |
| | | Hap1-Leu-Ala- |
| | | Hap1-Asp-Gly- |
| | | Ser-Ser-Lys-Hap2 |
| | | Hap1-Phe-Phe- |
| | | Hap1-Ile-Gln- |
| | | Hap1-Leu-Arg- |
| | | Hap1-Ser-Phe- |
| | | Hap1-Thr-Leu- |
| | | Hap1-Ala-Ile- |
| | | Hap1-Ser-Val- |
| | | Hap1-Ala-Val- |
| | | Hap1-Tyr-Phe- |
| | | Hap1-Thr-Tyr- |
| | | Cys-Leu-Lys- |
| | | Hap2 |
| | | Hap1-Gly-Ala- |
| | | Hap1-Gln-Thr- |
| | | Hap1-Asn-Ile- |
| | | Hap1-Glu-Arg- |
| | | Leu-Val-Lys- |
| | | Hap2 |
| | | Hap1-Ser-Asn- |
| | | Hap1-Tyr-Ala- |
| | | Hap1-Glu-Phe- |
| | | Hap1-Asp-Ser- |
| | | Hap1-Ile-Ile- |
| | | Hap1-Val-Ala- |
| | | Hap1-Thr-Pro- |
| | | Hap1-Asn-Gly- |
| | | Hap1-Thr-Gly- |
| | | Hap1-Ala-Leu- |
| | | Hap1-Phe-Tyr- |
| | | Hap1-Val-Thr- |
| | | Hap1-Ile-Leu- |
| | | Glu- |
| | | Val-Ser-Lys-Hap2 |
| | | Ile-Val-Lys-Hap2 |
| 52 | 77 epitopes*2-3 amino | His-Lys-Hap2 |
| | acids plus the tag.All | Hap1-Leu-Ser- |
| | epitopes of solution 20, | Cys-Lys-Hap2 |
| | above, are removed as | Hap1-Ser-Ser- |
| | candidate epitopes | Hap1-Leu-Ile- |
| | | Hap1-Ile-Val- |
| | | Hap1-Phe-Leu- |
| | | Hap1-Asp-Leu- |
| | | Hap1-Ser-Ala- |
| | | Hap1-Leu-Ala- |
| | | Hap1-Val-Ala- |
| | | Hap1-Thr-Thr- |
| | | Hap1-Leu-Gln- |
| | | Hap1-Asn-Val- |
| | | Hap1-Asn-Ile- |
| | | Hap1-Ala-Ala- |
| | | Hap1-Thr-Val- |
| | | Hap1-Asn-Ser- |
| | | Hap1-Ile-Gln- |
| | | Hap1-Asp-Thr- |
| | | Hap1-Glu-Arg- |
| | | Ile-Val-Lys-Hap2 |
| | | Hap1-Thr-Leu- |
| | | Hap1-Ile-Ala- |
| | | Hap1-Ile-Ile- |
| | | Hap1-Gln-Leu- |
| | | Hap1-Val-Asn- |
| | | Hap1-Asp-Phe- |
| | | Hap1-Ser-Asn- |
| | | Hap1-Gln-Ile- |
| | | Hap1-Asn-Phe- |
| | | Hap1-Asp-Ala- |
| | | Hap1-Val-Phe- |
| | | Leu-Leu-Lys- |
| | | Hap2 |
| | | Hap1-Ala-Ser- |
| | | Hap1-Val-Ile- |
| | | Leu-Val-Lys- |
| | | Hap2 |
| | | Hap1-Thr-Ser- |
| | | Hap1-Ser-Phe- |
| | | Ser-Ser-Lys-Hap2 |
| | | Hap1-Glu-Val- |
| | | Hap1-Gly-Leu- |
| | | Hap1-Tyr-Ser- |
| | | Leu-Phe-Lys- |
| | | Hap2 |
| | | Hap1-Glu-Thr- |
| | | Hap1-Ser-Thr- |
| | | Hap1-Ser-Asp- |
| | | Hap1-Gln-Pro- |
| | | Hap1-Leu-Val- |
| | | Hap1-Gln-Gln- |
| | | Hap1-Phe-Ser- |
| | | Hap1-Phe-Pro- |
| | | Hap1-Ile-Phe- |
| | | Hap1-Gln-Ala- |
| | | Hap1-Tyr-Leu- |
| | | Hap1-Leu-Arg- |
| | | Hap1-Val-Val- |
| | | Leu-Asp-Lys- |
| | | Hap2 |
| | | Hap1-Asp-Gly- |
| | | Hap1-Gln-Met- |
| | | Gly-Ser-Lys-Hap2 |
| | | Hap1-Leu-Phe- |
| | | Hap1-Thr-Gln- |
| | | Val-Thr-Lys-Hap2 |
| | | Ala-Tyr-Lys-Hap2 |
| | | Asn-Ser-Lys- |
| | | Hap2 |
| | | Hap1-Thr-Gly- |
| | | Hap1-Phe-Ile- |
| | | Hap1-Gly-Arg- |
| | | Leu-Ser-Lys-Hap2 |
| | | Hap1-Gln-Thr- |
| | | Hap1-Ala-Leu- |
| | | Hap1-Ile-Thr- |
| | | Ala-Ile-Lys-Hap2 |
| | | Hap1-Leu-Met- |
| | | Gly-Gln-Lys- |
| | | Hap2 |
| | | Hap1-Tyr-Ala- |
| 53 | 77 epitopes* | His-Lys-Hap2 |
| | 2-3 amino acids plus the | Hap1-Leu-Ser- |
| | tag. | Cys-Lys-Hap2 |
| | All epitopes of solution | Hap1-Ser-Ser- |
| | 20, above, | Hap1-Leu-Ile- |
| | are removed as | Hap1-Ile-Val- |
| | candidate epitopes | Hap1-Asp-Leu- |
| | | Hap1-Len-Ala- |
| | | Hap1-Thr-Thr- |
| | | Hap1-Asn-Ile- |
| | | Hap1-Thr-Val |
| | | Hap1-Asn-Ser- |
| | | Hap1-Ile-Gln |
| | | Hap1-Thr-Leu- |
| | | Hap1-Ile-Ala- |
| | | Hap1-Ile-Ile- |
| | | Hap1-Gln-Leu- |
| | | Hap1-Val-Asn- |
| | | Hap1-Ser-Asn- |
| | | Hap1-Asn-Phe- |
| | | Hap1-Asp-Ala- |
| | | Hap1-Val-Phe- |
| | | Leu-Leu-Lys- |
| | | Hap2 |
| | | Leu-Val-Lys- |
| | | Hap2 |
| | | Hap1-Thr-Ser- |
| | | Ser-Ser-Lys-Hap2 |
| | | Hap1-Gly-Leu- |
| | | Hap1-Tyr-Ser- |
| | | Hap1-Glu-Thr- |
| | | Hap1-Ser-Thr- |
| | | Hap1-Ser-Asp- |
| | | Hap1-Gln-Pro- |
| | | Hap1-Leu-Val- |
| | | Hap1-Phe-Ser- |
| | | Hap1-Ile-Phe- |
| | | Hap1-Tyr-Leu- |
| | | Hap1-Leu-Arg- |
| | | Leu-Asp-Lys- |
| | | Hap2 |
| | | Hap1-Asp-Gly- |
| | | Hap1-Gln-Met- |
| | | Gly-Ser-Lys-Hap2 |
| | | Hap1-Leu-Phe- |
| | | Val-Thr-Lys-Hap2 |
| | | Asn-Ser-Lys- |
| | | Hap2 |
| | | Hap1-Thr-Gly- |
| | | Hap1-Phe-Ile- |
| | | Hap1-Gly-Arg- |
| | | Leu-Ser-Lys-Hap2 |
| | | Hap1-Gln-Thr- |
| | | Hap1-Ala-Leu- |
| | | Hap1-Ala-Ser- |
| | | Hap1-Asp-Thr- |
| | | Hap1-Gln-Ile- |
| | | Hap1-Glu-Val- |
| | | Hap1-Leu-Met- |
| | | Hap1-Asn-Val- |
| | | Hap1-Glu-Arg- |
| | | Hap1-Val-Ala- |
| | | Hap1-Thr-Gln- |
| | | Hap1-Phe-Leu- |
| | | Leu-Phe-Lys- |
| | | Hap2 |
| | | Hap1-Ala-Ala- |
| | | Hap1-Ile-Thr- |
| | | Ala-Ile-Lys-Hap2 |
| | | Ala-Tyr-Lys-Hap2 |
| | | Hap1-Leu-Gln- |
| | | Hap1-Val-Val- |
| | | Hap1-Ser-Phe- |
| | | Hap1-Ser-Ala- |
| | | Hap1-Gln-Gln- |
| | | Hap1-Phe-Pro- |
| | | Hap1-Val-Ile- |
| | | Hap1-Asp-Phe- |
| | | Hap1-Gln-Ala- |
| | | Ile-Val-Lys-Hap2- |
| | | Gly-Gln-Lys- |
| | | Hap2 |
| | | Val-Phe-Lys- |
| | | Hap2 |
| 54 | 77 epitopes* | Hap1-Thr-Leu- |
| | 2-3 amino acids plus the | Hap1-Leu-Phe- |
| | tag. | Hap1-Ala-Leu- |
| | All epitopes of solution | Leu-Leu-Lys- |
| | 20, above, | Hap2 |
| | are removed as | Hap1-Leu-Ser- |
| | candidate epitopes | Hap1-Val-Phe- |
| | | Hap1-Ser-Asp- |
| | | Hap1-Leu-Ile- |
| | | Hap1-Gly-Leu- |
| | | Hap1-Asp-Leu- |
| | | Hap1-Ile-Phe- |
| | | Cys-Lys-Hap2 |
| | | Hap1-Leu-Ala- |
| | | Hap1-Thr-Arg- |
| | | Hap1-Cys-Val- |
| | | Hap1-Gln-Ala- |
| | | Hap1-Asp-Glu- |
| | | Hap1-Ser-Thr- |
| | | Hap1-Phe-Leu- |
| | | Hap1-Leu-Arg- |
| | | Hap1-Ala-Val- |
| | | Hap1-Glu-Ile- |
| | | Hap1-Glu-Phe- |
| | | Hap1-Leu-Val- |
| | | Hap1-Glu-Arg- |
| | | Hap1-Tyr-Leu- |
| | | Hap1-Leu-Gln- |
| | | Hap1-Asn-Gly- |
| | | Hap1-Ser-Ser- |
| | | Hap1-Val-Asp- |
| | | Hap1-Gly-Arg- |
| | | Leu-Phe-Lys- |
| | | Hap2 |
| | | Ser-Ser-Lys-Hap2 |
| | | Hap1-Ser-Ala- |
| | | Hap1-Asn-Ser- |
| | | Hap1-Asp-Ile- |
| | | Hap1-Thr-Ser- |
| | | Hap1-Trp-Leu- |
| | | Hap1-Asn-Ile- |
| | | Hap1-Phe-Tyr- |
| | | Hap1-Leu-Glu- |
| | | Hap1-Ser-Phe- |
| | | Hap1-Ile-Gln- |
| | | Val-Ser-Lys-Hap2 |
| | | Hap1-Val-Ile |
| | | Hap1-Asn-Phe |
| | | Ala-Ser-Lys-Hap2 |
| | | Hap1-Val-Val- |
| | | Hap1-Ile-Pro- |
| | | Hap1-Glu-Thr |
| | | Hap1-Asp-Gly- |
| | | Leu-Ser-Lys-Hap2 |
| | | Hap1-Thr-Thr- |
| | | Hap1-Ser-Gly- |
| | | Hap1-Phe-Glu- |
| | | Hap1-Ser-Val- |
| | | Hap1-Asn-Val- |
| | | Hap1-Thr-Val- |
| | | Hap1-Ala-Ser- |
| | | His-Lys-Hap2 |
| | | Ser-Leu-Lys-Hap2 |
| | | Glu-Asn-Lys- |
| | | Hap2 |
| | | Hap1-Gln-Met- |
| | | Hap1-Val-Asn- |
| | | Hap1-Tyr-Asn- |
| | | Leu-Val-Lys- |
| | | Hap2 |
| | | Hap1-Val-Ala- |
| | | Hap1-Gln-Leu- |
| | | Hap1-Ile-Ile- |
| | | Hap1-Gln-Thr- |
| | | Hap1-Gln-Ile- |
| | | Hap1-Phe-Pro- |
| | | Asp-Asp-Lys- |
| | | Hap2 |
| | | Gly-Gln-Lys- |
| | | Hap2 |
| | | Hap1-Trp-Ala- |
| | | Hap1-Asp-Ser- |
| | | Ile-Val-Lys-Hap2 |
| 55 | 77 epitopes* | Cys-Lys-Hap2 |
| | 2-3 amino acids plus the | Hap1-Leu-Phe- |
| | tag. | Hap1-Ala-Ser- |
| | All epitopes of solution | Hap1-Ser-Thr- |
| | 20, above, | Phe-Leu-Lys- |
| | are removed as | Hap2 |
| | candidate epitopes | Hap1-Ser-Ser- |
| | | Hap1-Leu-Ser- |
| | | Hap1-Asp-Leu- |
| | | Hap1-Asn-Ser- |
| | | Hap1-Leu-Glu- |
| | | Hap1-Thr-Leu- |
| | | Hap1-Ile-Thr- |
| | | Hap1-Leu-Gln- |
| | | Hap1-Val-Thr- |
| | | Leu-Leu-Lys- |
| | | Hap2 |
| | | Hap1-Thr-Ser- |
| | | Hap1-Leu-Arg- |
| | | Hap1-Glu-Thr- |
| | | Hap1-Ser-Ala- |
| | | Hap1-Gln-Ile- |
| | | Hap1-Leu-Cys- |
| | | Hap1-Leu-Ala- |
| | | Ser-Ser-Lys-Hap2 |
| | | Hap1-Thr-Gly- |
| | | Hap1-Gln-Leu- |
| | | Hap1-Gln-Thr- |
| | | Hap1-Ala-Leu- |
| | | Leu-Ser-Lys-Hap2 |
| | | Hap1-Asn-Ile- |
| | | Hap1-Ile-Ile- |
| | | Hap1-Ala-Ile- |
| | | Hap1-Tyr-Ala- |
| | | Leu-Thr-Lys- |
| | | Hap2 |
| | | Hap1-Thr-Val- |
| | | Hap1-Ser-Phe- |
| | | Ile-Ser-Lys-Hap2 |
| | | Hap1-Ala-Ala- |
| | | Hap1-Val-Val- |
| | | Hap1-Gly-Ala- |
| | | Leu-Val-Lys- |
| | | Hap2 |
| | | Hap1-Gln-Ser- |
| | | Hap1-Thr-Thr- |
| | | Hap1-Phe-Phe- |
| | | Ser-Thr-Lys-Hap2 |
| | | Hap1-Thr-Arg- |
| | | Hap1-Gly-Leu- |
| | | Hap1-Ser-Gln- |
| | | Hap1-Glu-Arg- |
| | | Hap1-Phe-Ser- |
| | | Ile-Val-Lys-Hap2 |
| | | Hap1-Phe-Leu- |
| | | Hap1-Val-Ile- |
| | | Hap1-Ala-Val- |
| | | Hap1-Val-Ala- |
| | | Hap1-Val-Asn- |
| | | Hap1-Ser-Asp- |
| | | Hap1-Gln-Pro- |
| | | His-Lys-Hap2 |
| | | Leu-Asp-Lys- |
| | | Hap2 |
| | | Hap1-Glu-Phe- |
| | | Phe-Ser-Lys-Hap2 |
| | | Hapl-Tyr-Thr- |
| | | Val-Leu-Lys- |
| | | Hap2 |
| | | Gln-Gly-Lys- |
| | | Hap2 |
| | | Gly-Gln-Lys- |
| | | Hap2 |
| | | Hap1-Ser-Val- |
| | | Hap1-Ile-Arg- |
| | | Hap1-Leu-Val- |
| | | Val-Ser-Lys-Hap2 |
| | | Hap1-Glu-Val- |
| | | Hap1-Asp-Thr- |
| | | Hap1-Asp-Gly- |
| | | Ile-Ile-Lys-Hap2 |
| | | Hap1-Asn-Val- |
| | | Hap1-Thr-Tyr- |
| | | Hap1-Phe-Pro- |
| | | Hap1-Gln-Met- |
| 56 | Pruned E. coli/Trypsin | Leu-Lys-Hap2 |
| | model. | Asp-Lys-Hap2 |
| | 32 epitopes* | Gly-Lys-Hap2 |
| | 2-3 amino acids plus the | Met-Lys-Hap2 |
| | tag. | Val-Lys-Hap2 |
| | No epitope having all | Ile-Lys-Hap2 |
| | hydrophobic amino acid | Ser-Lys-Hap2 |
| | residues. | Hap1-Leu-Arg- |
| | | Gln-Lys-Hap2 |
| | | Thr-Lys-Hap2 |
| | | His-Lys-Hap2 |
| | | Hap1-Glu-Leu- |
| | | Phe-Lys-Hap2 |
| | | Asn-Lys-Hap2 |
| | | Pro-Lys-Hap2 |
| | | Hap1-Gln-Ala- |
| | | Ala-Lys-Hap2 |
| | | Hap1-Ala-Gly- |
| | | Trp-Lys-Hap2 |
| | | Tyr-Lys-Hap2 |
| | | Glu-Lys-Hap2 |
| | | Hap1-Thr-Leu- |
| | | Hap1-Ile-Gly- |
| | | Hap1-Ser-Arg- |
| | | Hap1-Val-Arg- |
| | | Hap1-Glu-Thr- |
| | | Hap1-Ala-Glu- |
| | | Hap1-Ile-Gln- |
| | | Hap1-Asn-Gly- |
| | | Glu-Leu-Lys- |
| | | Hap2 |
| | | Hap1-Glu-Ala- |
| | | Hap1-Ser-Leu- |

*one protein pair (out of approximately (6000 proteins)2/2 = 18 million protein pairs) was not resolvable by these solutions.

What is claimed is:

1. A method for identifying a protein, the method comprising:
cleaving the protein with a proteolytic agent to produce peptide fragments;
providing an array comprising a solution set of binding reagents;
contacting the peptide fragments with the array to promote specific interactions between the fragments and the array;
detecting the binding pattern of the peptide fragments on the array; and
comparing the binding pattern of the peptide fragments to a reference set.

2. The method of claim 1, wherein the reference set is contained in a database.

3. The method of claim 1, wherein the proteolytic agent is selected from the group consisting of Arg-C proteinase, Asp-N endopeptidase, BNPS-skatole, caspase 1, caspase 2, caspase 3, caspase 4, caspase 5, caspase 6, caspase 7, caspase 8, caspase 9, caspase 10, chymotrypsin, clostripain (clostridiopeptidase B), CNBr, factor Xa, formic acid, glutamyl endopeptidase, granzyme B, hydroxylamine (NH2OH), iodosobenzoic acid, lys-C proteinase, NTCB+Ni (2-nitro-5-thiocyanobenzoic acid), pepsin, proline-endopeptidase, proteinase K, staphylococcal peptidase I, thermolysin, thrombin, and trypsin.

4. The method of claim 1, wherein the proteolytic agent is trypsin.

5. The method of claim 1, wherein the peptide fragments are labeled with a tag.

6. The method of claim 5, wherein either the N-terminus or the C-terminus of the peptide fragments are labeled.

7. The method of claim 5, wherein the tag is fluorescent.

8. The method of claim 1, wherein the binding reagent is selected from the group consisting of antibodys, single chain fragments (ScFv), F(ab) fragments, and aptamers.

9. The method of claim 8, wherein the binding reagent is an antibody.

10. The method of claim 9, wherein the antibody is a monoclonal antibody.

11. The method of claim 1, wherein the array comprises the binding reagents in a spatially-addressable form.

12. The method of claim 5, wherein the binding reagent interacts with the tag and at least one amino acid of the peptide fragment adjacent to the tag.

13. The method of claim 1, wherein the reference set is provided in computer readable form.

14. The method of claim 1, wherein the reference set is provided in printed form.

15. A method for forming a solution set of at least one epitope, the solution set to identify at least two proteins, the method comprising:
forming at least one protein group by associating each of the at least two proteins based on whether the proteins are undistinguished by the solution set, and,
updating the solution set with a maximum epitope that divides a maximum number of protein groups.

16. A method according to claim 15, wherein associating includes associating such that associated proteins are undistinguished by the solution set and unassociated proteins are distinguished by the solution set.

17. A method according to claim 15, wherein the solution set is initialized to the empty set.

18. A method according to claim 15, wherein the solution set is initialized to the empty set, and forming at least one association includes associating all of the at least two proteins based on the initialized solution set.

19. A method according to claim 15, further including iteratively performing the forming and the updating until each of the at least two proteins is unassociated with any of the other at least two proteins.

20. A method according to claim 15, wherein updating includes updating the at least one protein group based on whether the associated proteins are undistinguished by the updated solution set.

21. A method according to claim 15, wherein associating includes associating such that, for each associated protein in a selected one of the at least one protein group, and for a selected one of the at least one epitope in the solution set, the selected protein group proteins either include the selected epitope, or the selected protein group proteins do not include the selected epitope.

22. A method according to claim 15, wherein updating the solution set includes
determining that at least two epitopes divide a maximum number of the at least one protein group, and,
selecting one of the at least two epitopes that divides a maximum number of the at least one protein group.

23. A method according to claim 15, wherein updating the solution set includes,
determining that at least two epitopes divide a maximum number of the at least one protein group, and,
randomly selecting one of the at least two epitopes that divides a maximum number of the at least one protein group.

24. A method according to claim 15, wherein the at least two proteins include a protein catalog.

25. A method according to claim 15, wherein the at least one epitope is based on cleaving the at least two proteins with at least one proteolytic agent.

26. A method according to claim 15, wherein associating includes determining that at least one of the at least two proteins is unassociated with another of the at least two proteins.

27. A method according to claim 15, wherein associating includes assigning a label to the at least one protein group.

28. A method according to claim 15, wherein updating the solution set includes assigning to at least one of the at least one protein groups, a group score based on whether a selected epitope can distinguish associated proteins in the at least one protein group.

29. A method according to claim 15, wherein updating the solution set includes assigning to at least one of the at least one protein group, a group score based on whether a selected epitope is included in at least one, but not all of the protein group proteins.

30. A method according to claim 15, further including providing a database that associates the epitopes of the at least two proteins based on at least one proteolytic agent.

31. A method according to claim 30, wherein the database includes a label for associated proteins.

32. A method according to claim 15, wherein associated proteins are associated using at least one of at least one database, at least one linked list, at least one queue, at least one hash table, and at least one tree.

33. A method according to claim 15, including association means for associating the associated proteins.

35. A method according to claim 15, wherein updating includes determining the maximum epitope by computing a composite group score for at least one epitope that is not an element of the solution set.

36. A method according to claim 15, wherein updating includes,
computing at least one group score for at least one epitope, the at least one group score corresponding to at least one of the at least one protein group, and
generating a composite group for the at least one epitope score based on the at least one group score.

37. A method according to claim 36, wherein updating includes selecting as the maximum epitope, the at least one epitope with the maximum composite group score.

38. A method according to claim 15, wherein updating includes computing a group score based on the number of occurrences of an epitope in at least one selected of the at least one protein group, and a number of proteins in the selected protein group.

39. A method according to claim 15, further including associating the maximum epitope with a binding reagent.

40. A method according to claim 15, further including associating the maximum epitope with a binding reagent in a chip.

41. A method according to claim 15, further including forming a representation of the at least two proteins based on the maximum epitope.

42. A method according to claim 15, further including forming a representation of the at least two proteins based on the epitopes in the solution set.

43. A method according to claim 15, further including forming a binary representation of the at least two proteins based on whether the at least two proteins include the maximum epitope.

44. A method according to claim 15, wherein the maximum epitope is included in at least one of the at least two proteins.

45. A method according to claim 15, further including:
repeating forming a protein group and updating until the at least two proteins are unassociated with another of the at least two proteins, and,
forming a solution set based on the maximum epitopes.

46. A method according to claim 45, further including
eliminating at least some of the epitopes in the solution set, and,
based on the eliminated epitopes and the epitopes in the solution set, repeating forming at least one protein group and updating until the at least two proteins are unassociated with another of the at least two proteins.

47. A method for identifying a solution set of epitopes to identify at least two proteins, the method comprising:
determining the epitopes in the at least two proteins based on one or more proteolytic agents, and,
applying a randomized greedy algorithm to the determined epitopes to distinguish the solution set of epitopes.

48. A method according to claim 47, further including applying a local search algorithm to the solution set.

49. A method according to claim 47, further including iteratively applying a local search algorithm to the solution set.

50. A method according to claim 47, further including associating at least one of the epitopes in the solution set with a binding reagent.

51. A method according to claim 47, further including generating a binary representation for the at least two proteins based on the solution set.

52. A method according to claim 47, wherein applying a randomized greedy algorithm includes forming at least one protein group by associating the at least two proteins based on whether the at least two proteins are distinguished by the solution set.

53. A method according to claim 47, wherein applying a randomized greedy algorithm includes,
identifying a maximum epitope from the determined epitopes where the maximum epitope distinguishes at least as many pairs of the at least two proteins as at least one of the other determined epitopes,
associating the maximum epitope with a solution set,
removing the maximum epitope from the set of determined epitopes, and,
repeating the identifying, associating, and removing until every pair of the at least two proteins are distinguished by the epitopes associated with the solution set.

54. A method according to claim 47, wherein the at least two proteins are undistinguished by molecular mass.

55. A method for identifying at least one protein in a protein catalog, the method comprising:
determining epitopes in the protein catalog based on cleaving the protein catalog proteins with at least one proteolytic agent,
using a randomized greedy algorithm to identify a solution set of the determined epitopes that can distinguish the protein catalog proteins,
forming a chip based on binding reagents associated with the solution set of the determined epitopes,
obtaining a signature from the chip based on at least one protein in the protein catalog, and,
associating the signature with the at least one protein.

56. A method according to claim 55, further including identifying a signature for the at least one protein in the protein catalog, the signature based on the solution set of the determined epitopes.

57. A method according to claim 56, wherein associating the signature includes comparing the signature with the identified signature for the at least one protein in a protein catalog.

57. A method according to claim 55, further including using a local search algorithm with the greedy algorithm.

58. A method for generating an identifier for at least one protein in a protein catalog, the method comprising:
determining epitopes in the protein catalog based on cleaving the protein catalog proteins with at least one proteolytic agent,
identifying a solution set that includes a solution set of determined epitopes that distinguish the proteins, and,
associating an identifier with the at least one protein based on whether the at least one protein includes the epitopes in the solution set.

59. A method according to claim 58, wherein associating includes, for each epitope in the solution set, assigning a binary digit to the at least one protein based on whether at least one the protein includes the epitope.

60. A method according to claim 58, wherein identifying includes identifying based on a randomized greedy algorithm.

61. A method according to claim 60, further including a local search algorithm.

62. A method according to claim 58, wherein identifying includes:
associating protein catalog proteins based on whether the protein catalog proteins are undistinguished by the solution set,
updating the solution set with a maximum epitope that divides a maximum number of the associations, and,
repeating the forming and associating until the protein catalog proteins are unassociated with any other protein catalog protein.

63. A method according to claim 62, wherein associating an identifier is performed based on the number of repeats of the forming and the updating.

64. A method according to claim 58, wherein associating an identifier includes associating a binary number based on the solution set.

65. A method according to claim 58, further including associating the at least one protein with the identifier.

66. A processor-readable medium for storing data regarding a protein catalog, the medium comprising,
at least one protein name associated with at least one protein catalog protein, and,
for each of the at least one protein name, a protein identifier based on a solution set of epitopes for distinguishing the at least one protein catalog protein from other protein catalog proteins,
wherein the at least one protein name and the protein identifier are associated.

67. A processor-readable medium according to claim 66, wherein the at least one protein name is alphanumeric.

68. A processor-readable medium according to claim 66, wherein the protein identifier is binary.

69. A processor-readable medium according to claim 66, wherein the protein identifier is alphanumeric.

70. A processor-readable medium according to claim 66, further including, for the at least one protein name, an association with at least one epitope included in the at least one protein catalog protein associated with the at least one protein name.

71. A processor-readable medium according to claim 66, further including an association between the at least one protein name and a protein signature, wherein the protein signature is based upon a chip that includes binding reagents, wherein the binding reagents correspond to the solution set of epitopes.

72. A processor-readable medium according to claim 66, wherein the at least one protein name and the protein identifier are associated by at least one of at least one database, at least one queue, at least one linked list, at least one hash table, and at least one tree.

73. A chip for identifying at least one protein in a protein catalog, the chip comprising binding reagents that are associated with a solution set of epitopes, wherein the solution set of epitopes are determined by a method that includes:
determining epitopes in the protein catalog based on cleaving the protein catalog proteins with at least one proteolytic agent,
initializing the solution set of epitopes to be the empty set,
associating protein catalog proteins based on whether the protein catalog proteins are undistinguished by the solution set,
updating the solution set with a maximum epitope that divides a maximum number of the associations, and,
repeating the associating and updating until the protein catalog proteins are unassociated with any other protein catalog protein.

74. A method for evaluating a set of epitopes for identifying a protein in a protein catalog, the method comprising,
providing a chip including binding reagents associated with the set of epitopes,
selecting at least two proteins from the protein catalog,
determining a signature of the at least two proteins based on the chip,
adding errors to the signature to form an augmented signature, and,
computing a significance score for unidentified protein in the protein catalog, the significance score based on binding sites in unidentified protein catalog proteins and the augmented signature,
identifying a protein from the unidentified protein catalog proteins based on the largest significance score,
determining a signature of the identified protein,
removing the signature of the identified protein from the augmented signature,
repeating computing a significance score for each unidentified protein and identifying, until a number of proteins equal to the at least two selected proteins are identified, and,
comparing the identified proteins to the at least two selected proteins.

75. A method according to claim 74, further including updating a counter when the identified proteins are equivalent to the at least two selected proteins.

76. A method according to claim 74, further including returning to selecting at least two proteins and continuing to comparing the identified proteins.

77. A method according to claim 74, wherein the significance score for a protein is based on the number of binding sites in the protein catalog for a selected epitope as compared to the total number of binding sites in the protein catalog.

78. A method according to claim 74, wherein adding errors includes adding at least one of false negative and false positives.

79. A method according to claim 74, wherein adding errors includes adding errors based on at least one probability distribution.

80. A computer product disposed on a computer readable medium, the computer product for forming a solution set of at least one epitope, the solution set to identify at least two proteins, the computer product including instructions for causing a processor to:
form at least one protein group by associating each of the at least two proteins based on whether the proteins are undistinguished by the solution set, and,
update the solution set with a maximum epitope that divides a maximum number of the protein groups.

81. A computer product according to claim 80, wherein the instructions to associate include instructions to form at least one protein group.

82. A computer product according to claim 80, wherein the instructions to associate include instructions to associate such that associated proteins are undistinguished by the solution set and unassociated proteins are distinguished by the solution set.

83. A computer product according to claim 80, further including instructions to iteratively perform the instructions to form and update until each of the at least two proteins is unassociated with any of the other at least two proteins.

84. A computer product according to claim 80, wherein the instructions to update includes instructions to update the at least one protein group based on whether the associated proteins are undistinguished by the updated solution set.

85. A computer product according to claim 80, wherein the instructions to associate include instructions to associate such that, for each associated protein in a selected one of the at least one protein group, and for a selected one of the at least one epitope in the solution set, the selected protein group proteins either include the selected epitope, or the selected protein group proteins do not include the selected epitope.

86. A computer product according to claim 80, wherein the instructions to update the solution set include instructions to
determine that at least two epitopes divide a maximum number of the at least one protein group, and,
select one of the at least two epitopes that divides a maximum number of the at least one protein group.

87. A computer product according to claim 80, wherein the instructions to update the solution set include instructions to
determine that at least two epitopes divide a maximum number of the at least one protein group, and,
randomly select one of the at least two epitopes that divides a maximum number of the at least one protein group.

88. A computer product according to claim 80, wherein the at least two proteins include a protein catalog.

89. A computer product according to claim 80, wherein the at least one epitope is based on cleaving the at least two proteins with at least one proteolytic agent.

90. A computer product according to claim 80, wherein the instructions to associate include instructions to determine that at least one of the at least two proteins is not associated with another of the at least two proteins.

91. A computer product according to claim 80, wherein the instructions to associate includes instructions to assign a label to the at least one protein group.

92. A computer product according to claim 80, wherein the instructions to update the solution set include the instructions to assign to at least one of the at least one protein group, a group score based on whether a selected epitope is included in at least one, but not all of the protein group proteins.

93. A computer product according to claim 80, wherein the instructions to update the solution set include instructions to assign to at least one of the at least one protein groups, a group score based on whether a selected epitope can distinguish associated proteins in the at least one protein group.

94. A computer product according to claim 80, further including instructions to provide a database that associates the epitopes of the at least two proteins based on at least one proteolytic agent.

95. A computer product according to claim 94, wherein the database includes a label for associated proteins.

96. A computer product according to claim 80, wherein associated proteins are associated using at least one of at least one database, at least one linked list, at least one queue, at least one hash table, and at least one tree.

97. A computer product according to claim 80, including association means for associating the associated proteins.

98. A computer product according to claim 80, wherein the instructions to update include instructions to determine the maximum epitope by computing a composite group score for an epitope.

99. A computer product according to claim 80, wherein the instructions to update include instructions to
compute at least one group score for at least one epitope, the at least one group score corresponding to at least one of the at least one protein group, and
generate a composite group for the at least one epitope score based on the at least one group score.

100. A computer product according to claim 99, wherein the instructions to update include instructions to select as the maximum epitope, the at least one epitope with the maximum composite group score.

101. A computer product according to claim 80, wherein the instructions to update include instructions to compute a group score based on the number of occurrences of an epitope in at least one selected of the at least one protein group, and a number of proteins in the selected protein group.

102. A computer product according to claim 80, further including instructions to associate the maximum epitope with a binding reagent.

103. A computer product according to claim 80, further including instructions to associate the maximum epitope with a binding reagent in a chip.

104. A computer product according to claim 80, further including instructions to form a representation of the at least two proteins based on the maximum epitope.

105. A computer product according to claim 80, further including instructions to form a binary representation of the at least two proteins based on whether the at least two proteins include the maximum epitope.

106. A computer product according to claim 80, wherein the maximum epitope is included in at least one of the at least two proteins.

107. A computer product according to claim 80, further including instructions to:
repeat the instructions to form a protein group and update until the at least two proteins are unassociated with another of the at least two proteins, and,
instructions to form a solution set based on the maximum epitopes.

108. A method according to claim 107, further including instructions to:
eliminate at least some of the epitopes in the solution set, and,
based on the eliminated epitopes and the epitopes in the solution set, repeat the instructions to form at least one protein group and updating until the at least two proteins are unassociated with another of the at least two proteins.
